(19) United States
(12) Patent Application Publication
(10) Pub. No.: US 2008/0096193 A1
(43) Pub. Date: Apr. 24, 2008

(54) Methods and compositions for detecting polynucleotides

(76) Inventors: Charles Robert Bupp, Lafayete, CA (US); K. Yeon Choi, St. Paul, MN (US); Heather Koshinsky, El Cerrito, CA (US); Christopher Nulf, Berkeley, CA (US); Mickey Urdea, Alamo, CA (US); Miaomiao Wang, El Cerrito, CA (US); Michael Zwick, Vacaville, CA (US)

Correspondence Address: DONALD SILVERT;Brinks Hofer Gilson & Lione, Suite 3600, 455 N. Cityfron Plaza Drive, Chicago, IL, 60611, US

(21) Appl. No.: 11/362,359

(22) Filed: Oct. 24, 2006

(51) Int. Cl. C12Q 1/68; C07H 21/04; C07D 417/02

(52) U.S. Cl. 435/6; 536/25.34; 548/156

(57) ABSTRACT

Methods of determining the presence or amount of a target polynucleotide in a sample are provided. A sample that contains a target polynucleotide, a nucleic acid analog that is complementary to a target nucleic acid sequence of the target polynucleotide, and a dye for which the rate of change in an optical property is different in the presence and absence of a target polynucleotide/nucleic acid analog hybrid are combined to produce a reaction mixture. The rate of change in an optical property of the dye in the reaction mixture is compared to a reference value characteristic of the rate of change in the optical property of the dye in a similar reaction mixture containing a known amount of a polynucleotide/nucleic acid analog hybrid to determine a relative rate of change in the optical property. The relative rate of change in the optical property of dye in the reaction mixture is correlated with the presence or amount of the specified target polynucleotide in the sample.

### RELATED APPLICATIONS

This application claims priority from U.S. Provisional Application Ser. No. 60/655,929, filed Feb. 23, 2005, which is incorporated herein by reference in its entirety.

### FIELD

The present application relates to the field of diagnostics. More particularly, the invention disclosed herein is directed to methods, compositions, and kits for detecting pathogens, toxins, or other agents or factors that are desirably detected or measured.

### BACKGROUND

There is a great need to detect and quantify various molecular species, such as polynucleotides, polypeptides, carbohydrates, lipids, and small molecules. For example, current methods of detecting a polynucleotide, such as those associated with pathogens, pathogen infection, human genes associated with diseases and disorders, altered physiology or physiological conditions, genetically modified organisms (GMOs, i.e., organisms with transgenic DNA), biowarfare agents, veterinary applications, and agricultural applications presently rely on complex methods, such as the polymerase chain reaction (PCR), nucleic acid sequence-based amplification (NASBA), transcription-mediated amplification (TMA), or branched DNA PCR (bDNA-PCR). These methods require skilled personnel and specialized equipment. Further, the methods are generally incapable of determining the presence or quantity of polynucleotides in crude cell and tissue extracts. There are similar difficulties in the existing immunoassays for detecting antigens. For example, antigens associated with blood coagulation disorders (e.g., F 1+2; Dade Behring, Bannockburn, Ill.), hepatitis infection (e.g., hepatitis B surface antigen; Abbott Laboratories, Abbott Park, Ill.), cancer-detection (e.g., gastrointestinal stromal tumor-specific antigens; Ventana Medical Systems, Inc., Tucson, Ariz.); acute pancreatitis (e.g., pancreatic elastase; Schebo-Biotech AG, Giessen, Germany), prostate cancer (e.g., PSA; Beckman-Coulter, Inc., Fullerton, Calif.), and the like are all based on multi-step ELISA immunoassays that require skilled personnel and specialized equipment to run.

Accordingly, there is a great need for a convenient, fast and economical method of detection, identification, and quantification of various molecules, such as polynucleotides and antigens. Reducing the complexity and increasing the reliability of such tests are among the features that would be desireably improved.

### SUMMARY

Applicants have developed methods of determining the presence or amount of a target polynucleotide in a sample. A sample that is desirably tested for the presence or amount of a target polynucleotide is included in one of two alternative reaction mixtures. The choice of reaction mixture depends on whether the assay to be used involves the direct or indirect hybridization of a nucleic acid analog (“NAA”) and the target polynucleotide, i.e., is the nucleic acid analog sequence part of the query sequence and the reactive site or is the nucleic acid analog sequence not part of the query sequence. If direct hybridization is used, then the mixture includes the sample, a first nucleic acid analog (“NAA1”), and a dye. If indirect hybridization is used, then the mixture includes a secondary polynucleotide that has a portion that is complementary to a segment of the target polynucleotide and a segment that is complementary to the nucleic acid analog. One reaction mixture includes combined with a nucleic acid analog that is complementary to a target nucleic acid sequence of the target polynucleotide and a dye to produce a reaction mixture. The reaction mixture has an observable optical property. If the target nucleic acid sequence is present in the target polynucleotide, then a nucleic acid analog/target polynucleotide (“NAA/TP”) hybrid forms in the reaction mixture and affects the observable optical property thereof in a qualitative and/or quantitative manner. A qualitative change in the optical property, as one nonlimiting example, can be a change in color, as from blue to purple, for example. A quantitative change in the optical property, again as one nonlimiting example, can be a change in intensity (e.g., darker versus lighter) of substantially the same color. The rate of change in the optical property is preferably different in the presence and absence of the NAA/TP hybrid, which preferably correlates to the concentration or amount of the target polynucleotide in the sample. Accordingly, characteristic optical property changes allow one to determine the presence or amount of the target polynucleotide.

The dye that is included contributes the observable optical property of the mixture. Preferred dyes used in the context of the present invention are compounds represented by formula (I), or a salt or betaine thereof:

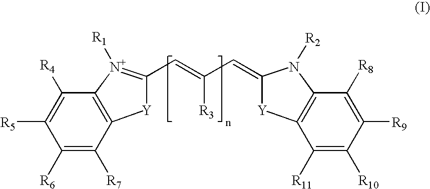

wherein, independently at each occurrence:
R₁ and R₂ are each independently selected from hydrogen, alkyl, alkenyl, alkynyl, heteralkyl, heteroalkenyl, heteroalkynyl, aryl, acyl, heteroacyl, heteroaryl, hydroxyl, alkoxy, carbonyl, sulfinyl, sulfonyl, and amino groups;
R₃ is selected from the group consisting of hydrogen, alkyl, alkenyl, alkynyl, aryl, heteroalkyl, heteroalkenyl, heteroalkynyl, aryl, acyl, heteroacyl, heteroaryl, aryl, alkyl, heteroarylakyl, hydroxyl, alkoxy, halo, carbonyl, sulfinyl, sulfonyl, and amino groups;
R₄, R₅, R₆, R₇, R₈, R₉, R₁₀, R₁₁, R₁₂, and R₁₃ are each independently selected from the group consisting of hydrogen, halogen, alkyl, alkenyl, alkynyl, heteroalkyl, heteroalkenyl, heteroalkynyl, aryl, acyl, heteroacyl, heteroaryl, aryl, alkyl, heteroarylakyl, hydroxyl, alkoxy, carbonyl, sulfinyl, and sulfonyl groups;
n is 0 1, 2, 3, 4, or 5; and
each Y is independently selected from the group consisting of —CR₁₂═CR₁₃—, sulfur, nitrogen, and oxygen. The preferred dye can be used as a suitable salt or betaine of any compound represented by formula (I). A more preferred dye is the compound of formula (I), wherein Y is sulfur or —CR₁₂═CR₁₃—. A yet more preferred dye is the compound of formula (I), wherein Y is sulfur.

The rate of change in the optical property of the mixture is preferably compared to a reference value that is characteristic of the rate of change in the optical property of a similar mixture containing a known amount of a NAA/TP hybrid to determine a relative rate of change in the optical property. The relative rate of change in the optical property of the mixture is correlated with the presence or amount of the target polynucleotide in the sample. More preferably, the rate of change in the optical property is usefully employed to determine the concentration or amount of the target polynucleotide.

A detergent is preferably also added to the mixture prior to comparing the rate of change therein of the optical property to a reference value. As used in this application, the term “detergent” is defined as any substance that reduces the surface tension of water, and is used synonymously with the term “surfactant”. In certain embodiments, the detergent can be a cationic detergent, anionic detergent, nonionic detergent, or a zwitterionic detergent. Preferably, the detergent is nonionic. When the target polynucleotide is contained in intact cells, the detergent preferably has a suitable concentration to permeabilize and/or lyse the cells.

The nucleic acid analog is preferably an achiral peptide nucleic acid (referred to herein as any of “non-chiral PNA”, “achiral PNA”, or “ncPNA”), a chiral peptide nucleic acid (referred to herein as “chiral PNA” or “cPNA”), a locked nucleic acid (“LNA”), a threose nucleic acid (“TNA”), a metal-linked nucleic acid, or a morpholino nucleic acid. More preferably, the nucleic acid analog is a cPNA or a ncPNA. Yet more preferably, the nucleic acid analog is a ncPNA. In certain embodiments, the length of the target polynucleotide is greater than about 400 bases. In certain other embodiments, the target is less than 400 bases. In certain embodiments, the nucleic acid analog is greater than about 4 nucleic acid bases in length and less than about 24 nucleic acid bases in length. Preferably, the nucleic acid analog is about 12 nucleic acid bases in length, however the method can be operated using a wide range of lengths of the nucleic acid analog, as is detailed herein.

In another variation of the above method, a sample, a nucleic acid analog that is complementary to at least a segment of the target polynucleotide, and a dye are combined to produce a mixture. The dye is the compound of formula (I), or a salt or betaine thereof. A stimulus is applied to the mixture, which stimulus is preferably a light stimulus. The mixture has an observable optical property, which changes in the presence or absence of a NAA/TP hybrid. In a preferred embodiment, the optical property that is observed in the context of the present invention is absorbance or fluorescence. In a more preferred embodiment, the observed optical property is absorbance, the intensity of which varies. Preferably, a decrease in the intensity of the absorbance of the mixture is correlated to the presence or amount of the specified target polynucleotide in the sample.

In another preferred method of determining the presence or amount of a target polynucleotide in a sample, the sample, a non-PNA nucleic acid analog that is complementary to at least a segment of the target polynucleotide, and a dye are combined to produce a mixture. The dye is the compound of formula (I), or a salt or betaine thereof. The mixture preferably has a different optical property in the presence and absence of a NAA/TP hybrid. A change in the optical property of the mixture is observed to determine the presence or quantity of target polynucleotide in the sample. In more preferred embodiments, the non-PNA nucleic acid analog is a locked nucleic acid (LNA), a threose nucleic acid (TNA), a metal-linked nucleic acid, or a morpholino nucleic acid.

The present application is further directed to a composition comprising a dye; preferably, the composition further comprises a surfactant. In one embodiment, the composition includes the dye, wherein the dye is a compound according to formula (I) or a salt or betaine thereof, as described above. More preferably, the composition includes the compound of formula (I), wherein Y is independently selected from —CR₁₂═CR₁₃—, sulfur, or oxygen. Yet more preferably, the composition includes the compound of formula (I), wherein Y is —CR₁₂═CR₁₃— or sulfur. Even more preferably, the composition includes the compound of Formula (I), wherein Y is sulfur. An alternative preferred composition includes the compound of formula (I), wherein Y is —CR₁₂═CR₁₃—.

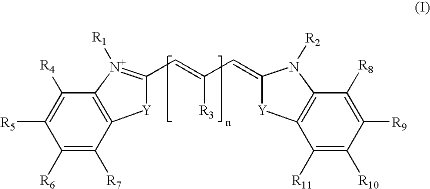

In another embodiment, the dye is a compound according to formula (II), or a salt or ester thereof:

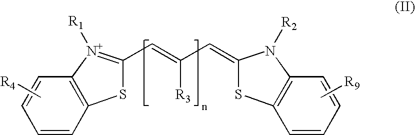

wherein, independently at each occurrence:
R₁ and R₂ are each independently selected from C₁-C₆ alkyl, C₂-C₆ alkenyl, and C₂-C₆ alkynyl,
R₃ is selected from the group consisting of hydrogen, C₁-C₆ alkyl, C₂-C₆ alkenyl, and C₂-C₆ alkynyl, C₆-C₁₀ aryl, hydroxyl, alkoxy, halo, carbonyl, sulfinyl, sulfonyl, and amino groups;
n is 1 or 2;
R₄ and R₉ are each independently selected from the group consisting of hydrogen, C₆-C₁₀ alkyl, C₂-C₆ alkenyl, C₂-C₆ alkynyl, C₁-C₆ aryl, hydroxyl, alkoxy, halo, carbonyl, sulfinyl, sulfonyl, and amino groups.

In a further embodiment, the dye is a compound according to formula (III), or a salt or ester thereof:

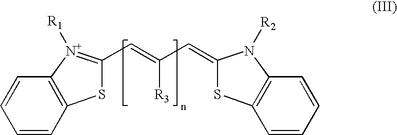

wherein, independently at each occurrence:
R₁ and R₂ are each independently selected from C₁-C₆ alkyl and C₂-C₆ alkenyl;
R₃ is selected from the group consisting of hydrogen and methyl; and n is 1 or 2.

The present invention preferably employs a first composition that includes a dye. More preferably, the first composition includes a dye and a detergent. In another embodiment, the present invention employs a second composition that includes a nucleic acid analog. In yet another embodiment, the present invention employs a third composition that includes a target polynucleotide. Preferably, the third composition is provided for use of the inventive method in a set of containers, each including different concentrations of the target polynucleotide. More preferably, the first composition and the second composition are combined in a separate container. An alternative but also preferred embodiment where the second composition and third composition are combined, preferably in a separate container. Other components used in the methods described herein can also be formulated into suitable compositions and included in separate containers or, as appropriate, combined with one or more of the aforementioned compositions. In certain embodiments, dye is preferably included in all of the compositions; in other embodiments, dye is preferably included in only the first composition; in yet other embodiments, dye is preferably included in a separate container, apart from all other reagents.

The application is further directed to kits for detecting a target polynucleotide. The kits preferably include one or more components used in the methods disclosed herein. In one embodiment, the kit includes one or more nucleic acid analogs that are at least partially complementary to a segment of the target polynucleotide, one or more dyes, and/or one or more detergents. These components can be pre-mixed, as noted herein in setting forth various compositions.

### BRIEF DESCRIPTION OF THE DRAWINGS

FIG. 1 depicts the percent change in fluorescence intensity in mixtures that include 3,3′ diethylthiacarbocyanine iodide dye, ncPNA, target polynucleotide, and one of a series of surfactants.

FIG. 2 depicts the percent change in fluorescence intensity in mixtures that include 3,3′ diethylthiacarbocyanine iodide dye, ncPNA, target polynucleotide, and one of a series of surfactants at 5.0% concentration by volume.

FIG. 3 depicts the percent change in fluorescence intensity in mixtures that include 3,3′ diethylthiacarbocyanine iodide dye, bacterial cells containing target polynucleotide, and one of a series of surfactants at indicated concentration by volume.

FIG. 4 depicts the percent change in fluorescence intensity in mixtures that include 3,3′ diethylthiacarbocyanine iodide dye, target polynucleotide, and one of a series of nucleic acid analogs.

FIG. 5 depicts the percent change in fluorescence intensity in mixtures that include 3,3′ diethylthiacarbocyanine iodide dye, transgenic-specific nucleic acid analog, and polynucleotides from corn leaf lysate of transgenic or wild-type plants.

FIGS. 6A-B depict different schemes of capturing and detecting polynucleotide targets, either on a solid substrate or in a liquid.

FIG. 6C-D depict means of detecting mutations using nucleic acid analogs.

FIG. 7 depicts an agarose gel containing 3,3′-diethylthiacarbocyanine iodide dye in which a non-chiral PNA/target polynucleotide hybrid is resolved.

FIG. 8 depicts a series of single nucleotide polymorphisms detected by the methods disclosed herein.

FIG. 9 depicts a series of two or more nucleotide polymorphisms detected by the methods disclosed herein.

FIG. 10 depicts the change in fluorescence over time using different surfactants in a white plate under lower light intensity.

FIGS. 11A-D depict the change in fluorescence intensity of the “non-chiral PNA:DNA+Dye” reactions and the “Dye Only” reactions using different microtiter plates.

FIG. 12 depicts the reduction in background noise signal from actual signal using a modified phosphate buffer, altered light intensity with a white plate.

FIG. 13 depicts the use of a bacterial permeabilization/lysis buffer on a gram positive bacterial and a gram negative bacterial sample with bacterial specific and non-specific nucleic acid analog probes.

FIG. 14 depicts the use of an altered bacterial permeabilization/lysis buffer and quantitative detection of a bacterial target on a serially diluted culture of bacterial cells.

FIG. 15 depicts the reduction of background noise signal from a specific signal by use of both Tween® 80 and methanol in the buffer compared to methanol alone. Tests were done at reduced light intensities in a white plate.

### DETAILED DESCRIPTION

The present application provides methods, compositions and kits for determining the presence or amount of a target molecule by using nucleic acid analogs and a dye in the context of a reaction mixture that has a characteristic optical property. The target molecule can be any macromolecule or small molecule, as further detailed below. Even when the target molecule is a polynucleotide, the nucleic acid analogs used in the present invention may or may not include a sequence that is complementary to a segment or moiety of the target molecule. The present invention, generally speaking, relates to the presence in a reaction mixture of a hybrid nucleic acid molecule that includes the nucleic acid analog if the target molecule is present; and if so, then the optical property of the reaction mixture changes, thereby indicating the presence of the target molecule.

### I. General Techniques

The practice of the present invention employs, unless otherwise indicated, conventional techniques of molecular biology (including recombinant techniques), microbiology, cell biology, biochemistry, immunology, protein kinetics, and mass spectroscopy, which are within the skill of the art. Such techniques are explained fully in the literature, such as, Sambrook et al., MOLECULAR CLONING: A LABORATORY MANUAL (2d ed., Cold Spring Harbor Press 2000); CELL BIOLOGY: A LABORATORY NOTEBOOK (J. E. Cellis, ed., Academic Press 1998); ANIMAL CELL CULTURE (R. I. Freshney, ed., 1987); METHODS IN ENZYMOLOGY (a series of volumes directed at enzymology protocols that is published by Academic Press, Inc.); HANDBOOK OF EXPERIMENTAL IMMUNOLOGY (D. M. Weir and C. C. Blackwell, eds.); PCR: THE POLYMERASE CHAIN REACTION (Mullis et al., eds., 1994); and the like. Furthermore, procedures employing commercially available assay kits and reagents typically are used according to manufacturer-defined protocols, unless otherwise noted.

### II. Definitions

The term “target molecule” generally refers to a molecule having a nucleic acid sequence or an antigenic determinant or a carbohydrate that is detected using the methods, compositions, or kits disclosed herein. A target molecule can be a macromolecule or a small molecule as those terms are used in the art. In particular, a macromolecule is a polynucleotide, a polypeptide, a carbohydrate, a lipid, or a combination of one or more of these. As a general rule, the molecular mass of a macromolecule is at least about 300 Daltons and can be millions of Daltons. A small molecule is an organic compound having a molecular weight of up to about 300 Daltons.

The term “target nucleic acid sequence” refers to the nucleic acid sequence of a target polynucleotide that hybridizes to a nucleic acid analog or the nucleic acid sequence of a secondary polynucleotide which is at least partially hybridized with the target polynucleotide, for the purpose of detecting the target polynucleotide using the methods, compositions or kits disclosed herein. All or part of the target polynucleotide or secondary polynucleotide which is at least partially hybridized with the target polynucleotide may form a hybrid with a nucleic acid analog by sequence-specific hybridization, albeit some mismatch may exist depending on the conditions of the reaction mixture. The target nucleic acid sequence may be of any length. In certain instances, the target nucleic acid sequence is preferably less than about 1000 bases, less than about 500 bases, less than about 100 bases, less than about 40 bases, or less than about 24 bases. In other embodiments, the target nucleic acid sequence is greater than about 4, about 5, about 6, about 7, about 8, about 9, about 10, about 12, about 14, about 16, about 18, about 20, about 25, about 30, about 35, about 40, about 45, or about 50 bases in length. In yet other embodiments, the target nucleic acid sequence is preferably greater than about 4 bases and less than about 24 bases in length. In certain preferred embodiments, the target nucleic acid sequence is about 4, about 6, about 8, about 10, about 12, bout 14, about 16, about 18, about 20, about 22, or about 24 bases in length. The target nucleic acid sequence may include a protein coding sequence and/or a non-coding sequence (e.g., intergenic spacer sequences regulatory sequences, introns, and the like).

The term “polynucleotide” refers to a polymeric form of nucleotides of any length, either deoxyribonucleotides or ribonucleotides, or analogs thereof. Polynucleotides may be single-stranded, double-stranded, triple-stranded, or multi-stranded to yet greater degrees. The following are non-limiting examples of polynucleotides: a gene or gene fragment, exons, introns, messenger RNA (mRNA), transfer RNA, ribosomal RNA, armored RNA, ribozymes, cDNA, recombinant polynucleotides, branched polynucleotides, plasmids, vectors, nucleic acid probes, primers, amplified DNA, and synthesized DNA. A polynucleotide may contain modified bases, including those that include, without limitation, a methylation, deamination, thiolation, and/or acetylation. The sequence of nucleotides of a polynucleotide may be interrupted by non-nucleotide components, and may include one or more nucleic acid analogs. A polynucleotide may be further modified before or after polymerization, such as by conjugation with a labeling component. The polynucleotide may be an amplified region of a longer sequence of nucleotides.

The term “target polynucleotide” refers to a polynucleotide that includes a target nucleic acid sequence. The target polynucleotide may be of any length. In certain instances, the target polynucleotide is preferably less than about 1000 bases, less than about 500 bases, less than about 100 bases, less than about 40 bases, or less than about 24 bases. In other embodiments, the target polynucleotide is preferably greater than about 8, about 9, about 10, about 12, about 14, about 16, about 18, about 20, about 25, about 30, about 35, about 40, about 45, about 50, about 75, about 100, about 150, about 200, about 250, about 300, about 350, about 400, about 450, about 500, or about 1000 bases in length. In yet other embodiments, the target polynucleotide is preferably greater than about 20 bases and less than about 1000 bases in length; more preferably, greater than about 20 and less than about 500; even more preferably, greater than about 20 and less than about 400 bases in length. In certain preferred embodiments, the target polynucleotide is about 50, about 100, about 150, about 200, about 250, about 300, about 350, about 400, about 450, about 500, or about 1000 bases in length.

The term “secondary polynucleotide” refers to a polynucleotide used in an indirect hybridization method for detecting a target polynucleotide. The secondary polynucleotide includes a portion that is complementary to a target polynucleotide and another portion that is complementary to a nucleic acid or a nucleic acid analog. Preferably, the secondary polynucleotide is employed as a pair. Each of the pair of secondary polynucleotides include, in sequence, (1) a first segment that is complementary to contiguous segments, respectively, of the target polynucleotide, (2) a second segment that is the complement of the analogous portion of the other secondary polynucleotide, and (3) a third segment that is complementary to contiguous segments of a nucleic acid, which may or may not be a nucleic acid analog.

“Armored RNA™” refers to an RNA that is ribonuclease resistant due to the encapsidation of the RNA by bacteriophage proteins. “Armored RNA™” is further described, for example, in U.S. Pat. Nos. 6,399,307; 6,214,982; 5,939,262; 5,919,625; and 5,677,124.

The term “nucleic acid analog” refers to any nucleic acid analog having one or more bases that differ from conventional guanine, thymine, adenosine, cytosine, or uracil, and/or having one or more differences from the conventional phosphoribose of an RNA backbone or the conventional phosphodeoxyribose of a DNA backbone at one or more bases. The nucleic acid analog is preferably greater than about 4 nucleotides in length and less than about 24 nucleic acid bases in length, excluding linkers, amino acids and labels. In other embodiments, the nucleic acid analog may be from about 5 to about 100, from about 8 to about 60, or from about 10 to about 20 nucleic acid bases in length. In another embodiment, the nucleic acid analog is about 6, about 8 about 10, about 12, about 14, about 18, about 22, about 26, about 30, about 35, about 40, or about 45 nucleic acid bases in length, excluding linkers, amino acids and labels. In other embodiments, the target nucleic acid can be at least 4, at least 5, at least 6, at least 7, at least 8, at least 9, at least 10, at least 12, at least 14, at least 15, at least 18, at least 20, at least 25, at least 30, at least 35, at least 40, at least 45, or at least 50 bases in length. Nucleic acid analogs can be chimeric by having a specific type of nucleic acid analog nucleoside in combination with another nucleic acid analog nucleoside, and/or one or more conventional DNA nucleosides or RNA nucleosides.

Exemplary phosphorous modifications useful in creating nucleic acid analogs include chiral phosphorothioate (bridging and non-bridging), phosphorodithioate, chiral methyl phosphonate, chiral phosphoramidate, chiral phosphate trimester, chiral boranophosphate, and chiral phosphoroselenoate. Exemplary linkage modifications include methylenemethylimino (MMI), 3′-amide, 3′ achiral phosphoramidate, 3′ archiral methylene phosphonate, thioformacetal, and thioethyl ether modifications. Examplary sugar modifications include 2′-fluoro, 2′-O-methyl, 2′-O-(3-amino)propyl, 2′-O-(2-methoxy)ethyl, 2′-O-2-(N,N-dimethylaminooxy)ethyl (DMAOE), 2′-O-2-[2-(N,N-dimethylamino)ethyloxy]ethyl (DMAEOE), and 2′-O-N,N-dimethylacetamidyl. Classes of analog nucleotides having sugar modifications include N-morpholinophosphordiamidate (Morpholinos); hexose nucleic acid (HNA); threose nucleic acid (TNA), such as those disclosed in Chaput et al., AMER. CHEM. SOC., 125:856-857 (2003); cyclohexene nucleic acid (CeNA); locked nucleic acid (LNA), having methylene bridges between the 2′-O and 4′-C on the ribofuranose ring of some or all individual nucleotides of a polynucleotide (which methylene bridges function to restrict the flexibility of the polynucleotide and are associated with enhanced stability and hybridization characteristics), such as those disclosed in TRENDS IN BIOTECHNOLOGY 21:74-81 (2003); and tricycle-deoxyribose nucleic acid (tcDNA) modifications. Preferred base modifications include 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, and phenoxazinyl-G-clamp. A preferred connection modification is an α-deoxyribofuranosyl. Preferred sugar replacement modifications include production of a peptide nucleic acid (PNA) or a chiral peptide nucleic acid or a non-chiral PNA. Other exemplary nucleic acid analogs include sequence-specific DNA binding minor groove ligands, such as polyamides (containing imidazole (Im), pyrrole (Py), and hydroxypyrrole (Hp)). Nucleic acid analogs can be chimeric, and have multiple different modifications, and can include non-nucleic acid analogs, such as linkers, as are known in the art. Polynucleotides that include analog nucleotides are described as to sequence with respect to its non-modified analog as identified in the accompanying Sequence Listing, with further description of which, if not all, included nucleotides are modified.

The term “photochemical reaction” refers to a reaction that can occur when electromagnetic radiation interacts with matter and initiates the production of new chemical species. Absorption of electromagnetic radiation, typically in the region of the electromagnetic spectrum which ranges from approximately 180 nanometers in the ultraviolet to 800 nanometers in the near infrared, initiate electronic transitions in the absorbing species and result in a temporary change in its electronic structure. This electronically excited species may reemit the energy absorbed via radiationless decay, fluorescent emission, or phosphorescent emission resulting in no change to the original absorber. Alternatively, the electronically activated species can undergo an irreversible electronic change creating a new product molecule or molecules. Also, the electronically excited species can interact with a second molecule with different chemical structure in the sample causing changes in that molecules electronic structure which in turn can cause reversible or irreversible changes to the second molecule. Products of these photochemically induced reactions can in-turn react with other chemically distinct molecules in the sample to initiate other chemical reactions.

The term “reaction mixture” refers to the mixture of at least the following when the nucleic acid analog is the complement of a target nucleic acid sequence of the target molecule: (1) a target polypeptide; (2) a nucleic acid analog or a target binding component; and (3) a dye. Alternatively, when the nucleic acid analog and its complement are unrelated to the target polypeptide, and instead are attached to a target binding component as part of the reporter complex, the reaction mixture comprises: (1) a target molecule; (2) a target binding component; and (3) a dye.

The term “peptide nucleic acid,” or “PNA,” includes any nucleic acid analog in which the deoxyribose phosphate backbone of a nucleic acid has been replaced by a synthetic peptide-like backbone, including, for example, n-(2-amino-ethyl)-glycine units, such as, without limitation, those disclosed in U.S. Pat. Nos. 5,786,461; 6,357,163; 6,107,470; 5,773,571; 6,441,130, 6,451,968; 6,228,982; 5,641,625; 5,766,855; 5,736,336; 5,719,262; 5,714,331; 5,719,262; and 6,414,112. The purine and pyrimidine bases may be attached by any covalent linkage, including, for example, methylene carbonyl linkages. As used herein, PNA molecules can have additional atoms between the PNA backbone and nucleobase. These analogs include, for example, D-lysine chains, cyclic structures, such as cyclopentane or pyrrolidine rings, and/or chiral substituents, including PNA molecules described in U.S. Pat. No. 6,403,763, U.S. Patent Application US 2003/0162699, and U.S. Patent Application US 2003/0157500. The PNA backbone may include substitutions or extensions in the peptide backbone. PNAs may include peptide-based nucleic acid mimics (PENAMS), such as those disclosed, for example, in U.S. Pat. No. 5,705,333, atoms having unusual chiral centers, such as D-chiral centers and quasi-chiral centers, and atom substitutions in the PNA backbone.

The term “chiral PNA” or “cPNA” refers to a chiral PNA molecule in which at least a portion of the peptide backbone has been modified to include a proline or modified proline side-chain that includes the backbone nitrogen and α-carbon. Non-limiting examples of chiral PNA molecules include those that are disclosed at, for example, U.S. Pat. No. 6,403,763, U.S. Patent Applications US 2003/0162699 and US 2003/0157500.

The term “achiral PNA” or “non-chiral PNA” or “ncPNA” refers to a PNA molecule in which no portion of the peptide backbone has been modified to include a proline or modified proline side chain that includes the backbone nitrogen and α-carbon.

The term “non-PNA nucleic acid analog” refers to a nucleic acid analog in which the backbone is not made up of n-(2-amino-ethyl)-glycine subunits.

The term “locked nucleic acid” or “LNA” refers to a bicyclic nucleic acid in which at least one ribonucleoside is linked between the 2′-oxygen and the 4′-carbon with a methylene group. Non-limiting examples of LNAs are disclosed in TRENDS IN BIOTECHNOLOGY 71:74-81 (2003).

The term “morpholino nucleic acid” or “MNA” refers to a nucleic acid analog in which each backbone monomer is a substituted or unsubstituted six-membered morpholino ring. The morpholino rings are linked by non-ionic phosphorodiamidate linkages. Non-limiting examples of MNAs include those described in U.S. Pat. No. 5,034,506.

The term “threose nucleic acid” or “TNA” refers to a nucleic acid in which the sugar-phosphate backbone is a four-carbon sugar threose in place of the five-carbon sugar ribose.

The term “metal linked nucleic acid” or “MLNA” refers to a nucleic acid sequence in which at least a portion of the ribose phosphate backbone is modified with a transition metal. Non-limiting examples of MLNAs include those MLNAs disclosed at the website of the Wilker Research Group, Purdue University website. An exemplary MLNA is a metal linked 2′-O-Methyl oligoribonucleotide.

The terms “nucleic acid analog/polynucleotide hybrid” and “polynucleotide/nucleic acid analog hybrid” and “NAA/TP hybrid” are synonymous and refer to a nucleic acid analog and target polynucleotide hybridized in a sequence-specific manner. Non-limiting examples of nucleic acid analog/polynucleotide hybrids include nucleic acid analog/polynucleotide duplexes and triplexes.

The terms “PNA/polynucleotide hybrid” and “polynucleotide/PNA hybrid” are synonymous and refer to a PNA and polynucleotide hybridized in a sequence-specific manner. Non-limiting examples of PNA/polynucleotide hybrids include PNA/polynucleotide duplexes and triplexes. The PNA may be chiral or non-chiral.

By “complementary”, it is meant that a single-stranded nucleic acid analog has the ability to bind a polynucleotide in a base-specific manner. The nucleic acid analog may be synthesized to bind a target polynucleotide, such as a full-length polynucleotide strand or a part thereof. A nucleic acid analog that is “complementary” may have one or more single base-pair mismatches, additions, and/or deletions, but is still capable of hybridizing to the target polynucleotide under the selected hybridization conditions. In one embodiment, complementary sequences may hybridize through Watson-Crick base pairing (A-T or A-U and C-G or alternatively pairing with inosine). In a further embodiment, complementary sequences may hybridize through Hoogstein base pairing.

By “exactly complementary”, it is meant that the single-stranded nucleic acid analog has the ability to hybridize to a target nucleic acid sequence without base mismatches. A nucleic acid analog is not exactly complementary to a target polynucleotide if there is a single base-pair mismatch between the nucleic acid analog and the target polynucleotide.

The term “rate” refers to a change (e.g., of a property of a composition or compound). A rate may be described in terms of a specific rate constant. A rate may be determined by making measurements over a period of time. A rate may be described by making measurements, determined by measurements at two different time points in a process or by making measurements at least three, at least four, or at least five, timepoints. A rate may be determined based on a single measurement and a known quantity, such as a previously known or calculated quantity. A rate may be expressed in quantitative or qualitative terms (e.g., a change is “fast” or “slow”). A rate may be determined by comparing a property or compound to a reference value, or by observation of changes in a given property or compound over time, using standard methods.

As used herein, the term “relative rate” refers to the rate of one process compared to the rate of another process. A “relative rate” may be approximate (e.g., the rate of one process may be “faster” or “slower” than the rate of another process) or quantitative (e.g., comparing measured rate constants of two processes).

As used herein, the term “dye” refers to a first compound that has a measurable optical property or that may be converted to a second compound with a measurable optical property. Preferred dyes include those where the measurable optical property thereof differs in comparison to that of the second compound. Measurable optical properties include, but are not limited to color, absorbance, fluorescence, reflectance, chemiluminescence, and infrared (IR) spectrum measurements. The dye may exhibit the optical property under certain conditions, such as binding or forming a complex or otherwise being in contact with a target polynucleotide/nucleic acid analog hybrid or, or not binding or forming a complex or otherwise being in contact with a target polynucleotide/nucleic acid analog hybrid.

A “hybrid catalyst” refers to a hybrid molecule that is capable of promoting the photodegradation of a dye. The following examples are usefully employed in the present invention: nucleic acid-nucleic acid hybrids, nucleic acid analog-nucleic acid hybrids, nucleic acid analog-nucleic acid analog hybrids. The present invention establishes reaction conditions by which the nucleic acid-nucleic acid hybrids present in any cell lysate, for example, contribute minimally to the catalytic function assigned to any of these hybrids. The same conditions that minimize the ability of the nucleic acid-nucleic acid hybrids to change an optical property also tend to potentiate the activity of the nucleic acid-nucleic acid analog hybrids.

“Sample” refers to a liquid sample of any type (e.g., blood, serum, water, urine, fecal matter, sputum, or lysate or extract of a solid sample), a solid sample of any type (e.g., cells, food, ice, dirt, grain, or material acquired from a surface), an airborne sample of any type, and/or a material embedded in a gel material and/or any solid-phase material, such as agarose, acrylamide, or gelatin.

The term “pathogen” refers to any agent causing a disease, disorder and/or pathological condition and/or symptoms. By way of example, the pathogen may be an organism (or its associated toxin) found in nature, or created in a laboratory, that causes disease in or development of a pathological condition or symptom in, incapacitates, debilitates and/or kills an organism. Pathogens include, but are not limited to, viruses, bacteria, fungi, eukaryotes, and/or prokaryotes; and may function as biological weapons agents, or vectors of infectious diseases; and may spread via water, as in water-borne pathogens, food, as in food pathogens, air or direct skin contact.

The term “biological weapons agent” refers to any organism (or its associated toxin) found in nature or created in the laboratory that is used for the primary purpose of causing disease in, incapacitating, or killing another living organism. Examples of biological weapons agents include, but are not limited to, pathogenic bacteria, fungi, protozoa, rickettsiae, and viruses. The target of a biological weapons agent includes any of humans, animals, and plants, as well as sub-populations thereof.

As used herein, the term “infection” refers to the presence of a pathogen in or on a host. The infection may be dormant or virulent. In one embodiment, the presence of the pathogen is indicated by an alteration in host polynucleotide and/or polypeptide expression. Infection may occur through such routes including, but not limited to, airborne droplets, direct contact, animal or insect vectors, and contaminated food or drink.

As used herein, the term “host-response polynucleotide” refers to a polynucleotide that is altered, or a polynucleotide for which the expression is altered, in a host in response to a stimulus, such as infection, and/or contact by a pathogen.

The term “host” as used herein refers to humans, animals, and plants. The animal may be a mammal. Examples of animal mammals include, non-human primates, farm animals, sport animals, mice, and rats. Examples of plants include, but are not limited to, dicot or monocot agricultural crops.

As used herein, the term “alkyl,” “alkenyl,” and “alkynyl” refer to straight-chain, branched-chain and cyclic monovalent substituents, and can be substituted or unsubstituted. Examples include methyl, ethyl, isobutyl, cyclohexyl, cyclopentylethyl, 2-propenyl, 3-butynyl, and the like. Typically, the alkyl, alkenyl and alkynyl substituents contain C₁₋₁₀ (alkyl) or C₂₋₁₀ (alkenyl or alkynyl). Preferably they contain C₁₋₆ (lower alkyl) or C₂₋₆ (lower alkenyl or lower alkynyl). Examples of alkyl groups include propyl, tert-butyl, and cycloalkyls such as cyclopropyl, cyclobutyl, cyclopentyl, cyclohexyl, and cycloheptyl groups. Examples of alkenyl groups include allyl, crotyl, 2-pentenyl, 3-hexenyl, 2-cyclopentenyl, 2-cyclohexenyl, 2-cyclopentenylmethyl, and 2-cyclohexenylmethyl groups.

As used herein, the terms “heteroalkyl,” “heteroalkenyl,” and “heteroalkynyl” are similarly defined but may contain one or more O, S or N heteroatoms or combinations thereof within the backbone residue; collectively, the aforementioned terms having the “hetero-” prefix are referred to as “hetero forms.”

As used herein, “acyl” encompasses the definitions of alkyl, alkenyl, alkynyl, each of which is coupled to an additional residue through a carbonyl group. Heteroacyl includes the related heteroforms.

“Aromatic” moiety or “aryl” moiety refers to a monocyclic or fused bicyclic moiety such as phenyl or naphthyl; “heteroaryl” refers to monocyclic or fused bicyclic ring systems containing one or more heteroatoms selected from O, S and N. The inclusion of a heteroatom permits inclusion of 5-membered rings as well as 6-membered rings. Thus, typical aryl/heteroaryl systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl, and the like. Because tautomers are theoretically possible, phthalimido is also considered aromatic. Any monocyclic or fused ring bicyclic system that has the characteristics of aromaticity in terms of electron distribution throughout the ring system is included in this definition. Typically, the ring systems contain 5- to 12-ring-member atoms.

Similarly, “arylalkyl” and “heteroarylalkyl” refer to aryl and heteroaryl systems that are coupled to another residue through a carbon chain, including those carbon chains that are substituted or unsubstituted, saturated or unsaturated, typically having one to eight carbon atoms, including hetero forms thereof. These carbon chains may also include a carbonyl group, thus making them able to provide substituents as an acyl or heteroacyl moiety.

In general, any alkyl, alkenyl, alkynyl, acyl, or aryl group contained in a substituent may itself optionally be substituted by additional substituents. The nature of these substituents is similar to those recited with regard to the primary substituents themselves. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments. However, alkyl substituted by aryl, amino, alkoxy, and the like would be included.

As used herein, the term “halogen” and “halo” are used interchangeably and refer to one or more substituents including fluorine, chlorine, bromine, iodine, and astatine.

Examples of substituted hydroxyl and thiol groups include substituted alkyloxy or alkylthio (e.g., C₁₋₁₀ alkyl), such as methyl, ethyl, propyl, isopropyl, butyl, isobutyl, sec-butyl, tert-butyl, pentyl, cyclopropyl, cyclobutyl, cyclopentyl, cyclohexyl, and cycloheptyl, a substituted arylalkyloxy or arylalkylthio (e.g., phenyl-C₁₋₄ alkyl, benzyl, or phenethyl). Where there are two adjacent hydroxyl or thiol substituents, the heteroatoms may be connected via an alkylene group such as O(CH₂)ₙO and S(CH₂)ₙS (where n=1-5).

Examples of substituted hydroxyl groups also include optionally substituted C₂₋₄ alkanoyl (e.g., acetyl, propionyl, butyryl or isobutyryl), C₁₋₄ alkylsufonyl (e.g., methanesulfonyl or ethanesulfonyl) and a substituted aromatic and heteroaryl carbonyl group, including benzoyl and pyridinecarbonyl.

Substituents on substituted amino groups may bind to each other to form a cyclic amino group (e.g., 5- to 6-membered cyclic amino, etc., such as tetrahydropyrrole, piperazine, piperidine, pyrrolidine, morpholine, thiomorpholine, pyrrole or imidazole). The cyclic amino group may have a substituent, and examples of the substituents include halogen, nitro, cyano, hydroxy group, thiol group, amino group, carboxyl group, an optionally halogenated C₁₋₄ alkyl, an optionally halogenated C₁₋₄ alkoxy (e.g., methoxy, ethoxy, trifluoromethoxy, trifluoroethoxy, etc.), C₂₋₄ alkanoyl (e.g., acetyl or propionyl), and C₁₋₄ alkylsulfonyl (e.g., methanesulfonyl or ethanesulfonyl).

An amino group may also be substituted once or twice (to form a secondary or tertiary amine) with a group such as an optionally substituted alkyl group including C₁₋₁₀ alkyl (e.g., methyl or ethyl propyl); an optionally substituted alkenyl group, such as allyl, crotyl, 2-pentenyl, 3-hexenyl, and the like, or an optionally substituted cycloalkyl group such as cyclopropyl, cyclobutyl, cyclopentyl, cyclohexyl or cycloheptyl. In certain cases, these groups are C₁₋₆ alkyl, C₂₋₆ alkenyl, or cycloalkyl groups. The amine group may also be optionally substituted with an aromatic or heteroaromatic group, aralkyl (e.g., phenyl C₁₋₄ alkyl) or heteroalkyl, for example, phenyl, pyridine, phenylmethyl (benzyl), phenethyl, pyridinylmethyl, or pyridinylethyl. The heteroaromatic group may be a 5- or 6-membered ring containing 1-4 heteroatoms.

An amino group may be substituted with an optionally substituted C₂₋₄ alkanoyl (e.g., acetyl, propionyl, butyryl, and isobutyryl), or a C₁₋₄ alkylsulfonyl (e.g., methanesulfonyl or ethanesulfonyl), or a carbonyl- or sulfonyl-substituted aromatic or heteroaromatic ring (e.g., benzenesulfonyl, benzoyl, pyridinesulfonyl and pyridinecarbonyl). The heteroaromatics are as defined above.

Examples of carbonyl groups, sulfinyl groups, or sulfonyl groups include substituted or unsubstituted forms of such groups formed from various hydrocarbyls, such as alkyl, alkenyl and 5- to 6-membered monocyclic aromatic group (e.g., phenyl and pyridyl), as defined above.

The term “salt” is meant to include salts of the active compounds from any acid or base known in the art, as appropriate to the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Examples of acid addition salts include (1) any halogen; (2) those derived from an inorganic acid, such as hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acid, or the like; and those derived from relatively nontoxic organic acids, such as acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids, including arginate and the like, and salts of organic acids, including glucuronic or galacturonic acid and the like (see, for example, S. M. Berge et al., J. PHARMA. SCI. 66:1-19 (1977)). Certain specific compounds of the present application contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents. Otherwise, however, the salts are equivalent to the parent form of the compound for the purposes of the present application.

As used herein, the term “target binding component” refers to a molecule capable of interacting with a target molecule. Target binding components having limited cross-reactivity are generally preferred. In certain embodiments, suitable target binding components include, for example: lectins; receptors; antibodies, including monoclonal antibodies, polyclonal antibodies, and derivatives or analogs thereof, including without limitation, Fv fragments, single chain Fv (scFv) fragments, Fab′ fragments, F(ab′)₂ fragments, single domain antibodies, camelized antibodies and fragments thereof, humanized antibodies and fragments thereof, and multivalent versions of the foregoing. Multivalent versions of target binding components include without limitation: monospecific or bispecific antibodies, such as disulfide stabilized Fv fragments, scFv tandems ((scFV)₂ fragments), diabodies, tribodies or tetrabodies, which typically are covalently linked or otherwise stabilized (i.e., leucine zipper or helix stabilized) scFv fragments. Other binding reagents include, for example, template imprinted materials (such as those of U.S. Pat. No. 6,131,580), and organic or inorganic binding elements. In preferred embodiments, a target binding component specifically interacts with a single identifying unit of the target molecule, such as an epitope or a sugar or a ligand. In other embodiments, a target binding component may interact with several structurally related epitopes or sugars or ligands.

The term “reporter complex” refers to a first reporter nucleotide sequence, a second reporter nucleotide sequence, and a dye. The first reporter nucleotide sequence and second reporter nucleotide sequence can be covalently bonded together, or not covalently bonded together.

The term “modified target binding component” refers to a target binding component that is covalently modified by at least one component of the reporter complex.

The term “target binding complex” refers to the modified target binding component and the remaining components of the reporter complex.

The term “antibody” refers to an immunoglobulin, derivatives thereof that maintain specific binding ability, and proteins having a binding domain that is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. In preferred embodiments, antibodies used with the methods and compositions described herein are derivatives of the IgG class.

The term “antibody fragment” refers to any derivative of an antibody that is less than full-length. In preferred embodiments, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially produced synthetically. The antibody fragment may optionally be a single chain antibody fragment. Alternatively, the fragment may comprise multiple chains that are linked together, for instance, by disulfide linkages. The fragment may also optionally be a multimolecular complex. A functional antibody fragment will typically comprise at least about 50 amino acids and more typically will comprise at least about 200 amino acids.

As used herein, the term “array” refers to a set of target binding components immobilized onto one or more substrates so that each target binding component is at a known location. Alternatively, the set of target binding components may be in solution, respectively in different receptacles of a microtiter dish, and therefore, located at known locations. In a preferred embodiment, a set of target binding components is immobilized onto a surface in a spatially addressable manner so that each individual target binding component is located at different and identifiable location on the substrate.

The term “camelized antibody” refers to an antibody or variant thereof that has been modified to increase its solubility and/or reduce aggregation or precipitation, similar to that found in a camelid. Camelids produce heavy-chain antibodies consisting only of a pair of heavy chains wherein the antigen binding site comprises the N-terminal variable region or VEH (variable domain of a heavychain antibody). The VHH domain comprises an increased number of hydrophilic amino acid residues that enhance the solubility of a VHH domain as compared to a VH region from noncamelid antibodies. Camelization of an antibody or variant thereof preferably involves replacing one or more amino acid residues of a non-camelid antibody with corresponding amino residues from a camelid antibody.

The term “chemical handle” refers to a component that may be attached to a target binding complex as described herein so as to facilitate the isolation, immobilization, identification, detection and/or increased solubility of the target binding complex. Suitable chemical handles include, for example, a polypeptide, a polynucleotide, a carbohydrate, a polymer, or a chemical moiety, and combinations or variants thereof.

The term “diabodies” refers to dimeric scFvs. The components of diabodies preferably have shorter peptide linkers than most scFvs and they show a preference for associating as dimers. The term diabody is intended to encompass both bivalent (i.e., a dimer of two scFvs having the same specificity) and bispecific (i.e., a dimer of two scFvs having different specificities) molecules. Methods for preparing diabodies are known in the art. See, e.g., EP 404097 and WO 93/11161.

As used herein, the term “epitope” refers to a physical structure on a molecule that interacts with a target binding component. In exemplary embodiments, epitope refers to a desired region on a target molecule that specifically interacts with a target binding component.

The term “Fab” refers to an antibody fragment that is substantially equivalent to that obtained by (1) digestion of immunoglobulin (typically IgG) with the enzyme papain or (2) reduction of the disulfide bridge or bridges joining the two heavy chain pieces in the F(ab′)₂ fragment. The heavy chain segment of the Fab fragment is the Fd piece. Such fragments may be enzymatically or chemically produced by fragmentation of an intact antibody, recombinantly produced from a gene encoding the partial antibody sequence, or wholly or partially synthetically produced. Methods for preparing Fab fragments are known in the art. See, e.g., Tijssen, PRACTICE AND THEORY OF ENZYME IMMUNOASSAYS (Elsevier, Amsterdam, 1985).

The term “F(ab′)₂” refers to an antibody fragment that is substantially equivalent to a fragment obtained by digestion of an immunoglobulin (typically IgG) with the enzyme pepsin at pH 4.4. Such fragments may be enzymatically or chemically produced by fragmentation of an intact antibody, recombinantly produced from a gene encoding the partial antibody sequence, or wholly or partially synthetically produced.

The term “Fv” refers to an antibody fragment that consists of one VH and one VL domain held together by noncovalent interactions. The term “dsFv” is used herein to refer to an Fv with an engineered intermolecular disulfide bond to stabilize the VH-VL pair. Methods for preparing Fv fragments are known in the art. See, e.g., Moore et al., U.S. Pat. No. 4,462,334; Hochman et al., BIOCHEMISTRY 12:1130 (1973); Sharon et al., BIOCHEMISTRY 15:1591 (1976); and Ehrilch et al., U.S. Pat. No. 4,355,023.

The term “immunogen” refers to compounds that are used to elicit an immune response in a human or an animal, and is used as such herein. Many techniques used to produce a desired target binding component, such as the phage display methods described below, do not rely wholly, or even in part, on immunizations. Nevertheless, these methods use compounds containing an “epitope,” as defined above, to select for and clonally expand a population of target binding components specific to the “epitope.” These in vitro methods mimic the selection and clonal expansion of immune cells in vivo. Therefore, the compounds containing the “epitope” that is used to clonally expand a desired population of phage and the like in vitro are embraced within the definition of “immunogens.” Similarly, the terms “hapten” and “carrier” have specific meaning in relation to standard immunization protocols. In that context, and as used herein, a “hapten” is preferably a small molecule that contains an epitope, but is incapable of serving as an immunogen by itself. Therefore, to elicit an immune response to the hapten, the hapten is preferably conjugated with a larger carrier, such as bovine serum albumin or keyhole limpet hemocyanin, to produce an immunogen. A preferred immune response would recognize the epitope on the hapten, but would not recognize any epitopes that may be on the carrier.

In the in vitro methods described herein for preparing the desired binding reagents, traditional “haptens” and “carriers” typically have their counterpart in epitope-containing compounds affixed to suitable substrates or surfaces, such as beads and tissue culture plates.

The terms “single-chain Fvs” and “scFvs” refers to recombinant antibody fragments consisting of only the variable light chain (VL) and variable heavy chain (VH) covalently connected to one another by a polypeptide linker. Either VL or VH may be the NH2-terminal domain. The polypeptide linker may be of variable length and composition so long as the two variable domains are bridged without function-defeating steric interference. In exemplary embodiments, the linkers are comprised primarily of stretches of glycine and serine residues with some glutamic acid or lysine residues interspersed for solubility. Methods for preparing scFvs are known in the art. See, e.g., PCT/TJS/87/02208 and U.S. Pat. No. 4,704,692.

The term “single domain antibody” or Fd refers to an antibody fragment comprising a VH domain that interacts with a given antigen. An Fd does not contain a VL domain, but may contain other antigen-binding domains known to exist in antibodies, such as the kappa and lambda domains. Methods for preparing Fds are known in the art. See, e.g., Ward et al., NATURE 341:644-646 (1989) and EP 0368684.

The term “single chain antibody” refers to an antibody fragment that comprises variable regions of the light and heavy chains joined by a flexible linker moiety. Methods for preparing single chain antibodies are known in the art. See, for example, U.S. Pat. No. 4,946,778 to Ladner et al.

The term “triabody” refers to trivalent constructs comprising 3 scFv's, and thus comprising 3 variable domains (see, e.g., Iliades et al., FEBS LETT. 409(3):437-41 (1997)). This term also refers to molecules that comprise three variable domains having the same specificity, or three variable domains wherein two or more of the variable domains have different specificities.

The term “tetrabody” refers to engineered antibody constructs comprising four variable domains (see, e.g., Pack et al., J. MOL. BIOL. 246:28-34 (1995) and Coloma and Morrison, NAT. BIOTECHNOL. 15:159-63 (1997)). This term also refers to molecules that comprise four variable domains having the same specificity, or four variable domains wherein two or more of the variable domains have different specificities.

The term “VH” refers to a heavy chain variable region of an antibody.

The term “VL” refers to a light chain variable region of an antibody.

The term “optical property” refers to an intrinsic property of a material that can be observed when the material interacts with electromagnetic radiation in the region of the electromagnetic spectrum that ranges from approximately 180 nanometers in the ultraviolet to 40 micrometers in the infrared. Observable optical properties include absorption of specific wavelengths of electromagnetic radiation, or absorption of specific wavelengths of electromagnetic radiation followed by emission at other specific wavelengths. Measuring such optical properties is well known in the art and uses commercially-available UV-VIS spectrometers and fluorophotometers. In the particular case of chiral molecules, the absorption of polarized electromagnetic radiation may also be measured by techniques such as Polarimetry and Circular Dichroism.

Observable optical properties can also include chemical changes that happen to the sample when it absorbs electromagnetic radiation. Absorption of electromagnetic radiation may initiate an electronic rearrangement in the absorbing species and result in a change in its chemical reactivity or structure, or this electronically-activated species may interact with another molecule in the sample causing its structure or reactivity to change. In either of these cases, changes could occur in the absorption or emission spectra of the sample. Also, such changes could result in the appearance or disappearance of one of the materials in the reaction mixture. Such changes could therefore be measured by any of the aforementioned photometric methods.

### III. Further Summarization of the Embodiments

The present invention relates in a first embodiment to a method of detecting a target polynucleotide in a sample. Preferably, the method includes producing a reaction mixture comprising the sample, a first nucleic acid or a first nucleic acid analog, and a dye. Preferably, the first nucleic acid or the first nucleic acid analog is at least partially complementary to a segment of the target polynucleotide. Another step of the method involves exposing the reaction mixture to a light, which light serves to activate the reaction that causes the change in the optical property of the reaction mixture. Yet another aspect of the method involves observing the optical property of the reaction mixture at least once after exposure to the light. Preferably, the reaction mixture has an optical property that changes in response to the light exposure if the first nucleic acid or first nucleic acid analog and the target polynucleotide are present therein, and wherein the dye is the compound of formula (I), or a salt or ester thereof, as described elsewhere in this specification. The present invention further provides for correlating the detecting of the target polynucleotide with the resultant change in the optical property of the reaction mixture.

Although applicants are not asserting any particular theory or mechanism by which the method set forth here works, evidence has been gained regarding the chemical reaction of oxidation that appears to be associated with at least one of the dyes usefully employed in the diagnostic test. Accordingly, the change in the optical property noted as a reporter of certain recognition-based activity of nucleic acids and binding pair type molecules correlates with a chemical change in the dye. Other mechanisms may be in play in the alternative or in addition. Suffice to say that when the aforementioned components of a reaction mixture are combined followed by a light exposure for activation, the optical property is commonly seen with a wide number of cyanine dyes.

A part of the present invention is the observation of the reaction mixture to determine whether the optical property has changed. Preferably, one will observe the reaction mixture at least twice.

In a preferred embodiment, the optical property includes a first optical property that diminishes after exposure to the light and a second optical property that increases after exposure to the light. Typically, the property that diminishes is inherent to the reaction mixture, and reliably occurs to the extent that the reaction mixture includes a nucleic acid hybrid and dye, where the nucleic acid hybrid is composed of any pair-wise combination of a nucleic acids and nucleic acid analogs. Upon the diminishing optical property, the reaction mixture, contained in a vessel, further comprises a substance that delivers the second optical property; alternatively, the second optical property is contributed by a substance applied to the vessel itself, for example. The substance that contributes this second optical property preferably is not itself a dye.

In another preferred embodiment, the reaction mixture includes a detergent. The detergent has multiple functions, including stabilizing the dye, apparently, and lysing cells for study. Where a sample includes intact cells prior to being in contact with the detergent, the cell lyses and renders its genetic material, for example, available for study.

The reaction mixture in a particularly preferred embodiment is directed at a reaction mixture that preferably includes an achiral peptide nucleic acid. A similarly preferred alternative are those reaction mixtures that include a chiral peptide nucleic acid.

In a preferred embodiment, the length of the target polynucleotide is greater than about 50 bases.

In another preferred embodiment, the reaction mixture further comprises a second nucleic acid, wherein at least a portion of the second nucleic acid is complementary to a portion of the first nucleic acid that is not complementary to the target polynucleotide. This preferred reaction mixture further comprises a third nucleic acid, and wherein one portion of the third nucleic acid is complementary to a portion of the target polynucleotide that is not complementary to the first nucleic acid and wherein another portion of the third nucleic acid is complementary to a portion of the second nucleic acid that is not complementary to the first nucleic acid. Preferably, the first part and the second part of the nucleic acid analog do not overlap.

The nucleic acid analog used in the context of the present invention is greater than about 4 bases in length and less than about 24 bases in length. In another embodiment, the nucleic acid analog is about 12 nucleic acid bases in length.

The present method also includes immobilizing the target polynucleotide and the nucleic acid analog on a solid substrate. In a preferred aspect, the nucleic acid analog is attached to a solid substrate.

In a preferred embodiment, the dye is a compound of formula (I), wherein R₁, R₂, and R₃ are hydrogen or hydrophobic alkyls, R₄ through R₁₃ are hydrogen, and Y is sulfur. In a second embodiment, the dye is of formula (I), wherein n is 1. More preferably, the dye is of formula (I), wherein n is 1; and wherein Y is sulfur or —CR₁₂═CR₁₃—. Even more preferably, the dye is of formula (I), wherein n is 1; wherein Y is sulfur or —CR₁₂═CR₁₃—; and wherein R₁ and R₂ are each independently selected from the group consisting of alkyl and alkenyl.

Yet another embodiment relates to a method of detecting a target polynucleotide in a sample, which includes producing a reaction mixture comprising the sample, a nucleic acid analog that is complementary to a target nucleic acid sequence of the target polynucleotide, and a dye; exposing the reaction mixture to a light; and observing the absorbance of the reaction mixture at least once. Preferably, the reaction mixture has an absorbance that changes if the target polynucleotide and the nucleic acid analog form a hybrid therein. Also preferably, the dye is the compound of formula (I), as set forth elsewhere in this specification. This embodiment further includes correlating the detecting of the target polynucleotide with the resultant change in the optical property of the reaction mixture. What is observed is the potential change in the optical property of the reaction mixture, which correlates with a chemical change in the dye.

The present invention also relates to a composition that includes a surfactant and a dye according to formula (I) or formula (II) or formula (III), or a salt thereof, as set forth elsewhere here.

The inventive composition can further include a nucleic acid analog; or target polynucleotide.

Yet another preferred embodiment is a kit for detecting a target polynucleotide, which includes one or more nucleic acid analogs at least partially complementary to a target nucleic acid sequence of said target polynucleotide; one or more dyes; one or more surfactants; and instructions that relate to the method set forth herein above.

Another preferred embodiment is a reporter complex that includes a first polynucleotide, a second polynucleotide, and a dye, wherein the first polynucleotide and the second polynucleotide form a hybrid and the reporter complex has an optical property that changes in response to exposure to a light stimulus. This reporter complex is used in with respect the optical property is absorbance. The hybrid used in the reporter is attached to a target binding component, which target binding component is selected from the group consisting of an antibody or fragment thereof, a lectin, or a receptor, among other selective binding agents. For example, aptamers, molecular imprints, and avimers may all be used in the context of the present invention. In a particularly preferred use, the polypeptide and the nucleic acid analog are immobilized on a solid substate. Preferably, the second polynucleotide is a nucleic acid analog.

The present invention also relates to a method for detecting a target molecule in a sample, in which the target molecule and a target binding component bind one another with substantial specificicity including combining the sample, the target binding component, a first polynucleotide, and a second polynucleotide in a reaction mixture in a vessel. Preferably, the first polynucleotide and second polynucleotide form a hybrid and are in contact with the target binding component. Also preferably, at least one of the target binding component, first polynucleotide, and second polynucleotide, or the target molecule are attached to a solid surface. The method also includes washing the reaction mixture; and combining the reaction mixture components that are immobilized on the solid substrate with dye, whereupon the reaction mixture has an optical property that changes if the sample includes the target molecule and it and the target binding component bind one another. A further aspect includes exposing the reaction mixture to light; and observing the optical property of the reaction mixture at least once. Again, this method is used with one of the dyes disclosed elsewhere, namely a compound of formula (I).

A particularly preferred aspect of the present invention is a catalytic hybrid that includes two polynucleotides or a polynucleotide and a nucleic acid analog or two nucleic acid analogs that together form the hybrid, wherein the hybrid catalyses a chemical reaction of a dye upon exposure to a light stimulus. The dye that the catalytic hybrid acts upon has been well-described here, namely a compound of formula (I).

For each of the methods disclosed here, the quantity of the target polynucleotide is determined by comparing the observed optical property as compared to a reference.

### IV. Methods of Detecting Polynucleotides

The present invention relates to methods, compositions and kits for determining the presence or amount of a target polynucleotide having a target nucleic acid sequence. In one embodiment, (i) a sample for testing for the presence or amount of a target polynucleotide, (ii) a nucleic acid analog that binds a target nucleic acid sequence of the target polynucleotide in a sequence-specific manner, and (iii) a dye are combined to produce a reaction mixture that has an observable optical property that can change over time. If the target polynucleotide is present, then it and the nucleic acid analog will form a hybrid (referred to herein as the “NAA/TP” hybrid). The rate of change in the optical property of the mixture is preferably different in the presence and absence of the NAA/TP hybrid. Optionally, a light stimulus is applied to the mixture; in a preferred embodiment, the light stimulus is so applied, whereby it serves to activate the reaction that results in a change in the optical property. The change in the optical property of the reaction mixture (i.e., the dye disappearing) does not occur in the absence of the light stimulus; the activation provided by the light stimulus is correlated with a profound increase in the rate of change of the optical property of the reaction mixture.

The rate of change in the optical property of the mixture is preferably compared to a reference value characteristic of the rate of change of the optical property in a similar mixture containing a known amount (which can be a zero amount) of the NAA/TP hybrid to determine a relative rate of change in the optical property. The relative rate of change in the optical property of the mixture is correlated with the presence or amount of the target polynucleotide in a sample to determine the presence or amount of target polynucleotide in the sample.

An alternative method substitutes the comparison of rates of change to an observation of the optical property after exposure of the reaction mixture to a light stimulus. One can conclude that the target polynucleotide is present to the extent that an observeable change in the optical property occurs after the light stimulus exposure. One can also approximate the amount of the target polynucleotide that is present in the sample by comparing the optical property of the light-stimulus exposed reaction mixture after or upon a minimal or certain incubation time to a standard chart that displays the observable optical property as it will appear by the minimal or at the certain time. The standard chart is preferably generated by conducting the detection method with samples having known amounts of the target polynucleotide, memorializing the standard resultant state of optical property by, for example, taking color photographs of the mixtures after the minimal or at the certain time of incubation, and assembling the standard chart using the memorialized record of the state of the optical property of the mixture. Of course, one must take care that the memorialization as to quality (color) and/or quantity (intensity) is substantially accurate. Alternatively, the values of the standard chart can be generated by an equation.

A reference value can be a value characteristic of a property of a composition or compound having a known characteristic. For example, in various embodiments, a reference value can be determined using a mixture that does not contain an NAA/TP hybrid; contains a known amount of an NAA/TP hybrid; or is a reaction mixture from which one or more components (e.g., a nucleic acid analog, a target polynucleotide, or a dye) has been omitted, each of which are “controls” of the inventive method. In some instances, the reference value may be a known quantity or a measured quantity. Further nonlimiting examples of reference values include a value characteristic of an optical property of a mixture that has not been exposed to light stimulus, or, in an alternative embodiment, an optical property of a mixture that has been exposed to light stimulus. The aforementioned examples are for illustration and are not intended to limit the methods, and other examples will be apparent to the practitioner guided by this disclosure. It will be appreciated that a reference value may be, but need not necessarily be, empirically determined. For example, if it is known that the optical properties of a composition containing a dye do not change, or change minimally, in the absence of the NAA/TP hybrid, the reference value may be calculated or inferred and not measured. The reference value may be a constant. Although in some cases it may be convenient to assay a “control” sample concurrently with test samples, in more preferred embodiments of the method it is not necessary to do so. A reference value can be determined at one time point, and the value recorded for comparison at later time points as in the standard chart noted above. It will be understood that the aforementioned examples are for illustration and not limitation.

In one aspect, the reference value is characteristic of the rate of change in the optical property of a similar mixture containing no NAA/TP hybrid. In one embodiment, the reference value may be characterized by the optical property contributed by the dye prior to the combination of all the components in the mixture. Alternatively, the reference value can be external to the mixture, as in a color affixed to the vessel in which the reaction mixture is located. In another embodiment, the reference value may be characterized by the optical product of the reaction. For embodiments in which the mixture is exposed to light stimulus, the reference value may be characteristic of the optical property of the dye or mixture containing the dye prior to applying the light stimulus. It is not a requirement of the present invention that the reference value is necessarily determined by preparing one or more control samples that are included in separate mixtures and otherwise treated substantially identically as the experimental sample. In addition, it is understood that the reference value may be a constant.

The present application is further directed to methods in which (i) a sample containing, or not containing, or possibly containing a target polynucleotide, (ii) a nucleic acid analog that binds a target nucleic acid sequence of the target polynucleotide in a sequence-specific manner, and (iii) a dye are preferably combined to form a mixture. A light stimulus is preferably applied and the intensity of an optical property of the mixture is observed. In a preferred embodiment, the decrease in intensity of the mixture is correlated to the presence or amount of target polynucleotide in the sample. Alternative methods for observing the reaction include degtection of the product of the photochemical reaction or detection of a previously hidden, masked or quenched component.

In certain embodiments, a dye preferably exhibits an initial color change when in the presence of an NAA/TP hybrid. Following the initial color change, the color of the mixture decreases until the mixture becomes substantially clear (i.e., lacks or nearly lacks color). The rate of the change in the optical property corresponding to the change of the mixture from the presence of a color to the substantial lacking of color is preferably measured. The rate of change in the optical property can thereby be determined.

In still other methods, a sample and a non-PNA nucleic acid that is complementary to a target nucleic acid sequence in a target polynucleotide are combined with a dye to form a mixture. The mixture has a different optical property in the presence or absence of an NAA/TP hybrid. A change in the optical property of the mixture correlates to the presence of a target nucleic acid sequence.

In another embodiment of the invention, the time required to reach a specific change in an optical property is measured, and the percent change thereof is preferably calculated. Alternatively, the change in the optical property at a specific timepoint can also be measured, or merely observed where an approximate determination will suffice. For example, if the amount of target polynucleotide in a sample is known, the measured optical property at a specific time can be employed in assessing the relative amount of the target polynucleotide in a second sample of unknown target polypeptide content. Observing the optical property change at the specific time and comparing it to the characteristic of the known sample at the same point in the reaction the observer can conclude that the second sample contains a greater, about the same, or lesser or amount concentration of the target polypeptide as exists in the known sample. The amount or quantity of the target nucleic acid could thus be determined in a binary fashion, and the amount as well, albeit to an approximation or a comparative value of lesser or more.

A. Dyes

A group of related dyes are preferably used in the methods hereof to detect the presence or amount of a target polynucleotide.

In one embodiment, the preferred dye is a compound that is represented by the formula (I), or a salt or betaine thereof:

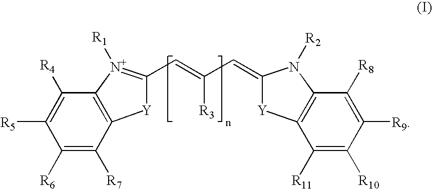

### In formula (I), independently at each occurrence,

R₁ and R₂ are each independently selected from hydrogen, alkyl, alkenyl, alkynyl, heteralkyl, heteroalkenyl, heteroalkynyl, aryl, acyl, heteroacyl, heteroaryl, hydroxyl, alkoxy, carbonyl, sulfinyl, sulfonyl, and amino groups;
R₃ is selected from the group consisting of hydrogen, alkyl, alkenyl, alkynyl, aryl, heteroalkyl, heteroalkenyl, heteroalkynyl, aryl, acyl, heteroacyl, heteroaryl, aryl, alkyl, heteroarylakyl, hydroxyl, alkoxy, halo, carbonyl, sulfinyl, sulfonyl, and amino groups;
R₄, R₅, R₆, R₇, R₈, R₉, R₁₀, R₁₁, R₁₂, and R₁₃ are each independently selected from the group consisting of hydrogen, halogen, alkyl, alkenyl, alkynyl, heteroalkyl, heteroalkenyl, heteroalkynyl, aryl, acyl, heteroacyl, heteroaryl, aryl, alkyl, heteroarylakyl, hydroxyl, alkoxy, carbonyl, sulfinyl, sulfonyl; wherein
n is 0 1, 2, 3, 4, or 5; and
Y is selected from the group consisting of —C₁₂═CR₁₃—, sulfur, nitrogen, and oxygen. A more preferred dye is the compound of formula (I), wherein Y is —C₁₂═CR₁₃—, sulfur, or oxygen. A yet more preferred dye is the compound of formula (I), wherein Y is sulfur; or wherein Y is C₁₂═CR₁₃—; or wherein Y is oxygen.

In another embodiment, the dye is preferably a compound that is represented by the formula (II), or a salt or ester thereof:

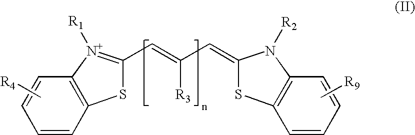

In formula (II), independently at each occurrence,
R₁ and R₂ are each independently selected from C₁-C₆ alkyl, C₂-C₆ alkenyl, and C₂-C₆ alkynyl;
R₃ is selected from the group consisting of hydrogen, C₁-C₆ alkyl, C₂-C₆ alkenyl, and C₂-C₆ alkynyl, C₆-C₁₀ aryl, hydroxyl, alkoxy, carbonyl, sulfinyl, sulfonyl, and amino groups;
n is 1 or 2;
R₄ and R₉ are each independently selected from the group consisting of H, C₁-C₆ alkyl, C₂-C₆ alkenyl, C₂-C₆ alkynyl, C₆-C₁₀ aryl, hydroxyl, alkoxy, halo, carbonyl, sulfinyl, sulfonyl, and amino groups.

In a further embodiment, the dye is a compound that is represented by the formula (III), or a salt or ester thereof:

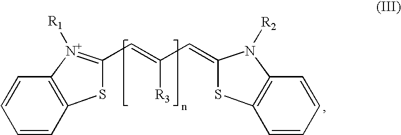

In formula (III), independently at each occurrence:
R₁ and R₂ are each independently selected from C₁-C₆ alkyl and C₂-C₆ alkenyl;
R₃ is selected from the group consisting of hydrogen and methyl;
n is 1 or 2.

In yet other embodiments, the dye is preferably selected from the following compounds:

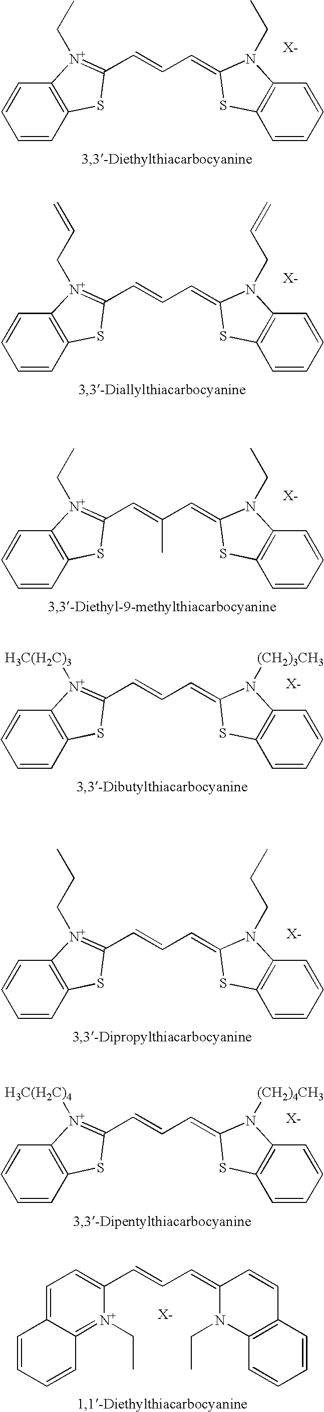

where X— is an anion. More preferably, the anion is a halogen; yet more preferably, the anion is iodide.

In one embodiment of formula (I, II, and III), n is 0.

In another embodiment of formula (I, II, and III), n is 1.

In another embodiment of formula (I, II, and III), n is 2.

In yet another embodiment of formula (I, II, and III), n is 3.

In one embodiment of formula (I, II, and III), R₁ and R₂ are each independently selected from group consisting of hydrogen, alkyl, alkenyl, alkynyl, heteralkyl, heteroalkenyl, heteroalkynyl, aryl, and heteroaryl.

In another embodiment of formula (I, II, and III), R₁ and R₂ are each independently selected from group consisting of alkyl, alkenyl, alkynyl, heteralkyl, heteroalkenyl, and heteroalkynyl.

In another embodiment of formula (I, II, and III), R₁ and R₂ are each independently selected from group consisting of alkyl, alkenyl, and alkynyl.

In another embodiment of formula (I, II, and III), R₁ and R₂ are each independently alkyl.

In one embodiment of formula (I, II, and III), R₃ is selected from the group consisting of hydrogen, alkyl, alkenyl, alkynyl, heteroalkyl, heteroalkenyl, andheteroalkynyl.

In one embodiment of formula (I, II, and III), R₃ is selected from the group consisting of alkyl, alkenyl, and alkynyl.

In one embodiment of formula (I, II, and III), R₃ is alkyl.

The following compounds are formally within the scope of formulas (I-III). Unfortunately, they did not perform under the conditions set forth herein for conducting a diagnostic test of the present invention. Preferably, therefore, dyes used in the context of the methods of the present invention are not selected from the following list: 3,3′-Diethylthiacyanine; 3-Ethyl-9-methyl-3′-(3-sulfatobutyl)thiacarbocyanine; 3,3′-Dimethyloxacarbocyanine; 3-Carboxymethyl-3′,9-diethyl-5,5′-dimethylthiacarbocyanine; 3,3′-Diethylthiadicarbocyanine; 3,3′-Diethylthiatricarbocyanine; 3,3′-Diethyloxacarbocyanine; 3,3′-Diethyloxadicarbocyanine; 3,3′-Dipropylthiadicarbocyanine; 3,3′-Dipropyloxacarbocyanine; 3,3′-Dihexyloxacarbocyanine; 3,3′-Diethyl-2,2′-oxathiacarbocyanine; 1,1′-Diethyl-2,2′-cyanine; 1,1′-Diethyl-2,4′-cyanine; 1,1′-Diethyl-4,4′-carbocyanine; 1,1′-Diethyl-3,3,3′,3′-tetramethylindocarbocyanine; 1,1′-Dipropyl-3,3,3′,3′-tetramethylindocarbocyanine; [5-[2-(3-Ethyl-3H-benzothiazol-2-ylidene)-ethylidene]-4-oxo-2-thioxo-thiazolidin-3-yl]-acetic acid; 1-Butyl-2-[3-(1-butyl-1H-benzo[cd]indol-2-ylidene)-propenyl]-benzo[cd]indolium; 5,6-Dichloro-2-[3-(5,6-dichloro-1,3-diethyl-1,3-dihydro-benzimidazol-2-ylidene)-propenyl]-1,3-diethyl-3H-benzimidazolium; 1,3,3-Trimethyl-2-(2-[2-phenylsulfanyl-3-[2-(1,3,3-trimethyl-1,3-dihydro-indol-2-ylidene)-ethylidene]-cyclohex-1-enyl]-vinyl)-3H-indolium; 4,5,4′,5′-Dibenzo-3,3′-diethyl-9-methyl-thiacarbocyanine; and Thiazole orange.

Suitable dyes can be identified using any of a variety of screening methods. The suitability of a dye is a function of its ability to contribute an optical property to the reaction mixture, as described here, which optical property changes in response to and preferably, to the extent of the presence of a hybrid between either (1) a nucleic acid analog (“NAA”) and a nucleic acid (“NA”), i.e., an NAA/NA; or (2) between two complementary NAs, i.e., a NA/NA. By way of example and not limitation, a NAA/NA, or a NA/NA, or an NAA/TP (collectively referred to as the “hybrid”) is prepared for identifying suitable dyes. Preferably, the combination includes other components and is maintained in conditions to promote formation of the hybrid, using such components and conditions that are well known in the art. The candidate dye is then preferably added; more preferably, the candidate dye is added to separate aliquots of the combination, such that the dye is present in the separate aliquots at varying concentrations. Yet more preferably, a detergent, or a detergent and an alcohol, is added. The order of addition is not critical; the components can be added in any order. Once the reaction mixture is formed, a light stimulus is preferably applied, although not necessarily. The rate of change in the optical property over time is then determined. This rate is compared to a reference value characteristic of the rate of change in optical property of the reaction mixture in the absence of the hybrid. Example 4 illustrates one method for screening dyes for suitability for the inventive methods disclosed herein.

In certain embodiments, the reference value is characteristic of the absence of the target polynucleotide or the presence of the target polynucleotide, which can be single-stranded or double-stranded. In other embodiments, the reference value is characteristic of a non-zero concentration of the target polynucleotide. Preferably, the reference values employed include those characteristic of a zero and at least one non-zero concentration of the target polynucleotide, respectively.

In certain embodiments, the change in optical property can be the loss of intensity of the optical property. Dyes that contribute an optical property to the reaction mixture are particularly preferred, where the mixture exhibits a different rate of change in optical property over time compared to a reference value. The relative rate of change in the optical property of the mixture is correlated with the presence or amount of the target polynucleotide.

Alternatively, dyes that contribute an optical property to the reaction mixture, where the reaction mixture changes color in the presence of an NAA/NA hybrid, are preferably selected. Preferably, the nucleic acid analog used in the context of a color changing optical property is a non-PNA variety. Such nucleic acid analogs include, without limitation, an LNA, TNA, MLNA, and a morpholino nucleic acid.

B. Designing Nucleic Acid Analog Sequences

For use in the present invention, nucleic acid analogs may be designed to be complementary, but possibly including some mismatched bases, or exactly complementary to a nucleic acid analog.

The sequence of the nucleic acid analogs or target polynucleotides may be designed in a variety of ways. By way of example and not limitation, the nucleic acid analogs or their respective complementary polynucleotides may be designed to have sequences based on known primers used for PCR-based amplification and detection of specific target sequences. The nucleic acid analog may also be designed to be complementary or exactly complementary to any target nucleic acid sequence of the target polynucleotide. Alternatively, the nucleic acid analog may be designed to have a one base mismatch or a two base mismatch. In certain embodiments, the sequence of the nucleic acid analog may be based on the sequence of PCR primers used to detect polynucleotides associated with pathogens, the presence of a pathogen in a host, a disease gene, a genetic condition, or a genetic change associated with a physiological change or condition. The nucleic acid analog may also be complementary or exactly complementary to all or part of the sequence encoding the active or functional domains of a protein and/or the intact protein and or non-coding sequences (e.g., regulatory sequences, introns etc).

One of skill in the art, guided by this disclosure, will recognize that in addition to nucleic acid analogs specifically listed herein, other nucleic acid analogs (including nucleic acid analogs discovered or developed in the future) may be used in the methods of invention. Nucleic acid analogs that form an NAA/TP hybrid under the assay conditions described herein are suitable for the present methods, and affect the rate of change in an optical property.

Exemplary nucleic acid sequences that can be used in the methods disclosed herein include but are not limited to those listed in Table 1. These and other sequences can be used with respect to target polynucleotides or nucleic acid analogs, and can be modified to include non-nucleic acids located at either terminus of the nucleic acid analog or anyplace in between the termini. Useful modifications include the addition of any natural or non-natural amino acid residues or a protein; amino acids may be present as single residues or present as a polypeptide; preferred amino acid residues include lysine and glycine. Preferred proteins that can be attached to such nucleic acid sequences include those that specifically bind a ligand; more preferred proteins include an antibody (specific for an antigen) and a ligand (specific for a sugar); a yet more preferred protein is biotin (specific for avidin or strepavidin). Other sequences include complementary sequences of those listed in Table 1.

TABLE 1

Exemplary targets for designing nucleic acid
analog probes.

| | | |
|---|---|---|
| Target | Sequence (5′ to 3′) | SEQ ID NO: |

| | | |
|---|---|---|
| 35S CMV promotor | GATAGTGGGATTGTGCGT | 1 |
| Maize zein control | ACAGTTGCTGCA | 2 |
| Maize invertase | TGTATCACAAGG | 3 |
| Maize adh | CTCCGAGACCCT | 4 |
| Soy lectin | CTATTGTGACCT | 5 |
| Shiga-like toxin 1 | TCGTTGACTACT | 6 |
| Shiga-like toxin 2 | AACTGCTCCTGT | 7 |

Nucleic acid analogs can hybridize rapidly to target polynucleotides. PNA hybridization to polynucleotides, for example, is independent of salt concentration. See Demidov et al., BIOCHEM. PHARMACOL. 48:1310-3 (1994). PNAs are resistant to nuclease and protease attack, and bind to polynucleotides more specifically than conventional DNA probes. Short probes can be used with great sequence specificity (Ray and Norden, FASEB J. 14:1041-60 (2000)). Furthermore, PNA/polynucleotide hybrids have higher thermal stability than the corresponding DNA/polynucleotide hybrids, and the melting point of PNA/polynucleotide hybrids is relatively insensitive to ionic strength, showing equal thermal stability under low (<10 mM NaCl) and moderate (500 mM NaCl) salt concentrations. This ability of PNA/polynucleotide hybrids to form under low salt conditions is significant because the internal structure of dsRNA and rRNA is significantly destabilized at salt concentrations below 200 mM. Therefore, assay conditions can be chosen that favor the disruption of the target nucleic acid while still promoting formation of PNA: DNA hybrid molecules (Stefano and Hyldig-Nielsen, Diagnostic Applications of PNA Oligomers, in DIAGNOSTIC GENE DETECTION AND QUANTIFICATON TECHNOLOGIES 19-39 (Minden ed., 1997). PNA/polynucleotide hybridization is severely affected by base mismatches and PNA molecules can maintain sequence discrimination up to the level of a single mismatch.

ncPNA molecules may be purchased, for example, from Eurogentec (UK), Bio-Synthesis Inc. (Lewisville, Tex.), and Applied Biosystems Inc. (ABI), or synthesized by methods known in the art. In certain embodiments, the nucleic acid analogs can be a chiral PNA. ncPNA molecules can by synthesized, for example, according to the methods described in Mayfield and Corey ANAL BIOCHEM. 268(2):401-4 (1999), or Braasch et al., CURRENT PROTOCOLS IN NUCLEIC ACID CHEMISTRY. Unit 4.11 Synthesis and Purification of Peptide Nucleic Acids, pp. 14.11.11-14.11.18. John Wiley & Sons, New York. Chiral PNAs can be synthesized, for example, according to the methods disclosed by Kumar et al., ORG LETT. 3(9):1269-72 (2001) or D'Costa et al., ORG LETT. 1(10):1513-6 (1999).

In other embodiments, the preferred nucleic acid analog includes one or more LNAs. LNAs may be purchased, for example, from Proligo (Boulder, Colo.). In still other embodiments, the nucleic acid analog is preferably a morpholino nucleic acid analog or a TNA. TNAs can be synthesized, for example, according to the methods disclosed by Chaput and Szostak, J. AM. CHEM. SOC. 125(31):9274-5 (2003). Morpholino nucleic acids can be purchased, for example, from Gene Tools (Philomath, Oreg.). A comparison between chiral PNA, LNA, morpholino nucleic acid analogs and non-chiral PNA analogs is disclosed in Example 3. The nucleic acid analogs produced reduced fluorescence intensities of the dye at various levels when compared to the negative control reaction containing nucleic acid analog with dye.

C. Target Polynucleotides

The target polynucleotide may be any polynucleotide, including naturally occurring, synthetic, and amplified polynucleotides. Other types of polynucleotides may be single-stranded, double-stranded, triple-stranded, or yet greater degree multi-stranded. Non-limiting examples of target polynucleotides include DNA, RNA, regulatory RNA, mRNA, regulatory microRNA, siRNA, artificial RNA, chimeric RNA, and armored RNA. Other non-limiting examples of target polynucleotides include epigenomic DNA, epigenetic DNA, in vitro amplified DNA, and chimeric DNA. The target polynucleotide may contain single nucleotide polymorphisms (SNPs) that are identified or quantitated by the methods disclosed herein.

D. Detergents

Any detergent can be usefully included with the reagents individually or with the reaction mixture. Indeed, the reaction mixture preferably includes detergent. The advantages of adding detergent to the reaction mixture were observed and are reported in Example 1 hereof. As can be seen in detail, the addition of detergent results in greater relative signal for test reaction mixtures as compared to negative control reaction mixtures. Furthermore, the addition of detergent reduces photobleaching of samples containing only dye and/or only dye and nucleic acid analog.

The detergent used in the context of the present invention, then, can be a cationic detergent, anionic detergent, nonionic detergent, or zwitterionic detergent. Non-limiting examples of cationic detergents include, for example, tetramethyl ammonium chloride (TMAC) (Sigma, St. Louis Mo.). Non-limiting examples of anionic detergents include N-lauroyl sarcosine sodium salt (LSS) and sodium dodecyl sulfate (SDS) (Sigma-Aldrich, St. Louis Mo.). Non-limiting examples of nonionic detergents include Tween® 20, Tween® 40, Tween® 80, NP40 (Tergitol®), Triton® X-100, Span® 20, and Span® 80 (Sigma, St. Louis Mo.). Non-limiting examples of zwitterionic detergents include 3-[(3-Cholamidopropyl)dimethylammonio]-1-propanesulfonate (CHAPS) (Sigma, St. Louis Mo.). The detergent is preferably included in the mixture itself and/or in the composition that includes the dye. Suitable concentration for the detergent ranges from about 0.01% to about 2%; more preferably from about 0.05% to about 1%; yet more preferably, about 0.05%, about 0.01%, about 0.5%, or about 1%.

E. Additional Additives

Other compounds that can be added to the reaction mixture include, but are not limited to, 1,4-diazabicyclo[2,2,2]octane (DABCO), p-phenylenediamine (PPD), n-propyl gallate (NPG), ascorbic acid, sodium azide, polyvinyl pyrrolidone (PVP), and glycerol.

The reaction mixture can also incorporate other compounds and reagents. Examples of preferred other compounds or reagents include phosphate buffers, water, and alcohol. Suitable alcohols include butanol, methanol, and isopropanol. Alcohol is preferably included in a range of concentration of about 1% to about 15% on a v/v basis; more preferably, about 3%, about 5%, about 10%, or about 14% alcohol. DMSO can also be included, at concentrations of about 1% to about 12%; more preferably at about 10%.

F. Buffer Systems

Suitable buffers for the reaction mixture include: 50 mM KCl, 10 mM Tris HCl, and 0.1% Tritone X-100; 0.5% Tweeno 20 in 5 mM phosphate buffer, pH 5.5; 0.5% Tween® 40 with 5 mM phosphate buffer, pH 5.5; 0.5% NP40 with 5 mM phosphate buffer, pH 5.5; 0.05% lauryl sarcosine with 5 mM phosphate buffer, pH 5.5; 5 mM phosphate buffer, pH 5.5, with 0.05% Tweene 80 and 14% methanol; blood lysis buffer (0.15 M NH₄Cl, 10 mM NaHCO₃, 0.1 mM EDTA pH 7.4); and sucrose lysis buffer (0.32 M sucrose, 10 mM Tris, 1% Triton® X-100, 5 mM MgCl₂). Various concentrations, pH, and combinations of the aforementioned compounds and reagents as well, but not limited to the above, can be usefully employed with the present invention. Target polynucleotides can be detected in samples that contain cells or tissues. The addition of a detergent preferably permeabilizes and/or lyses cells without requiring purification or separation of polynucleotides from other components of a sample. The target polynucleotide is preferably detected directly in the permeabilized cells or crude cell lysate; the target polynucleotide does not need to be additionally purified or isolated from the mixture.

The addition of alcohol to a reaction mixture containing the detergent further reduces the photobleaching of the dye in the absence of an NAA/NA hybrid, but does not reduce the change in absorbance or fluorescence of the reaction mixture when the NAA/NA hybrid is present. This aspect of the present invention is surprising because in the absence of detergent, the addition of alcohol slows the change in optical property. In preferred embodiments, about 8-12% ethanol or about 12-15% methanol is added in tandem with a detergent.

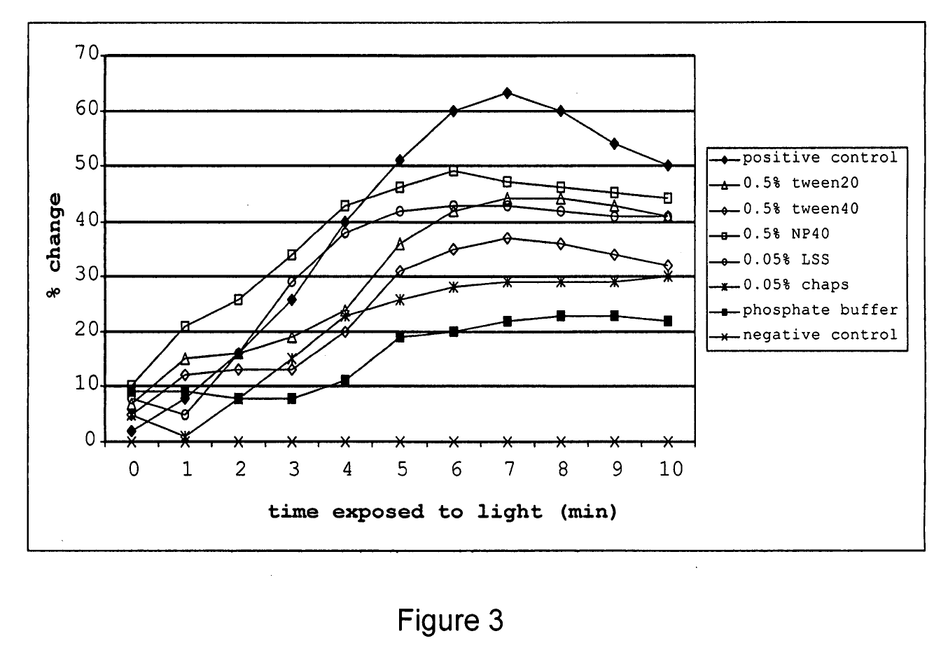

An example showing the detection of a target polynucleotide from a detergent-treated bacterial sample will assist in describing the usefulness of both the method and the inclusion of surfactant. With reference to Example 2 and FIG. 3, individual buffer solutions, containing various detergents, were used to permeabilize and/or lyse bacterial cells. After 10 minutes incubation, 5 μl from detergent-treated bacterial cells were transferred to a buffer containing achiral PNA (a nucleic acid analog) and 3,3′-diethylthiacarbocyanine iodide dye, thus forming the test reaction mixture. Fluorescence intensity (535 nm excitation, 590 nm emission) of reactions was read at time zero. Reactions were exposed to light for one minute and re-read. This exposure-reading cycle was repeated to 10 minutes total exposure. For each detergent, the “percent change” in fluorescence intensity at each timepoint was calculated as per the formula: 100−[RFU_{TW}−RFU_{NC}]×100, where RFU_{TW} is the measured fluorescence intensity of the test reaction mixture, and RFU_{NC} is the measured fluorescence intensity of the negative control mixture (i.e., ncPNA, no target nucleic acid, dye, in buffer containing each type of detergent). Magnitude of percent changes corresponds to relative signal intensity (test reaction relative to negative control reaction mixtures) in the detection of the presence of the target nucleic acid sequence. A consistent positive percent change indicates a consistently stronger signal from the test reaction mixture as compared to the negative control mixture, indicating a consistent ability for the system to detect TP. Detergents increase the relative signal, allowing the easier detection of TP in a detergent-treated sample. The effect of some detergents is more dramatic than that of others. Notably, the presence of a target nucleic acid sequence can be determined from the detergent-treated cells, without requiring further purification of the target polynucleotide.

The presence or quantity of a target nucleic acid in the target polynucleotide may be detected in any group of cells or tissues. For example, the presence of a target nucleic acid can be detected in a tissue culture, cells, tissues or fluids obtained from an animal or plant. In other embodiments, the presence or absence of a target polynucleotide in any tissue or fluid can be determined. Non-limiting examples of such tissues or fluid include urine, blood, saliva, lacrimal fluid, inflammatory exudates, synovial fluid, abscess, emphysema or other infected fluid, cerebrospinal fluid, sweat, pulmonary secretions (sputum), seminal fluid, feces, and plant tissue. Cancer cells or other cells circulating in blood can be detected, or cells having different RNA expression levels. Other examples include detection of cell (animal or bacterial) transformation, or the absence of contamination in tissue cultures.

G. Light Stimulus

Light stimulus can be provided to a sample, nucleic acid analog, and dye mixture either concurrently with the production of the mixture or at a specified time after the production of the mixture. The light stimulus causes a different rate of change in an optical property of the mixture.

The light stimulus may be in the visible spectrum or outside the visible spectrum. The light stimulus may be white light of a number of wavelengths. Alternatively, the light stimulus may be a specific wavelength or wavelengths, or range of wavelengths.

Light sources are known in the art. Different light sources result in different reaction rates because of differences in intensity or wavelength of the light sources. Examples of light sources include Xenon arc lamp (Ushio, #UXL-451-O), Sylvania dulux S9W CF9DS/blue and Sylvania Cool White T8-CW (OSRAM SYLVANIA, Danvers, Mass.), General Electric T8-C50 GE Lighting, Cleveland, Ohio), Osram F9TT/50K (OSRAM GmbH, Munich, Germany), and Fritz Aurora 50/50 (Fritz Industries, Inc., Mesquite, Tex.). Other light sources include light emitting diodes (LEDs) that produce a specific range of wavelengths. LEDs emit light at at least one peak wavelength, and in certain embodiments can emit light at multiple peaks. In certain variations, the bandwidth of the LED can be as small as 1 nm, or as large as 20 nm. Other light sources include commercially available halogen light sources, such as halogen headlamps (NAPA Auto Parts, Atlanta, Ga.).

The light stimulus may also have a specific intensity. In certain variations, a 15-Watt light source at 555 nm produces between about 400 foot-candles and 2000 foot-candles of illumination. In other variations, the light stimulus is one or more LEDs, preferably it is a bank of LEDs, the power of which varies from 500 μW to 4000 μW/cm² at 3.5 inches away from the light.

Those of skill in the art will recognize that the optimal light stimulus may be determined without undue experimentation for a specific dye, or a specific nucleic acid analog, polynucleotide, and dye mixture. A single set of temperature and concentration conditions can be optimized for a specific mixture. A source of the light stimulus can also be optimized for illuminating a plurality of hybridizations.

H. Forming Alternative Target Polynucleotide/Nucleic Acid Analog Hybrids

Assays for detection of target polynucleotides can be carried out using a variety of hybridization schemes. In one format, the polynucleotide sequence may be identified by hybridization of a target polynucleotide directly to a nucleic acid analog to form a target polynucleotide/nucleic acid analog hybrid, which is described above. Here, we will present two additional schemes for detecting the target polynucleotide by means of a change in the optical property of the reaction mixture, as follows:

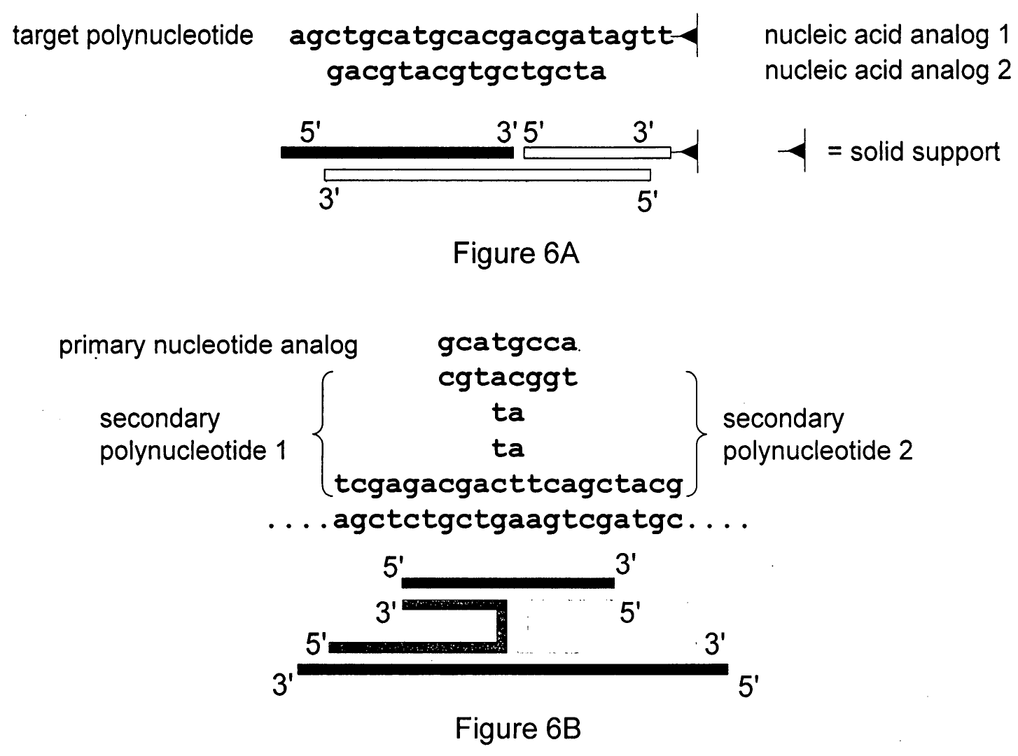

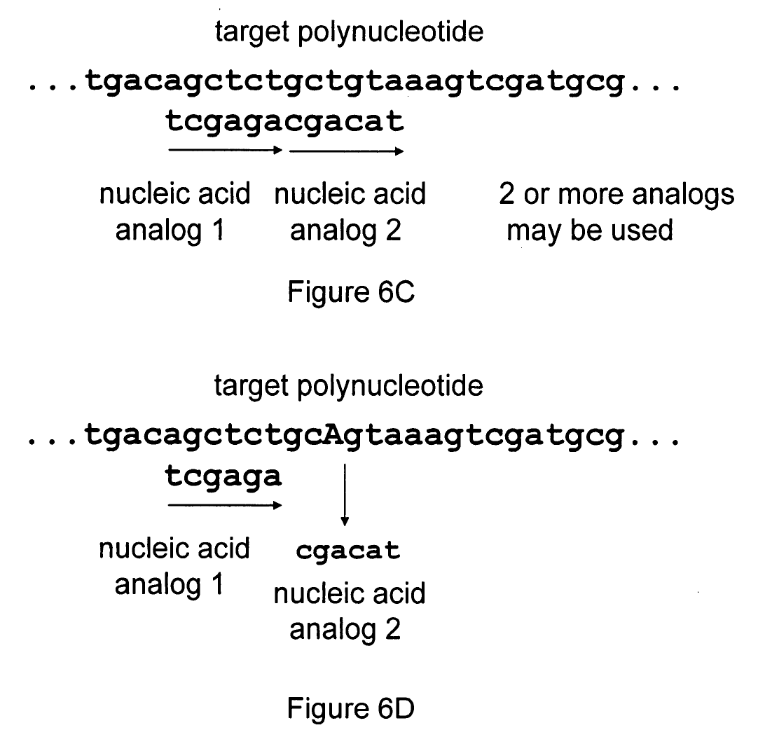

In one aspect, and as depicted in FIG. 6A, a nucleic acid analog 2 molecule hybridizes to a portion of a complementary target nucleic acid target sequence. The nucleic acid analog can then hybridize to a second nucleic acid analog 1 molecule that is preferably immobilized on a solid substrate, as depicted in FIG. 6A. This may be accomplished in a one-step or multistep process. In a one step process, the target polynucleotide and nucleic acid analogs are combined simultaneously. In a multistep process, the target polynucleotide and nucleic acid analogs are combined sequentially.

In another aspect, the presence of a target polynucleotide may be detected by forming a branched reaction crucifix form structure, an example of which is depicted in FIG. 6B. In this format, a target polynucleotide is hybridized to two secondary polynucleotides 1 and 2 that are complementary to non-overlapping and contiguous portions of the target polynucleotide. The secondary polynucleotides form a branched structure that also hybridizes to a primary nucleic acid analog. The target hybridizing regions of the intermediate polynucleotides may be designed to be too short for a dye to bind the primary nucleic acid analog separately, but large enough to bind the primary nucleic acid analogs or target polynucleotides when hybridized.

Without relying on any particular mechanism of action, the change of color or fluorescence of a dye has been found to depend on the length of the nucleic acid analog. The optical property contributed by the dye changes if the nucleic acid analog is at least about 10 bases long. Accordingly, by employing a sufficient short primary nucleic acid analog in the mixture, the rate of optical change of the reaction mixture may then be determined after the step of allowing hybridation to occur. In the absence of the hybridization of the primary nucleic acid analog to the secondary polynucleotides, no substantial change in the optical property of the reaction mixture is observed. Unless the secondary polynucleotides hydridize to the target polynucleotide and to the primary nucleic acid analog, the optical property of the reaction mixture remains substantially stable.

In a preferred embodiment, the single nucleic acid analog is a universal nucleic acid analog that is used for all assays and optimized for effective changes in the optical property of a dye. The universal nucleic acid analog could be used for any target nucleic acid and the secondary sequences could be varied to be specific for a given target polynucleotide. This scheme can be adapted to a format using an immobilized nucleic acid analog. This universal sequence can be optimized for a given set of reaction conditions.

In another format, multiple nucleic acid analogs or target polynucleotides form an NAA/TP hybrid with adjacent regions of a target polynucleotide. In this format, each nucleic acid analog is preferably too short for the reaction mixture's optical property to change, although it does change at the background rate; but multiple nucleic acid analogs that are complementary to contiguous segments of the target polynucleotide preferably provide a large enough region for a rate of change in the optical property to result. As depicted in FIG. 6C, a target polynucleotide as a single molecule may form an NAA/TP hybrid with two or more separate nucleic acid analogs that hybridize to adjacent sequences. SNPs can be identified using the two separate nucleic acid molecules when there is a single base mismatch between the target nucleic acid and one of the SNPs. If all the bases in one nucleic acid analog cannot hybridize as in FIG. 6D then a change in optical property may not be observed.

I. Hybridization Conditions

The methods disclosed herein detect SNPs at room temperature. As a result, stringent hybridization conditions do not need to be used. Although hybridization conditions can be modified or optimized, generally the hybridization conditions within a reasonable range do not affect the ability to detect a target polynucleotide according to the methods disclosed herein.

If hybridization controls are changed or optimized, the design and/or choice of hybridization conditions is governed by several parameters. These parameters include, but are not limited to, the degree of complementarity of the nucleic acid analog to the target polynucleotide, the length of the nucleic acid analog to be utilized and the target polynucleotide itself. Preferred hybridization conditions allow for one or more of the following: efficient hybridization of nucleic acid analogs to target polynucleotides, minimization of RNA or DNA secondary structure, minimization of RNA degradation and either discrimination of one or more base pair changes or inclusion of one or more base pair changes.

Hybridization reactions can be performed under conditions of different “stringency.” Conditions that effect stringency of a hybridization reaction are widely known and published in the art. See, e.g., Sambrook et al. (2000), supra. Examples of relevant conditions include but are not limited to, salt concentrations, pH (buffers), and temperature. Hybridization conditions utilizing lower salt concentrations generally enhance DNA instability and PNA/polynucleotide stability. Examples of buffers that may be used include, but are not limited to, Na₃PO₄, NaHSO₄, K₂HPO₄, K₂SO₄, or CaSO₄. By way of example, the molarity of the buffers may range between about 10 mM and about 0.5 M and have a pH between about 4 to about 10, or between about 7 to about 10, such as about 7.0 or about 7.5. By way of example, Na₃PO₄ may be used at between about 0.5 mM and about 0.5 M, such as for example, 2.5 mM, and at a pH between about 4 to about 10 or between about 7 to about 10, such as about 7 or about 7.5.

Other buffer conditions include a 5 mM phosphate buffer, pH, 5.5 with 0.05% NP-40 (Tergitol®). Alternatively, the 0.05% NP-40 (Tergitol®) can be substituted with 0.05% Tween 80, optionally including about 10-14% methanol or about 8-12% ethanol. Examples of sample conditions include but are not limited to (in order of increasing stringency): incubation temperatures of about 25° C., about 37° C., about 50° C. and about 68° C.; buffer concentrations of 10×SSC, 6×SSC, 4×SSC, 1×SSC, 0.1×SSC (where SSC is 0.15 M NaCl and 15 mM of any buffer as described herein) and their equivalents using other buffer systems; formamide concentrations of 0%, 25%, 50%, and 75%; incubation times from about 5 minutes to about 24 hours; one, two, or more washing steps; wash incubation times of one, two, or 15 minutes; and wash solutions of 6×SSC, 1×SSC, 0.1×SSC, or deionized water. In one embodiment, hybridization and wash conditions are done at high stringency. By way of example, hybridization may be performed at high stringency using 50% formamide and 4×SSC followed by washes of 2×SSC/formamide at 50° C. and with 1×SSC.

Buffers may contain ions or other compounds, or different buffering capacity. Alternatively a component in the buffer may have a stabilization capacity; such as neomycin or other aminoglycosides, that stabilizes triplex DNA, (Arya et al; 2003) or naphthalene diamides that enhance triplex stability (Gianolio and McLaughlin, BIOORG. MED. CHEM. 9:2329-34 (2001)), or naphthylquinoline □yanoge (Keppler et al., FEBS LETT. 447:223-6 (1999)).

J. Mutation Detection

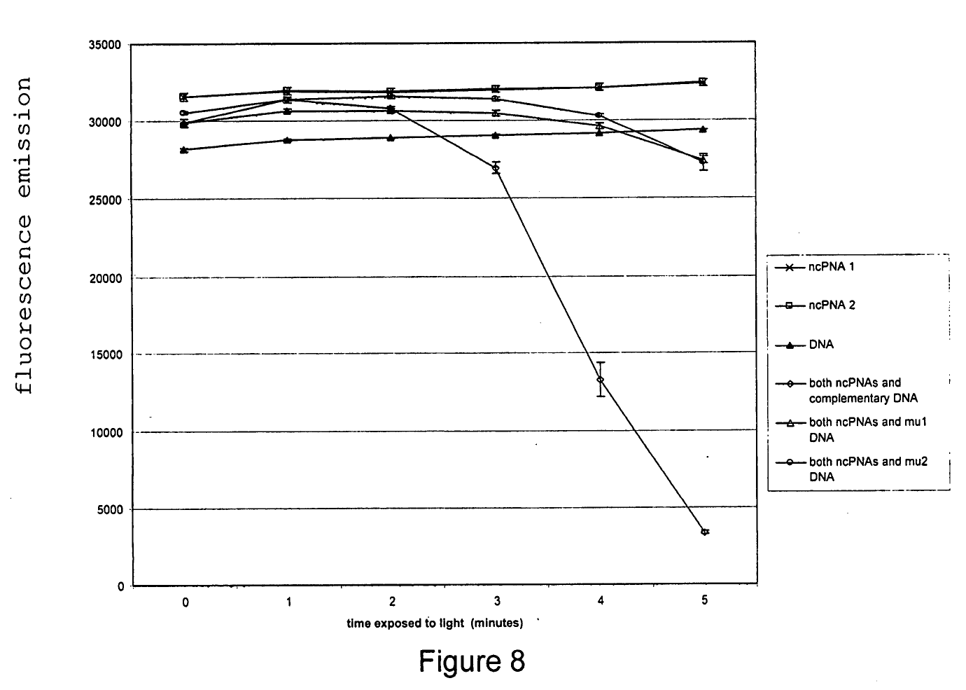

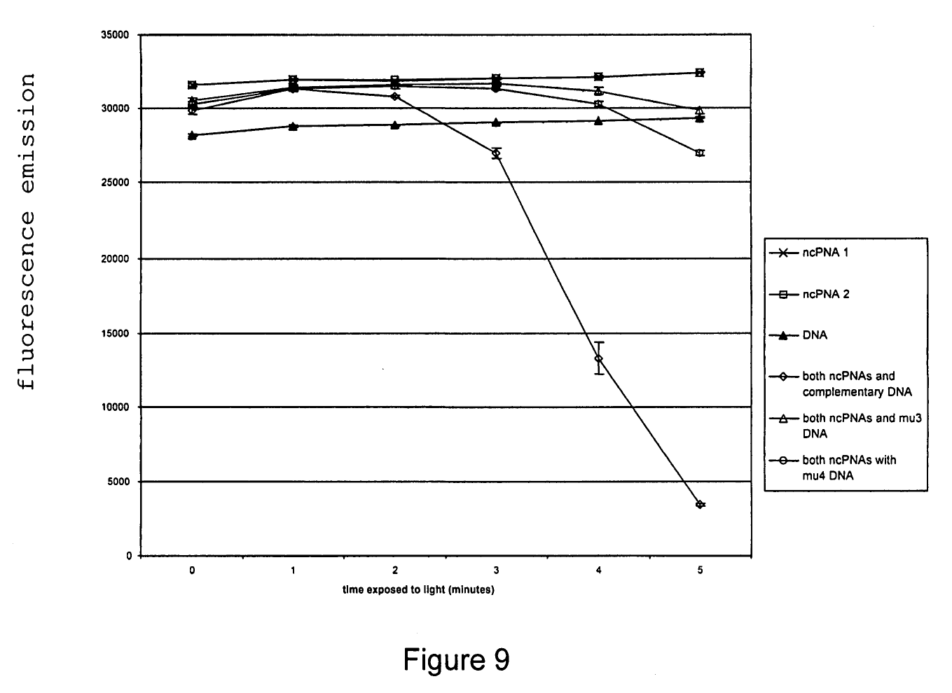

In one embodiment, nucleic acid analogs can be used to distinguish between polynucleotides having an exactly complementary sequence and one with a single base mismatch. For example and without limitation, nucleic acid analogs for use in the application may be designed to detect single nucleotide polymorphisms (SNPs). Formation of the NAA/TP hybrid is affected by base mismatches. According to the methods of the application, upon the addition of a dye, a single base mismatch between a target sequence (e.g., SNP) and a nucleic acid analog results in a slower rate of change in optical property of the mixture compared to a nucleic acid analog that does not have the mismatch. Example 7 depicts detection of a series of mutant SNPs. Single base mismatches along a double-stranded DNA were detected. FIG. 8 depicts the detection of SNPs DNA molecules with single base-pair changes. FIG. 9 depicts detection of DNA molecules with a two or a four base-pair change. The identification of SNPs for diagnosis and other methods is well known in the art.

In another embodiment, the nucleic acid analog may be designed to detect the presence or amount of a class of organisms. By class of organisms, it is meant that all organisms have one or more sequences that are complementary to, or exactly complementary to, a nucleic acid analog sequence. Such classes of organisms can be distinguished from other organisms based on the complementarity to nucleic acid sequences.

In yet another embodiment, the nucleic acid analog has a purine content of less than about 60%, and has a maximum of 4 purine bases or three guanine bases in a row. Purine-rich nucleic acid analogs tend to aggregate and have low solubility in aqueous solutions. The nucleic acid analogs are preferably selected to minimize or avoid self-complementary sequences with inverse repeats, hairpins and palindromes because these types of structures are prone to aggregate.

Nucleic acid analogs may hybridize to target polynucleotides in either orientation, but an anti-parallel orientation is preferred. Anti-parallel is the preferred configuration for antisense and DNA hybridization-type applications. When the orientation of the nucleic acid analog is anti-parallel, the N-terminal of the nucleic acid analog is equivalent to the 5′-end of the DNA. Both N′ and 5′ are used herein.

K. Automated Devices

Certain automated devices that can activate the reactions, record the results and/or include the software to assist in interpretation of the results are also contemplated within this invention. The device is preferably fully automated and perform all reaction steps after sample addition. The device can be a handheld unit, a mobile unit or a stationary lab unit.

In certain aspects, the device preferably detects changes in fluorescence. Fluorescence detection devices can have a light source for activating the dye and a light source for exciting the dye. The device determines the emission at a range of wavelengths. Alternatively, if the device detects absorbance, a first light source can activate the dye and a second light source can be used for absorbance measurements; alternatively, the instrument can use a single light source coupled with appropriate light filters. Possible test formats include but are not limited to, a test strip (e.g., nitrocelluose nylon), beads (e.g., latex or polystyrene), capillary tubes, monofilament, or in micro wells on an etched surface or plastic form.

Once a sample is exposed to an activating light source and read, the data can be gathered and stored in the device. The device preferably automatically tracks and calculates the change in fluorescence and produces a read out indicating whether a sample does or does not contain the target polynucleotide, and/or determine the amount of the target polynucleotide. The testing format can, for example, be bar coded to provide the unit with predetermined information on the test that may be used to increase the accuracy of the calculations.

### IV. Method Formats

The methods can be adapted to several different formats. Preferred formats include, without limitation, liquid-based formats, solid-substrate-based formats, and gel-based formats. It will be understood that various aspects of the formats can be combined.

A. Liquid-Based Formats

In a preferred embodiment, the method for detecting a target polynucleotide is liquid-based. The sample containing the target polynucleotide, a nucleic acid analog that binds a target nucleic acid sequence of the polynucleotide in a sequence specific manner, and the dye are preferably combined to produce a mixture in liquid solution. The misture preferably has an optical property that can change over time. The rate of change in the optical property of the mixture is compared to a reference value characteristic of the rate of change in the optical property of a similar mixture containing a known amount of an NAA/TP hybrid and dye to determine a relative rate of change in the optical property. The relative rate of change in the optical property of the mixture correlates to the presence or amount of the specified polynucleotide in a sample, thereby determining the presence or amount of polynucleotide in the sample. The change in the optical property of the mixture containing a nucleic acid analog or, upon adding a further step for introducing a light stimulus, a decrease in intensity of an optical property can be observed in a liquid-based format. The various methods disclosed herein may be performed in a liquid-based format.

The methods may also be conducted in any vessel, such as microfuge tubes, test tubes, and chips that hold a liquid by surface tension. The methods may also be conducted in multiwell plates. The plates may contain any number of wells. In one format, 96-well plates are used. In another format, 384-well plates are used. Any type of plate having any number of wells can be used. The wells themselves may be subdivided into smaller wells. When the assay format is in a microwell format, the liquid is retained in each well of a microtiter plate. In the alternative, or in addition, a gel matrix could be added to one or more wells.

B. Solid Substrate-Immobilized Formats

The nucleic acid analog or target polynucleotide may be immobilized on a solid substrate. The NAA/TP hybrid may be immobilized on a surface. The immobilization may be accomplished by any suitable method known in the art, the suitability of which is determined functionally, i.e., that immobilization act occurs without substantial detriment to the activity of the same compound when free in solution. In a preferred embodiment, either the nucleic acid analog or target polynucleotide is covalently linked to a second molecule that preferentially binds to a third molecule on the solid substrate.

One or more components are preferably immobilized on a solid substrate. For example, either the nucleic acid analog or the target polynucleotide may be immobilized. The NAA/TP hybrid may be immobilized either before or after formation of the hybrid. The dye may be added before or after immobilization and/or before or after hybridization. In other embodiments, the dye may be immobilized, in which case the situs of the mixture's optical change can be localized. Further, the light stimulus may be added either before or after immobilization on a solid substrate.

There are many types of solid substrates that the nucleic acid analog or target polynucleotide molecule may be attached to, including, but not limited to: cast membranes (e.g., nitrocellulose, nylon), ceramic, track-etched membranes (TEM), polyvinylidenedifloride, latex, paramagnetic beads, plastic supports of all types, glass, powdered silica or alumina on a support matrix or treated or untreated filter paper (e.g., Whatman FTA cards). If a grid pattern is used, the immobilized nucleic acid analog or target polynucleotide forms a microarray. In another preferred variation, the nucleic acid analog or target polynucleotide molecules are covalently modified to include a linking moiety, such as a biotin or amide linkage, which binds to membranes. In a further preferred variation, the nucleic acid analog or target polynucleotide molecules are immobilized via sequence-specific hybridization to one or more sequences. Any means of attaching a nucleic acid analog or target polynucleotide to a support is contemplated by the present application. In one aspect, the nucleic acid analog or target polynucleotide may be attached directly to a membrane. The nucleic acid analog may be a PNA (e.g., Giger et al., NUCLEOTIDES AND NUCLEOSIDE 17:1717 (1998)). A solution of nucleic acid analogs or target polynucleotides (in water) is preferably applied to a charged or chemically-modified filter and allowed to air dry. The filter is then used for hybridization.

In another embodiment, a biotin-labeled nucleic acid analog or target polynucleotide is attached to a streptavidin-coated surface, such as a bead or well (see, e.g., Chandler et al., ANAL. BIOCHEM. 283:241-249 (2000)). Biotin-labeled nucleic acid analog or target polynucleotide can be mixed with streptavidin-labeled latex or polycarbonate beads. The biotin binds strongly with streptavidin, allowing the nucleic acid analog or target polynucleotide to bind to the bead in a unidirectional fashion. The beads are then preferably applied to a non-charged membrane with a mesh size that is 25-30% greater than the diameter of the bead. Beads become trapped in the mesh, hence making a localized area of attached nucleic acid analogs or target polynucleotides. Direct synthesis of nucleic acid analog or target polynucleotides on a solid substrate, such as a polypropylene membrane, may be accomplished using standard 9-fluorenylmethoxycarboyl (Fmoc) protein synthesis chemistry (see, e.g., S. Matysiak et al., BIOTECHNIQUES 31:896-904 (2001)).

In another embodiment, the nucleic acid analogs or target polynucleotides are fixed to a glass or other solid substrate by applying a solution containing nucleic acid analogs or target polynucleotides in water directly to the glass or other support and letting it air dry.

In yet another embodiment, a nucleic acid analog is designed to produce a net positive charge, and may bind a negatively charged membrane. For example, a positively-charged lysine or glycine at a 5′ or 3′ end of the nucleic acid analog molecule may be used to attach the nucleic acid analog molecule to a negatively-changed nylon membrane. The negatively charged membrane repels any nucleic acid that is not complementary and/or exactly complementary to the nucleic acid analog, thus minimizing non-specific binding.

In yet another embodiment, nucleic acid analogs or target polynucleotides are preferably attached to microspheres as described by Xu et al., NUC. ACIDS RES. 31:e43 (2003). For each conjugation reaction, approximately 4×106 carboxylated microspheres (Polysciences, Warrington, Pa.) can be pelleted and washed with 0.1 M imidazole buffer pH 7.0. After resuspension of microspheres in 20 μl of imidazole buffer pH 7.0 (10% solids), 1 μl of 100 mM oligos, and 100 μl of 200 mM EDAC (Acros, Pittsburgh, Pa.) in freshly made imidazole buffer pH 7.0 is added and the reaction mixture is incubated for about 2 hours at room temperature with continuous rotation. An additional 100 μl of 200 mM EDAC in freshly made imidazole buffer pH 7.0 is added and the room temperature incubation with rotation was continued for another 2 hours. Microspheres were then centrifuged, washed twice with water, and resuspended in 40 μl of phosphate-buffered saline (PBS), pH 7.4.

In other embodiments, nucleic acid analogs or target polynucleotides are attached to microspheres, as described by Running and Urdea, BIOTECHNIQUES 8:276 (1990).

Any target polynucleotide, or group of target polynucleotides, may be detected using the solid substrate-based system. In this case, a solid substrate contains multiple nucleic acid analogs or target polynucleotides immobilized on a solid substrate. A negative control nucleic acid analog or target polynucleotide that does not form nucleic acid analog/polynucleotide hybrid is preferably included on the solid substrate. A positive control nucleic acid analog that always forms a nucleic acid analog/polynucleotide hybrid is preferably included on the solid substrate.

An end of a monofilament can also serve as a solid substrate. In such an embodiment, a nucleic acid analog is preferably immobilized on the end of a monofilament, and a dye and target polynucleotide are preferably added to the tip. When light is applied to the end of the monofilament, the change in an optical property of the dye is monitored over time.

Fluorescent nanobeads such as quantum dots (Invitrogen Corporation, Carlsbad, Calif.) can serve as the solid substrate. Quantum dots cores are composed of semiconductor cadmium salts that fluoresce at different wavelengths based on salt and size. This core is enveloped in a shell that is composed of a non-emissive transparent, but structurally related, material that can be efficiently wed to the underlying core material. In this way, the core molecules are tricked into sensing what still appears to be a virtually infinite array of atoms in every direction; little or no reorganization is necessary and the fluorescent emission remains stable and bright over a long period of time.

In another preferred embodiment, nucleic acid analogs are preferably conjugated to quantum dots that emit at different wavelengths. A single sequence can be conjugated to dots of a single wavelength or one or more sequences can be conjugated to dots of a single wavelength. These quantum dots of either a single or multiple wavelengths are preferably mixed with a polynucleotide mixture allowing hybrids to form. The dots with hybrids attached may be washed to remove non-complementary polynucleotide. A mixture with dye and reaction buffer is added to the dots. The dots with hybrid are then dispensed in fraction wells of a multiwell plate as described at U.S. Pat. No. 6,838,243. In each well there are many fraction wells. The fraction wells are of the correct size that each can accommodate a single dot. The amount of liquid dispersed is such that the fraction wells are not quite full. The mixture is then exposed to an activating light. After a period of time (or at multiple times) the colors/fluorescence of the fraction wells are determined. The fluorescence of the quantum dot identifies the target sequence and the disappearance of an optical property in the reaction indicates the presence of the target sequence in the polynucleotide mixture.

The reactions can be quantitative based on the number of quantum dots bioconjugated with a given sequence indicating the presence of that sequence. Other types of beads may be used, including but not limited to Luminex® beads (MiraiBio, Alameda, Calif.). In some embodiments, beads may be magnetic to assist in the washing. Centrifugation may be involved in the washing.

In certain embodiments, a solid support with a specific background color provides significantly improved detection of the target polynucleotide. Specific embodiments of solid supports can have any color background known in the art. In one particular embodiment, the solid support has a white background. The solid support can be a microtiter plate having a specific color of well, such as a microtiter plate having a white background. Alternatively, the compositions can include a sample surface modified to have the same interior as any plate described herein.

C. Gel-Based Formats

The methods disclosed herein can be used to determine the presence or amount of a target polynucleotide in a gel based assay. A gel, such as an agarose or acrylamide gel, that contains the dye is prepared. A sample that may contain, does not contain, or is suspected of containing, or suspected of not containing, a target polynucleotide is added to the gel. The components of the sample can be separated from each other on the gel. A nucleic acid analog that is complementary to a target nucleic acid sequence of a target polynucleotide is added either before or after digestion of the sample polynucleotide but before an electric current is applied to the gel. Alternatively, the nucleic acid analog can be added after an electric field is applied to the gel. After the bands are allowed to migrate on the gel, a light stimulus is applied to the gel.

After the light stimulus is applied to the gel, the portion of the gel containing the NAA/TP hybrid has at least one different optical property relative to the rest of the gel. In one exemplary embodiment, when the gel is combined with 3,3′-diethylthiacarbocyanine iodide dye and exposed to a light stimulus, visually the resulting gel lacks any color (apart from the agarose-colored portions) where the NAA/TP hybrid is present. The remaining portion of the gel has a detectable color. In addition, by illumination with a 254 nm transilluminator, the gel will fluoresce except where the NAA/TP hybrid is absent. In the region of the NAA/TP hybrid, there is no fluorescence. The presence of a target nucleic acid sequence in a target polynucleotide is thereby identified on a gel.

The ability to determine the presence of a target polynucleotide sequence on a gel can be adapted to any number of conventional molecular biology techniques. For example, a conventional Southern blot can be adapted to the methods of determining the presence of a target polynucleotide on a gel. Target polynucleotides are digested by a conventional restriction digest and run on a conventional agarose gel that contains dye. A nucleic acid analog can be added to the target polynucleotides before, during, and/or after restriction digestion. In certain formats, the target nucleic acids can be denatured. Instead of transferring the polynucleotides to a membrane, the gel is exposed to light stimulus. The loss of fluorescence intensity of the dye corresponds to the location of a target polynucleotide containing the target nucleic acid sequence. In an alternative method, the nucleic acid analog is added to the gel. In various non-limiting embodiments, both the nucleic acid analog and the dye are added to the gel after the polynucleotides have been separated by the electric current. The electrically separated polynucleotides can be transferred to a membrane and the nucleic acid analog allowed to hybridize to the immobilized polynucleotides that have the complementary sequence. This membrane is then immersed in liquid containing dye and other reaction components, after some period of time the membrane is removed from the liquid and exposed to light stimulus. The presence of the complementary polynucleotide would be indicated by a clear spot on the membrane.

Unlike conventional Southern blot analysis, the presently described method does not require heat or salt-based denaturation of the nucleic acid, and does not require blotting to identify target polynucleotides having a specific nucleic acid sequence, and does not require hybridization of the probe to the transferred polynucleotides.

The methods disclosed herein can also be adapted to a northwestern blot. A “Northwestern” analysis is the generic term for studying protein-RNA interactions in either a solution-phase format, a gel (electrophoresis) format, or a substrate (such as a membrane) blot format. A protein may bind to RNA in a sequence-specific manner (e.g., HIV's tat protein binding to the TAR sequence) or in a sequence-independent manner (e.g., RNA-binding proteins). The methods can be used to identify specific sequences within RNA that are relevant to protein binding. For example, into a solution containing a RNA segment and a protein (thought to bind to a particular RNA sequence) is added a complementary nucleic analog and the dye. Upon exposure to light, the solution either changes color indicating that the nucleic acid analog hybridized to the RNA and the protein did not, or the solution does not change color indicating that the protein has bound to the RNA effectively blocking the nucleic acid analog from hybridizing.

The same methods can be performed in a gel electrophoresis format where the gel contains the dye. The gel is stained post electrophoresis. The sample containing the RNA, the protein, and the nucleic acid analog could be electrophoresed in a gel matrix followed by exposure of the gel to light. Identification of the band corresponding to nucleic acid analog hybridizing to the RNA is visualized by a loss of color (or “hole”) in the gel. Absence of the “hole” would be indicative of protein-RNA interaction in a sequence-specific manner, thereby inhibiting binding of the NAA.

Similarly, for substrate blot experiments, the protein (or RNA) could be immobilized to a substrate, followed by incubations with RNA sequences (or protein), followed by incubations with the nucleic acid analog and dye, followed by exposure to light. The loss of color would indicate hybridization of the NAA at a specific RNA sequence not bound to the protein. The presence of color would indicate a protein-RNA interaction at the specific sequence inhibiting binding of the NAA.

Modification of a nucleic acid analog with a positively-charged molecule can be used to increase specificity of hybridization. For example, a nucleic acid analog can be modified to contain a positive charge or linked to a molecule or molecules having a positive charge. The positive current in a gel directs the positively charged nucleic acid analog and the target polynucleotide in opposite directions. The greater the number of hydrogen bonds between the nucleic acid analog bases and the target polynucleotide bases, the greater the likelihood that the NAA/TP hybrid will remain annealed. Depending on the current and potential applied across the gel, NAA/TP hybrids containing mismatched sequences can be pulled apart, resulting in denaturation of the NAA/TP hybrid. The absence of a hybrid will result in a baseline change in an optical property within/of the gel.

The methods of determining the presence or quantity of a target polynucleotide on a gel can be adapted to any gel-based method. For example, determining the presence of a target polynucleotide on a gel can be adapted to conventional Northern blot analysis, in which the target polynucleotide is RNA, not DNA. The methods of determining the presence or quantity of a target polynucleotide on a gel also can be adapted to northwestern analysis. Conventional methods are further disclosed, for example, in Molecular Cloning: A Laboratory Manual, third edition (Sambrook et al., 2000) Cold Spring Harbor Press and Molecular Cloning: A Laboratory Manual, second edition (Sambrook et al., 1989) Cold Spring Harbor Press, both of which are incorporated herein by reference in their entirety.

D. Detection of Polynucleotides Involved in Genetic Manipulation.

The different rate of change of an optical property of a mixture (or gel) in the presence of an NAA/TP hybrid can be used to screen for transformation of bacterial colonies in a colony dot-blot. In conventional dot-blot assays, putative transformed cells are grown into colonies on medium. The colonies are transferred to a membrane, their location is fixed, the cells are lysed, and the polynucleotides are attached to the membrane.

The dot blot can be adapted using the methods disclosed herein to determine the presence of target polynucleotide in transformed phage, other viral particles, genetic material, or detection of transfection or infection of eukaryotic cells. The detection of the target polynucleotide attached to the membrane could occur via several methods. In one non-limiting example, a membrane is washed and a nucleic acid analog that is complementary to the sequence of interest or designed to show disruption of the sequence of interest. After a wash step (s) the membrane is placed on a gel-based film that contains the dye. This sandwich is exposed to a light stimulus. Areas in which color is reduced or disappears (or fluorescent emission is reduced or disappears) indicate the presence of the NAA/TP hybrid and thus the polynucleotide sequence of interest.

In another variation, the nucleic acid analog and the dye may be both in the stationary phase of the gel and simultaneously sandwiched to the membrane with the attached polynucleotides. In yet another variation, the dye (with or without the nucleic acid analog) can be in a liquid gel that is poured on the membrane and areas in which color or fluorescent emission is reduced or gone after exposure to light stimulus indicate the presence (and location) of colonies with the sequence of interest. In a further variation, the colonies may not be transferred to a membrane, but are preferably rather lysed on the plate and any of the above detection schemes applied. In a yet further variation, the polynucleotides may never be attached to the membrane and allowed to interact with reaction components held in a stationary phase (such as a gel).

Alternatively the colonies may be picked into a reaction vessel and the reactions with the change in an optical property of a dye occur in the reaction vessel.

### V. Quantifying the Amount of a Target Polynucleotide

The methods and compositions disclosed herein may be used to quantify the amount of target polynucleotide in a sample. In one embodiment, the amount of a target polynucleotide may be detected by establishing serial dilutions of the nucleic acid analog molecule, adding various amounts of the target polynucleotide samples, and comparing the samples to controls of known concentrations. In another embodiment, the amount of a target polynucleotide may be detected by establishing serial dilutions of the target polynucleotide, adding various amounts of the nucleic acid analogs or target polynucleotides, and comparing the samples to controls of known concentrations.

Alternatively, the amount of a target polynucleotide can be detected by measuring the kinetics of the assay based on time. Measurements of the dye in the combined mixture are taken at regular intervals after preparation of the mixture, or after application of light stimulus. The dye may be detected at distinct times after combination of the mixture, or after application of the light stimulus. The time may be any fixed time, for example the total time for the change in optical property, or the time required for the optical property to have changed by a certain percentage, such as, but not limited to, about 20%. The reactions can be frozen (further change stopped), for example with the addition of solvents such as 20% methanol, 15% isopropanol, 15% DMSO, or 10% butanol.

The quantity of polynucleotide in a sample may be determined after exposure to the light stimulus. The change in the optical property of the dye may be measured following pre-exposure to the light stimulus for the starting optical property. Measurements may be taken at distinct times (for example, but not limited to, taken at 30 second intervals, 1 second intervals, millisecond intervals, or microsecond intervals) after exposure to the light stimulus. The reactions can be frozen (further change stopped) as described above.

Changes in the sample due to exposure to the light stimulus can be observed in several ways. The change in the optical property may be observed as a change in color, absorbance, transmittance, fluorescence, reflectance, chemiluminescence, or a combination thereof. Alternatively, the change in optical property can be read using a reader. This change is measured using a spectrophotometer or a fluorometer, such as Tecan Genios or a Tecan Safire. Specific observation wavelengths may be selected, for example by a filter. A positive control expresses a change in absorbance faster than a negative test. It can be measured as a difference in the rate of change, or the difference in the change at a set time. If a light stimulus is used and fluorescent properties are observed, the light stimulus provided to the sample is at a higher energy (lower wavelength) than the observed emission. The excitation may be at, for example, 535 nm and the emission may be read at 590 nm. The fluorescence may be measured as a difference in the rate of change or the difference in the change at a set time or at a minimum time.

### VI. Target Binding Complexes

The methods and associated compositions disclosed herein can also be used as a reporter to facilitate identification of molecular interactions, such as protein-protein interactions or protein-glycoprotein interactions.

A target binding complex includes a target binding component and a reporter complex. Target binding components, discussed in more detail below, are any molecule that is capable of binding a target. A reporter complex includes a first reporter nucleotide sequence, a second reporter nucleotide sequence, and a dye. At least one component of the reporter complex is covalently bonded to a target binding component to form a modified target binding component. The modified target binding component is introduced to a sample suspected of containing a target. Preferably, washing steps are performed to remove target binding component that is not bound to the target. The remaining components of the reporter complex are added to form a target binding complex. The order of addition of the target binding complex or the remaining components of the reporter complex to the sample is not critical, and can be in reverse order. Optionally, a light stimulus is provided; preferably, the light stimulus is provided. The rate of change in an optical property of the sample (now including the dye) is determined, as described above for NAA/TP hybrids. The presence or amount of the target is thereby determined.

It will be understood that any other components described in the methods in the present application can be added to the target binding complex in any combination.

Further, it will be understood that methods of detecting targets can be performed using any format disclosed herein. For example, the target-binding complex can be adapted to a liquid-based format, solid-based format, or gel-based format. Non-limiting examples of formats include gel matrix platforms, electrophoretic platforms, membrane-bound platforms, chromatographic platforms, immobilized plates, and immobilized beads.

A. Reporter Complex

The reporter complex is similar to, and in some cases the same as, the combination of target polynucleotide, nucleic acid analog, and dye disclosed in the methods of detecting a target polynucleotide. As noted above, the reporter complex includes a first reporter nucleotide sequence, a second reporter nucleotide sequence, and a dye. The first reporter nucleotide sequence can be a DNA, RNA, or a nucleic acid analog. Likewise, the second reporter nucleotide sequence can be, independently of the first reporter nucleotide, a DNA, RNA, or a nucleic acid analog. The first and second reporter nucleotide sequences hybridize to form a double-stranded hybrid.

In certain embodiments, the first reporter nucleotide sequence is covalently bonded to the target-binding component. The second reporter nucleotide sequence and dye are subsequently added to form the mixture. The order in which the second reporter nucleotide sequence and dye are added is not critical.

Alternatively, the first reporter nucleotide sequence and second reporter nucleotide sequence can be covalently linked in a 5′-3′ arrangement to form a self-hybridizing hairpin, or can be crosslinked. The hairpin or crosslinking stabilizes hybrid formation, and minimizes loss of single-stranded polynucleotide. In this embodiment, the dye is then added to form the target binding complex. In another alternative, the dye can be covalently bonded to the first reporter nucleotide sequence or the second reporter nucleotide sequence.

In certain embodiments, the first reporter nucleotide sequence and/or the second reporter nucleotide sequence can have a non-complementary overhang. It will be understood that the first and second reporter nucleotide sequences are complementary while still maintaining one or more overhangs.

The components of the reporter complex can be covalently linked to the target binding component by any means known in the art. The functional groups of amino acids of ligands suitable for covalent binding under mild conditions include but are not limited to (i) the alpha amino groups of the chain and the epsilon amino groups of lysine and arginine, (ii) the alpha carboxyl group of the chain end and the beta and gamma carboxyl groups of aspartic and glutamic acids, (iii) the phenol ring of tyrosine, (iv) the thiol group of cysteine, (v) the hydroxyl groups of serine and threonine, (vi) the imidazile group of histidine, and (vii) the indole group of tryptophan. Similarly, the nucleic acid analog can be synthesized to contain any of these chemical moieties.

Those skilled in the art will recognize any number of established chemical conjugating techniques for covalently attaching nucleic acid analogs to ligands. Some non-limiting examples include □ yanogens bromide formation of reactive cyclic-imido carbamate for covalent coupling of amines, carbodiimide formation of O-acyl isourea for coupling of amines, malimidobenzoyl NHS ester formation for coupling of amines and sulfhydryls, and malimidocaproic acid hydrazide HCl for coupling of sulfhydryls and carbohydrates. Non-covalent attachments can be accomplished by creating oxidized disulfide bonds between cysteines from nucleic acid analogs and ligands. These can be easily reduced by dithiothreitol (DTT) or tris(2-carboxyethyl)phosphine (TCEP) thereby releasing the nucleic acid analog/polynucleotide hybrid from the ligand as needed.

The target binding component can be covalently attached to the at least one component of the reporter complex by a linking group. Linking groups are chemical moieties that link or connect reactive groups to [[Ps. The linking group can be any linking group can include one or more alkyl groups such as methyl, ethyl, propyl, butyl, etc. groups, alkoxy groups, alkenyl groups, alkynyl groups or amino group substituted by alkyl groups, cycloalkyl groups, polycyclic groups, aryl groups, polyaryl groups, substituted aryl groups, heteroaryl groups, and substituted heteroaryl groups. Linking groups may also comprise poly ethoxy aminoacids such as AEA ((2-amino)ethoxy acetic acid) or a preferred linking group AEEA ([2-(2-amino)ethoxy)]ethoxy acetic acid).

The sequence of the first and second reporter nucleotide sequences may be designed in a variety of ways. By way of example and not limitation, different reporter nucleotide sequence sequences may be optimized to produce an increased rate of change in optical property of a sample that includes the dye. Empirically determined “universal” first and second reporter nucleotide sequences will be used to maximally meet assay requirements.

A series of first and second reporter nucleotide sequences can be prepared by any method known in the art. For example, the reporter nucleotide sequences can be synthesized in vitro or in vivo (such as by recombinant methods). After the reporter nucleotide sequences are annealed, the rate of change of the sample that includes the dye can be tested under a number of different conditions. The greater the rate of change for first and second reporter nucleotide sequences have a specific sequence, the when the first and second reporter nucleotide sequences are covalently linked in a 5′ to 3′ arrangement.

The first and second reporter nucleotide sequence sequences can be of any length, provided that they hybridize together. The length of the first and second reporter nucleotide sequences also can be optimized, as discussed above.

The optimization methods can be adapted for any embodiment of first reporter nucleotide sequence, second reporter nucleotide sequence, and dye. For example, the first or second reporter nucleotide sequences can be covalently bonded to the target binding component, and the assay rate of change in the assay can be determined. The dye can be covalently bound to the target binding component.

It will be understood that screening methods can be optimized for the addition of any compound disclosed in the present application.

B. Target Binding Components

The target binding component may be any molecule that is capable of selectively interacting with a desired target. Exemplary targets include, but are not limited to, cells, microorganisms (such as bacteria, fungi, and viruses), polypeptides, nucleic acids (such as polynucleotides, cDNA molecules, or genomic DNA fragments), hormones, cytokines, drug molecules, carbohydrates, pesticides, dyes, amino acids, or small organic or inorganic molecules. Target binding components having limited cross-reactivity are generally preferred. Exemplary target binding components include, for example, antibodies, antibody fragments, non-antibody receptor molecules, template imprinted materials, lectins, enzymes, and organic or inorganic binding elements.

Some of the specific embodiments of target binding components are explained in more detail below. This disclosure does not limit the scope of the target binding components, as used herein.

### 1. Antibodies and Antibody Fragments

In certain embodiments, the target binding component may be an antibody or an antibody fragment. For example, target binding components may be monoclonal antibodies, or derivatives or analogs thereof, including without limitation: Fv fragments, single chain Fv (scFv) fragments, Fab′ fragments, F(ab′)₂ fragments, single domain antibodies, camelized antibodies and fragments thereof, humanized antibodies and fragments thereof, and multivalent versions of the foregoing. Multivalent target binding components include without limitation: monospecific or bispecific antibodies, such as disulfide stabilized Fv fragments, scFv tandems ((ScFV)₂ fragments), diabodies, tribodies or tetrabodies, which typically are covalently linked or otherwise stabilized (e.g., leucine zipper or helix stabilized) scFv fragments; receptor molecules that naturally interact with a desired target molecule.

In one embodiment, the target binding component is preferably an antibody. Preparation of antibodies may be accomplished by any number of well-known methods. For generating monoclonal antibodies, presuming that the antigen of interest is known and available, the first step is immunization of animals, typically mice, with a desired antigen (e.g., a desired target molecule or fragment thereof). Once the mice have been immunized, and preferably boosted one or more times with the desired antigen(s), monoclonal antibody-producing hybridomas are preferably prepared and screened according to well-known methods (see, e.g., Kuby, Janis, IMMUNOLOGY, Third Edition, pp. 131-139, W.H. Freeman & Co. (1997), for a general overview of monoclonal antibody production, that portion of which is incorporated herein by reference).

In vitro methods that combine antibody recognition and phage display techniques allow one to amplify and select antibodies with very specific binding capabilities. See, e.g., Holt et al., Current Opinion in Biotechnology 11:445 (2000). These methods typically are much less cumbersome than preparation of hybridomas by traditional monoclonal antibody preparation methods. Binding epitopes may range in size from small organic compounds such as bromo uridine and phosphotyrosine to oligopeptides on the order of 7-9 amino acids in length.

In another embodiment, the target binding component may be an antibody fragment. Preparation of antibody fragments may be accomplished by any number of well-known methods. In one embodiment, phage display technology may be used to generate antibody fragment target binding components that are specific for a desired target molecule, including, for example, Fab fragments, Fv's with an engineered intermolecular disulfide bond to stabilize the VH-VL pair, scFvs, or diabody fragments. As an example, production of scFv antibody fragments using phage display is described below.

For phage display, an immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired VH and VL chains, to yield cDNA. The desired VH and VL chains are amplified by polymerase chain reaction (PCR) typically using VH and VL specific primer sets, and are ligated together, separated by a linker. VH and VL specific primer sets are commercially available, for instance from Stratagene, Inc. of La Jolla, Calif.

Assembled VH-linker-VL product (encoding an scFv fragment) is selected for and amplified by PCR. Restriction sites are introduced into the ends of the VH-linker-VL product by PCR with primers including restriction sites and the scFv fragment is inserted into a suitable expression vector (typically a plasmid) for phage display. Other fragments, such as a Fab′ fragment, may be cloned into phage display vectors for surface expression on phage particles. The phage may be any phage, such as lambda, but typically is a filamentous phage, such as fd and Ml 3, typically M13.

In phage display vectors, the VH-linker-VL sequence is cloned into a phage surface protein (for M13, the surface proteins g3p (pIII) or g8p, most typically g3p). Phage display systems also include phagemid systems, which are based on a phagemid plasmid vector containing the phage surface protein genes (for example, g3p and g8p of Ml 3) and the phage origin of replication. To produce phage particles, cells containing the phagemid are rescued with helper phage providing the remaining proteins needed for the generation of phage. Only the phagemid vector is packaged in the resulting phage particles because replication of the phagemid is grossly favored over replication of the helper phage DNA. Phagemid packaging systems for production of antibodies are commercially available. One example of a commercially available phagemid packaging system that also permits production of soluble ScFv fragments in bacteria cells is the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Phage display systems, their construction and screening methods are described in detail in, among others, U.S. Pat. Nos. 5,702,892, 5,750,373, 5,821,047, and 6,127,132.

Typically, once phage are produced that display a desired antibody fragment, epitope specific phage are selected by their affinity for the desired immunogen and, optionally, their lack of affinity to compounds containing certain other structural features. A variety of methods may be used for physically separating immunogen-binding phage from non-binding phage. Typically the immunogen is fixed to a surface and the phage are contacted with the surface. Non-binding phage are washed away while binding phage remain bound. Bound phage are later eluted and are used to re-infect cells to amplify the selected species. A number of rounds of affinity selection typically are used, often increasingly higher stringency washes, to amplify immunogen binding phage of increasing affinity. Negative selection techniques also may be used to select for lack of binding to a desired target. In that case, un-bound (washed) phage are amplified.

Although it is preferred to use spleen cells and/or B-lymphocytes from animals pre-immunized with a desired immunogen as a source of cDNA from which the sequences of the VH and VL chains are amplified by RT-PCR, naive (un-immunized with the target immunogen) splenocytes and/or B-cells may be used as a source of cDNA to produce a polyclonal set of VH and VL chains that are selected in vitro by affinity, typically by the above-described phage display (phagemid) method. When naive B-cells are used, during affinity selection, the washing of the first selection step typically is of very low stringency so as to avoid loss of any single clone that may be present in very low copy number in the polyclonal phage library. By this naive method, B-cells may be obtained from any polyclonal source. B-cell or splenocyte cDNA libraries also are a source of cDNA from which the VH and VL chains may be amplified. For example, suitable murine and human B-cell, lymphocyte and splenocyte cDNA libraries are commercially available from Stratagene, Inc. and from Clontech Laboratories, Inc. of Palo Alto, Calif. Phagemid antibody libraries and related screening services are provided commercially by Cambridge Antibody Technology of the U.K. or MorphoSys USA, Inc.,) of Charlotte, N.C.

The target binding components do not have to originate from biological sources, such as from naive or immunized immune cells of animals or humans. The target binding components may be screened from a combinatorial library of synthetic peptides. One such method is described in U.S. Pat. No. 5,948,635, which described the production of phagemid libraries having random amino acid insertions in the pIII gene of Ml 3. These phage may be clonally amplified by affinity selection as described above.

Panning in a culture dish or flask is one way to physically separate binding phage from non-binding phage. Panning may be carried out in 96 well plates in which desired immunogen structures have been immobilized. Functionalized 96 well plates, typically used as ELISA plates, may be purchased from Pierce Biotechnology, Inc. of Rockford, Ill. Polypeptide immunogens may be synthesized directly on NH₂ or COOH functionalized plates in an N-terminal to C-terminal direction. Other affinity methods for isolating phage having a desired specificity include affixing the immunogen to beads. The beads may be placed in a column and phage may be bound to the column, washed and eluted according to standard procedures. Alternatively, the beads may be magnetic so as to permit magnetic separation of the binding particles from the non-binding particles. The immunogen also may be affixed to a porous membrane or matrix, permitting easy washing and elution of the binding phage.

In certain embodiments, it may be desirable to increase the specificity of the target binding component for a given target molecule using a negative selection step in the affinity selection process. For example, target binding component displaying phage may be contacted with a surface functionalized with immunogens distinct from the target molecule. Phage are washed from the surface and non-binding phage are grown to clonally expand the population of non-binding phage thereby de-selecting phage that are not specific for the desired target molecule. In certain—embodiments, random synthetic peptides may be used in the negative selection step. In other embodiments, one or more immunogens having structural similarity to the target molecule may be used in the negative selection step. For example, for a target molecule comprising a polypeptide, structurally similar immunogens may be polypeptides having conservative amino acid substitutions, including but not limited to the conservative substitution groups such as: (i) a charged group, consisting of Glu, Asp, Lys, Arg, and His, (ii) a positively-charged group, consisting of Lys, Arg, and His, (iii) a negatively-charged group, consisting of Glu and Asp, (iv) an aromatic group, consisting of Phe, Tyr, and Trp, (v) a nitrogen ring group, consisting of His and Trp, (vi) a large aliphatic nonpolar group, consisting of Val, Leu, and Ile, (vii) a slightly polar group, consisting of Met and Cys, (viii) a small-residue group, consisting of Ser, Thr, Asp, Asn, Gly, Ala, Glu, Gln, and Pro, (ix) an aliphatic group consisting of Val, Leu, Ile, Met, and Cys, and (x) a small hydroxyl group consisting of Ser and Thr. Conservative substitutions also may be determined by one or more methods, such as those used by the BLAST (Basic Local Alignment Search Tool) algorithm, such as a BLOSUM Substitution Scoring Matrix, such as the BLOSUM 62 matrix, and the like. A functional way to define common properties between individual amino acids is to analyze the normalized frequencies of amino acid changes between corresponding proteins of homologous organisms (Schulz and Schirmer, PRINCIPLES OF PROTEIN STRUCTURE: Springer Advanced Texts in Chemistry, Springer-Verlag, N.Y., 1990).

Screening of target binding components will best be accomplished by high throughput parallel selection, as described in Holt et al. Alternatively, high throughput parallel selection may be conducted by commercial entities, such as by Cambridge Antibody Technologies or MorphoSys USA, Inc.

Alternatively, selection of a desired target binding component-displaying phage may be carried out using the following method.

Step 1: Affinity purify phage under low stringency conditions for their ability to bind to an immunogen fixed to a solid support (for instance, beads in a column).

Step 2: Elute the bound phage and grow the eluted phage. Steps 1 and 2 may be repeated with more stringent washes in Step 1.

Step 3: Absorb the phage under moderate stringency with a given protein mixture digested with a proteolytic agent of interest. Wash away the unbound phage with a moderately stringent wash and grow the washed phage. Step 3 may be repeated with less stringent washes.

Step 4: Affinity purify phage under high stringency for their ability to bind to the immunogen fixed to a solid support. Elute the bound phage and grow the eluted phage.

Step 5: Plate the phage to select single plaques. Independently grow phage selected from each plaque and confirm the specificity to the desired immunogen.

This is a general guideline for the clonal expansion of immunogen-specific target binding components. Additional steps of varying stringency may be added at any stage to optimize the selection process, or steps may be omitted or re-ordered. One or more steps may be added where the phage population is selected for its inability to bind to other immunogens by absorption of the phage population with those other immunogens and amplification of the unbound phage population. That step may be performed at any stage, but typically would be performed after step 4.

In certain embodiments, it may be desirable to mutate the binding region of the target binding component and select for target binding components with superior binding characteristics as compared to the un-mutated target binding component. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity, as described above.

In other embodiments, the target binding components may be modified to make them more resistant to cleavage by proteases. For example, the stability of the target binding components of the present invention that comprise polypeptides may be increased by substituting one or more of the naturally occurring amino acids in the (L) configuration with D-amino acids. In various embodiments, at least 1%, 5%, 10%, 20%, 50%, 80%, 90% or 100% of the amino acid residues of the target binding components may be of the D configuration. The switch from L to D amino acids neutralizes the digestion capabilities of many of the ubiquitous peptidases found in the digestive tract. Alternatively, enhanced stability of the target binding components of the invention may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of the target binding components may be achieved by intercalating one or more dextrorotatory amino acids (such as, dextrorotatory phenylalanine or dextrorotatory tryptophan) between the amino acids of the target binding component. In exemplary embodiments, such modifications increase the protease resistance of the target binding components without affecting their activity or specificity of interaction with a desired target molecule.

In certain embodiments, the antibodies or variants thereof, may be modified to make them less immunogenic when administered to a subject. For example, if the subject is human, the antibody may be “humanized”; where the complementarity determining region(s) of the hybridoma-derived antibody has been transplanted into a human monoclonal antibody, for example as described in Jones et al., NATURE 321:522 (1986), Tempest et al. BIOTECHNOLOGY 9:266 (1991), and U.S. Pat. No. 6,407,213. Also, transgenic mice, or other mammals, may be used to express humanized antibodies. Such humanization may be partial or complete.

In another embodiment, the target binding component is a Fab fragment. Fab antibody fragments may be obtained by proteolysis of an immunoglobulin molecule using the protease papain. Papain digestion yields two identical antigen-binding fragments, termed “Fab fragments”, each with a single antigen-binding site, and a residual “Fc fragment”. In an exemplary embodiment, papain is first activated by reducing the sulfhydryl group in the active site with cysteine, mercaptoethanol or dithiothreitol. Heavy metals in the stock enzyme may be removed by chelation with EDTA (2 mM) to ensure maximum enzyme activity. Enzyme and substrate are normally mixed together in the ratio of 1:100 by weight. After incubation, the reaction can be stopped by irreversible alkylation of the thiol group with iodoacetamide or simply by dialysis. The completeness of the digestion should be monitored by SDS-PAGE and the various fractions separated by protein A-Sepharose or ion exchange chromatography.

In still another embodiment, the target binding component is an F(ab′)₂ fragment. F(ab′)₂ antibody fragments may be prepared from IgG molecules using limited proteolysis with the enzyme pepsin. Exemplary conditions for pepsin proteolysis are I00 times antibody excess w/w in acetate buffer at pH 4.5 and 37° C. Pepsin treatment of intact immunoglobulin molecules yields a F(ab′)₂ fragment that has two antigen-combining sites and is still capable of crosslinking antigen. Fab′ antibody fragments may be obtain'ed by reducing F(ab′)₂ fragments using mercaptoethylamine. The Fab′ fragments may be separated from unsplit F(ab′)₂ fragments and concentrated by application to a Sephadex G-25 column (M=46,000-58,000).

### 2. Non-Antibody Embodiments

In other embodiments, the target binding component may be a non-antibody receptor molecule, including, for example, receptors that naturally recognize a desired target molecule, receptors that have been modified to increase their specificity of interaction with a target molecule, receptor molecules that have been modified to interact with a desired target molecule not naturally recognized by the receptor, and fragments of such receptor molecules (see, e.g., Skerra, MOLECULAR RECOGNITION 13:167 (2000)).

### 3. Template Imprinting Materials

In other embodiments, the target binding components may be a template imprinted material. Template imprinted materials are structures that have an outer sugar layer and an underlying plasma-deposited layer. The outer sugar layer contains indentations or imprints that are complementary in shape to a desired target molecule or template so as to allow specific interaction between the template imprinted structure and the target molecule to which it is complementary. Template imprinting can be utilized on the surface of a variety of structures, including, for example, medical prostheses (such as artificial heart valves, artificial limb joints, contact lenses and stents), microchips (preferably silicon-based microchips), and components of diagnostic equipment designed to detect specific microorganisms, such as viruses or bacteria.

Template-imprinted materials are discussed in U.S. Pat. No. 6,131,580.

In another embodiment, a target binding component of the invention may be modified so that its rate of traversing the cellular membrane is increased. For example, the target binding component may be attached to a peptide that promotes “transcytosis,” e.g., uptake of a polypeptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, CELL 55:1179 (1989)).

Alternatively, the internalizing peptide may be derived from the Drosophila antennapedia protein, or homologs thereof. The 60 amino acid long homoeodomain of the homeo-protein antennapedia has been demonstrated to translocate through biological membranes and can facilitate the translocation of heterologous polypeptides to which it is coupled. Thus, target binding components may be fused to a peptide consisting of about amino acids 42-58 of Drosophila antennapedia or shorter fragments for transcytosis (Derossi et al., J. BIOL. CHEM. 271:18188 (1996); Derossi et al., J. BIOL. CHEM. 269:10444 (1994); and Perez et al., J. CELL Sci. 102:717 (1992). The transcytosis polypeptide may also be a non-naturally-occurring membrane-translocating sequence (MTS), such as the peptide sequences disclosed in U.S. Pat. No. 6,248,558.

In exemplary embodiments, the dissociation constant of the target binding component for a target molecule is optimized to allow real time monitoring of the presence and/or concentration of the analyte in a given patient, sample, or environment.

### 4. Lectins

The target binding component can be a lectin. Lectins are a class of carbohydrate-binding proteins found in plants, viruses, microorganisms and animals. Frequently, lectins are multimeric having two or more of non-covalently associated subunits. A lectin may contain two or more of the same subunit, such as Con A, or different subunits, such as Phaseolus vulgaris agglutinin. At least one component of the reporter complex can be covalently bonded to the lectin.

Because of the specificity that each lectin has toward a particular carbohydrate structure, oligosaccharides with identical sugar compositions can be distinguished or separated. Certain lectins will bind only to structures with mannose or glucose residues, while others may recognize only galactose residues. Certain other lectins require that the particular sugar be in a terminal non-reducing position in the oligosaccharide, while others can bind to sugars within the oligosaccharide chain. Some lectins do not discriminate between a and b anomers, while others require not only the correct anomeric structure, but a specific sequence of sugars for binding. The affinity between a lectin and its receptor may vary a great deal due to small changes in the carbohydrate structure of the receptor. All of these properties that are peculiar to lectins enable one to discriminate between structures, to isolate one glycoconjugate, cell, or virus from a mixture, or to study one process among several. Because virtually all biological membranes and cell walls contain glycoconjugates, all living organisms can be studied with lectins.

In certain embodiments, a target binding component can include a chemical handle that facilitates its isolation, immobilization, identification, or detection, additionally, or in the alternative, the chemical handle can serve to increase the solubility of the target binding component. In various embodiments, chemical handles may be a polypeptide, a polynucleotide, a carbohydrate, a polymer, or a chemical moiety, or combinations or variants thereof. In certain embodiments, exemplary chemical handles include glutathione S-transferase (GST), protein A, protein G, calmodulin-binding peptide, thioredoxin, maltose binding protein, HA, myc, poly arginine, poly His, poly His-Asp or FLAG tags. Additional exemplary chemical handles include polypeptides that alter protein localization in vivo, such as signal peptides, type III secretion system-targeting peptides, transcytosis domains, nuclear localization signals, and the like. In various embodiments, a target binding component of the invention may include one or more chemical handles, including multiple copies of the same chemical handle or two or more different chemical handles. It is also within the scope of the invention to include a linker (such as a polypeptide sequence or a chemical moiety) between a target binding component of the invention and the chemical handle in order to facilitate construction of the molecule or to optimize its structural constraints. In another embodiment, the target binding complex including a chemical handle may be constructed so as to contain protease cleavage sites between the chemical handle and the target binding component of the invention in order to remove the chemical handle. Examples of suitable endoproteases for removal of a chemical handle include Factor Xa and TEV proteases.

In other embodiments, the target binding component can be a drug, a putative drug, a drug target, or a putative drug target. Drugs are disclosed, for example, in the MERCK INDEX 13^{th} Ed. (2001).

It will be understood that the target binding components discussed above are only exemplary. Any other target binding component that binds a target can be used.

### VII. Compositions

The present application also contemplates compositions including components used in the methods disclosed herein.

In certain embodiments, the compositions include at least two of the components that can be used in the methods disclosed herein. By way of example and not limitation, the composition can include a dye and a specific nucleic acid analog. More specially, the composition can include a dye, and at least one of a chiral PNA, an LNA, a morpholino nucleic acid, a TNA, or a metal-linked nucleic acid. Such compositions can further include a surfactant and/or a target. In certain circumstances, alcohol is added in combination with a surfactant.

In other embodiments, the composition can include a detergent and at least one of the other components used in the method. For example, a composition can include a detergent and a nucleic acid analog, a dye, and/or a target polynucleotide. The detergent can be any detergent known in the art. In certain formulations, the detergent can be a cationic, anionic, non-ionic, or zwitterionic detergent. In certain formulations, the detergent can be at least one of TMAC, LSS, SDS, Tween® 20, Tween® 40, Tween® 80, NP40 Tergitol®, Span® 20, Span® 80, and CHAPS. In certain other formulations, the detergent can be at least one of Tween® 20, Tween® 40, Tween® 80, Tergitol® NP40, LSS, TMAC, and CHAPS. The composition can also include a PNA, an LNA, a morpholino nucleic acid, a TNA, or a metal-linked nucleic acid. The composition can include methanol, ethanol, isopropanol, butanol or other organic solvents known in the art.

The composition can also include a dye combined with a target polynucleotide and nucleic acid analog. For example, the composition can include a target polynucleotide/nucleic acid analog hybrid, combined with a dye. The composition can further include one or more of any other component disclosed in the methods herein.

The composition can also include a reagent that stops further change in the optical property of the dye. By way of example and not limitation, the stopping reagent can be a solvent such as 20% methanol, 15% isopropanol, 15% DMSO, or 10% butanol. If a surfactant is present for example, Tween® 80 at a concentration of about 0.05%, then a higher concentration of solvents is needed, such as in the range of about 40-50% methanol.

The composition can also include buffers, such as 5 mM phosphate buffer (pH 5.5).

In certain embodiments, a solid support with a specific background color provides significantly improved detection of the target polynucleotide. Compositions can include a solid surface with any color background, including a white background. The solid support, for example, can be a microtiter plate having a white background. Alternatively, the compositions can include a sample surface modified to have the same interior as any plate described herein.

The compositions can include any other compound, compounds, or device used in the methods disclosed herein, in any combination.

### VIII. Kits

In one aspect, the present application provides a kit for detecting target polynucleotides. A kit may include one or more reagents useful in the methods or compositions disclosed herein. For example, kits can include dyes, nucleic acid analogs (immobilized or not), surfactants, sources of light stimulus, buffers, alcohols, standards used for controls, keys illustrating positives and negatives of control samples for interpreting reaction results, and instructions. The kits may further include suitable packaging of the respective compositions and/or other optional components as disclosed below.

A. Dyes

The kits provided herein include one or more dyes. The dyes can include any dye disclosed herein. The dyes can be provided in pre-packages amounts, or can be provided in a single tube from which aliquots can be apportioned or diluted and then apportioned. The dyes may be further packaged in any suitable packaging for segregation from other components of the kit and to facilitate dispensing of the composition.

B. Nucleic Acid Analogs

The kits may also include one or more nucleic acid analogs. The nucleic acid analog may be any nucleic acid analog, as described herein. The nucleic acid analog may have any sequence that is complementary or fully complementary to a target nucleic acid sequence. The sequence may be any sequence known in the art. In one embodiment, the nucleic acid analog has a sequence disclosed herein.

The preferred kit contains one or more nucleic acid analog provided in any suitable container or containers (if the multiple nucleic acid analogs are packaged separately). The nucleic acid analog(s) may be pre-aliquoted into usable amounts, or provided in a single tube to be apportioned (with or without), or may be already immobilized on a solid surface. The container may be further packaged in any suitable packaging for segregation from other components of the kit and to facilitate dispensing aliquots. In another embodiment, two or more the nucleic acid analog sequences may be contained in the same package. Nucleic acid analogs having differing sequences can be a mixture in each tube, or they can be separately packaged two or more tubes, each with a single-sequence analog.

The kits may also include a vehicle to facilitate effective hybridization of the nucleic acid analog to the target polynucleotide, such as a non-specific carrier polynucleotide, or other compound, such as glycerol, or a vehicle that disrupts effective hybridization (possibly in the absence of surfactants), such as methanol, ethanol, butanol, DMSO, sodium hydroxide, and formamide.

C. Detergents

The kits preferably also include one or more detergents used in the methods and compositions disclosed herein. The detergent can be any detergent known in the art. In certain other formulations, the detergent can be a cationic, anionic, non-ionic, or zwitterionic surfactant. In certain formulations, the surfactant can be at least one of TMAC, LSS, SDS, Tween® 20, Tween® 40, Tween® 80, NP40 Tergitol®, Span® 20, Span® 80, and CHAPS. In still other formulations, the surfactant can be at least one of Tween® 20, Tween® 40, Tween® 80, Tergitol® NP40, LSS, TMAC, and CHAPS. The concentration of surfactant can be concentrated such that mixtures that include the surfactant have a specific concentration when diluted with other components.

The addition of alcohol to a mixture containing the detergent further reduces the photobleaching of the dye in the absence of an NAA/TP hybrid, but does not proportionally reduce the photobleaching of the dye in the presence of an NAA/TP hybrid. The aspect of the present invention is surprising because, in the absence of detergent, the addition of alcohol causes a greater photobleaching reduction in reactions with NAA/TP hybrid than in reactions without NAA/TP hybrid.

Any alcohol is preferably added so long as the added alcohol does not preclude the hybridization of the NAA to its complement, or otherwise preclude the catalytic activity of the NAA/NA hybrid. In certain embodiments, from about 8-12% ethanol or about 12-14% methanol is preferably added in tandem with a detergent at about 0.05%-0.5%.

D. Source of Light Stimulus

The kits are preferably outfilled with a source of light stimulus. The light source is preferably any light source known in the art. The light source can be capable of adjusting intensity and/or wavelength. Non-limiting examples of light sources include the Sylvania Cool White T8-CW, General Electric T8-C50, and Fritz Aurora 50/50, a Sylvania dulux S9W CF9DS/blue, Osram F9TT/50K, halogen autolamp, and SiC, InGaN, GaP, GaAsP, GaN+SiC, GaN-based Light Emitting Diodes, or solid-state lasers.

E. Polynucleotide Manipulating Components

The kits may also include components used to manipulate or preserve polynucleotides, such as buffers, enzymes, columns, and other materials.

The buffers, enzymes, columns, and other materials can include those that are used to lyse cells or extract DNA or RNA from a cell. The buffers, enzymes, columns, and other materials can also include components used to manipulate polynucleotides, including DNA and RNA. Such components include, for example, those disclosed in MOLECULAR CLONING: A LABORATORY MANUAL, third edition (Sambrook et al., 2000) Cold Spring Harbor Press, or any other reference disclosed herein.

F. Instructions

Kits preferably include instructions for performing the methods described herein. Instructions may be included as a separate insert and/or as part of the packaging or container, e.g., as a label affixed to a container or as writing or other communication integrated as part of a container. The instructions may inform the user of methods for application and/or removal of the contents of the kit, precautions and methods concerning handling of materials, expected results, warnings concerning improper use, and the like.

G. Additional Optional Components of the Kits

Kits may further contain components useful in practicing the methods disclosed herein. Exemplary additional components include chemical-resistant disposal bags, tubes, diluent, gloves, scissors, marking pens and eye protection.

The compositions can also include any type of solid surface described herein. The solid surface can be a specific color. In certain circumstances, the solid surface is white. The solid surface may, for example, be a microtiter plate having a number of white wells.

H. Computer Hardware and Software

The kits can also include computer hardware and/or computer software that can be used to measure the optical property of the mixture or gel or surface where the dye is included or placed. The hardware can include any detector used to measure the optical property. The software can include any algorithm used to note when a change in the optical property occurs, or determine a rate of change in the optical property. The kits can also include automated devices, such as those disclosed herein.

### IX. Sources of Target Polynucleotides

The methods, compositions, and kits described herein have a variety of uses. Non-limiting examples of these uses include detecting and quantifying organisms, including the subset thereof referred to as pathogens, toxins, and the like. Pathogens of interest that may be detected using the present invention include foodborne pathogens, environmental pathogens, waterborne pathogens, or pathogens implicated in bio- or agroterrorism. Other non-limiting uses include disease diagnosis, such as sexually transmitted disease diagnosis, detection of genes conferring antibiotic resistance, detection of genes conferring a predisposition for drug responses, detection of genes implicated in an effective drug response, detection of genetically-modified organisms, detection of non-indigenous fluora or fauna, detection of specific cancer-related genes, and mRNA levels. Additional non-limiting applications, relating, for example, to plant strain and/or grain quality, include agricultural applications and veterinary applications, many of which are the same or similar as the test developed for humans.

Examples, for illustration and not for limitation, are listed and described in the parent provisional application U.S. Ser. No. 60/655,929 (“the '929 application”), which is incorporated herein in its entirety. In particular, the '929 application sets forth detailed information regarding useful target polynucleotides, or descriptions of such polynucleotides, relating to pathogens (pp. 50-57), host response polynucleotides (pp. 57-58), foodborne and environmental pathogens (pp. 58-61), waterborne pathogens (pp. 61-62), bio- and agroterrorism (pp. 62-63), disease diagnostics such as genetic diseases and cancers (pp. 64-69), sexually-transmitted diseases (pp. 69-71), antibiotic resistance (pp. 71-72), genetic screening for a predisposition for drug responses (pp. 72-73), genes implicated in effective drug response (pp. 73-80), genetically-modified organisms (p. 80), nonidigenous flora and fauna (pp. 80-81), and agricultural and/or veterinary applications (pp. 81-83).

One particularly preferred embodiment of the present invention relates to methods, materials, and kits directed at the detection the tuberculosis pathogens, including Mycobacterium tuberculosis. Nucleic acid analogs may be designed to have sequences or fragments of sequences similar or identical to PCR primers used to identify tuberculosis. Examples of these PCR primers are disclosed in the art (see, e.g., M. J. Torres et al., DIAGN. MICROBIOL. INFECT. Dis. 45:207-12 (2003); B. Bhattacharya et al., TROP. MED. INT. HEALTH 8:150-7 (2003); M. Kafwabulula et al., INT. J. TUBERC. LUNG DIS. 6:732-7 (2002)).

In another particularly preferred embodiment, nucleic acid analogs are designed that bind to target polynucleotides common to an entire group of pathogens. For example, nucleic acid analogs may be designed to detect all bacteria (BP6) universal probe set, gram positive bacteria probe set (BP19), gram negative bacteria probe set (BP3), and Fungi probe set (FP8). Any sequence in a set may be used. Examples of the sequences of the nucleic acid analogs for BP6, BP19, BP3, and FP8 are shown below. S=G and C mixture, M=A and C mixture, Y=C and T mixture, and W=A and T mixture according to IUB codes for mixed base sites.

TABLE 2

| Pathogen | | | SEQ ID |
|---|---|---|---|
| Group | | Sequence | NO: |
| BP6 | oI2018 | 5′ gaaSSMYcYaacacYtagcact | 12 |
| | oI2019 | 5′ tacaaMgagYYgcWagacSgYgaS | 13 |
| BP19 | oI2021 | 5′ gcagYWaacgcattaagcact | 14 |
| | oI2022 | 5′ acgacacgagctgacgacaa | 15 |
| BP3 | oI2003 | 5′ tctagctggtctgagaggatgac | 16 |
| | oI2004 | 5′ gagttagccggtgcttcttct | 17 |
| FP8 | oI2055 | 5′ cctgcggcttaatttgactca | 18 |
| | oI2057 | 5′ tagcgacgggcggtgtgta | 19 |

The nucleic acid analogs can be used in clinical applications for the diagnosis of the microbial cause of sepsis or in other applications where the microbial content of products in important in evaluating their shelf-life and stability or other products where the sterility is being assessed.

Target polynucleotides may be specific to ribosomal RNA sequences, such as 16S RNA in E. coli. Ribosomal RNA contains specific sequences that are characteristic to their organism. By using nucleic acid analog sequences that are complementary or exactly complementary to a target polynucleotide characteristic of the ribosomal RNA sequence, pathogens may be identified based on their ribosomal RNA sequences. Ribosomal RNA sequences characteristic of different pathogens or strains of pathogens, may be found, for example, at D. J. Patel et al., J. MOL. BIOL. 272:645-664 (1997).

The foregoing examples, and those incorporated by reference from the '181 application, are presented here to illustrate the breadth of application for the present invention. Other examples of target polynucleotides usefully employed in the context of the present invention certainly exist, and more are identified daily as the natural result of scientific investigators attempting to understand the basis and develop cures for the many and various pathogens and diseases of humankind, plants, and animals. Additionally, target polynucleotides that measure the state of a locality's environment and o-her non-medica- or -veterinary or -agricultural applications are also contemplated as usefully employed with the present invention.

### EXAMPLES

The following non-limiting examples serve to more fully describe the manner of using the above-described methods and compositions. It is understood that these examples in no way serve to limit the scope of the subject matter described herein, but rather are presented for illustrative purposes.

All nucleic acid analog and DNA stock solutions in the following examples were made in 5 mM phosphate buffer, pH 5.5 unless otherwise noted. The 5 mM phosphate buffer, pH 5.5, was used as the reaction buffer in all examples, unless otherwise noted. The stock dye was made in methanol or DMSO.

### Example 1

This example illustrates uses and effects of different detergents on the diagnostic method of the present invention.

The addition of different classes of detergents at different concentrations was shown to have different effects on the signal to noise ratio. Some detergents increased the ratio while others had less dramatic effects. In one embodiment, detergent added to the reaction buffer increased the rate of change of the optical property in the test. In addition, each detergent reduced photobleaching of the dye in negative controls that lacked target polynucleotide.

Different detergents were added to the nucleic acid analog/target polynucleotide (NAA/TP) mixture, which included one of two target polynucleotides, one of two nucleic acid analogs, and a dye in a 5 mM phosphate buffer pH 5.5, at room temperature. Several different types of detergents were used, including cationic detergents, (specifically, tetramethyl ammonium chloride (“TMAC”)), anionic surfactants (specifically, N-lauroyl sarcosine sodium salt (“LSS”) and sodium dodecyl sulfate (“SDS”)), nonionic detergents (specifically, various polyethylene glycol sorbitan monooleate solutions, sold under the trade names Tween® 20, Tween® 40, and Tween® 80, a polyglycol ether detergent sold under the trade name Tergitol® NP-40; sorbitan monolaurate, sold under the trade name Span® 20; and sorbitane monooleate, sold under the trade name Span® 80), and zwitterionic detergents (specifically, 3-[(3-cholamidopropyl)dimethylammonio]-1-propanesulfonate, commonly known by its acronym “CHAPS”). The various detergents were purchased from Sigma-Aldrich, St. Louis, Mo. The term “detergent” is used herein synonymously with the term “surfactant”.

Aliquots of buffer reaction (each 5 mM phosphate, pH 5.5) containing different surfactants were prepared, and NAA/TP mixtures with the surfactants individually added were tested for the ability to (i) increase the sensitivity of the reaction and, separately, (ii) reduce photobleaching activity of the dye in a negative control. Each experiment contained four wells: (1) test well (non-chiral PNA, target polynucleotide, 3,3′-diethylthiacarbocyanine iodide dye and reaction buffer, with surfactant); (2) negative control well (non-chiral PNA, non-targeted polynucleotide, 3,3′-diethylthiacarbocyanine iodide dye, and reaction buffer, with and without surfactant); (3) dye control well (3,3′-diethylthiacarbocyanine iodide dye and reaction buffer, with and without surfactant); and (4) buffer control well (50 μl of reaction buffer, with and without surfactant). 10 pmoles 16S ncPNA (5′ ACTGCTGCCTCCCGTAG 3′ [SEQ ID NO:8] or 5′ TGCCTCCCGTAG 3′ [SEQ ID NO:9]), 10 pmoles of complementary oligo (5′ CTACGGGAGGCAGCAGT 3′ [SEQ ID NO: 10] or 5′ CTACGGGAGGC 3′ [SEQ ID NO: 11]), and 4 nmoles of 3,3′-diethylthiacarbocyanine iodide (“DTCC”; Sigma-Aldrich, St. Louis, Mo.) were placed in a 50 μl total reaction volume. ncPNAs (Applied Biosystems, Foster City, Calif.) and oligos (Sigma Genosys, St. Louis, Mo.) were reconstituted in water that was both DNase- and RNase-free as 100 μM stocks and further diluted to 2 μM working stocks. The DTCC dye was dissolved in dimethyl sulfoxide (“DMSO”; Sigma-Aldrich, St. Louis, Mo.) as an 8 mM stock. This stock was further diluted to a 2 mM working stock in 5 mM phosphate buffer (pH 5.5).

An initial fluorescence measurement was taken at time zero using a Tecan Genios microplate reader with the wavelengths set at 535 nm (for excitation) and 590 nm (for emission). The mixtures were then exposed to a light stimulus using the Aurora 50/50 (Fritz Industries, Inc., Mesquite, Tex.) for 1 minute intervals with fluorescence readings taken after each exposure for 5 minutes. For each reaction, the fluorescent emission values from the test well were compared to the fluorescent emission value from the negative control well (containing only ncPNA, dye, and reaction buffer) and converted to the percent changed. The percent change is calculated by the equation

100−[(RFU_{TW})÷(RFU_{NC})]×100,

where RFU_{TW} represents the fluorescent emission in relative fluorescent units of a reaction mixture in a test well that includes all required components of the test and RFU_{NC} represents the fluorescent emission in relative fluorescent units of a reaction mixture in a negative control well that does not include a target polynucleotide. The percent change is calculated at each time point. The percent difference between the measured rate in samples containing the target polynucleotide and the measured rate in samples not containing target polynucleotide was used to indicated the relative ability to detect presence of the target polynucleotide sequence.

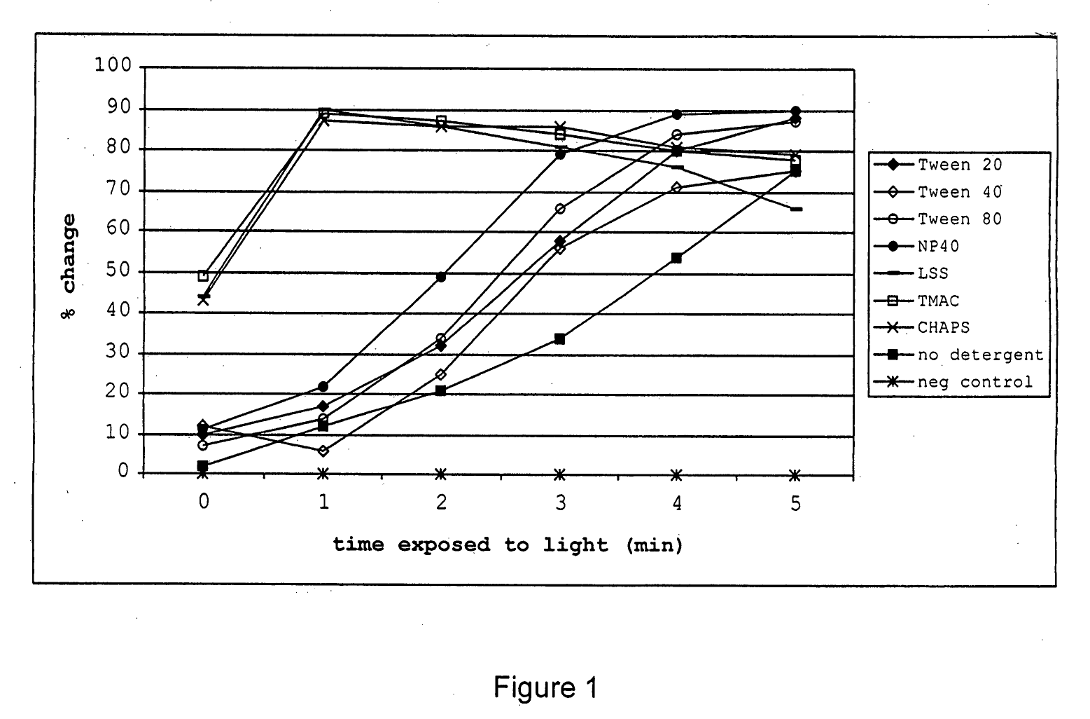

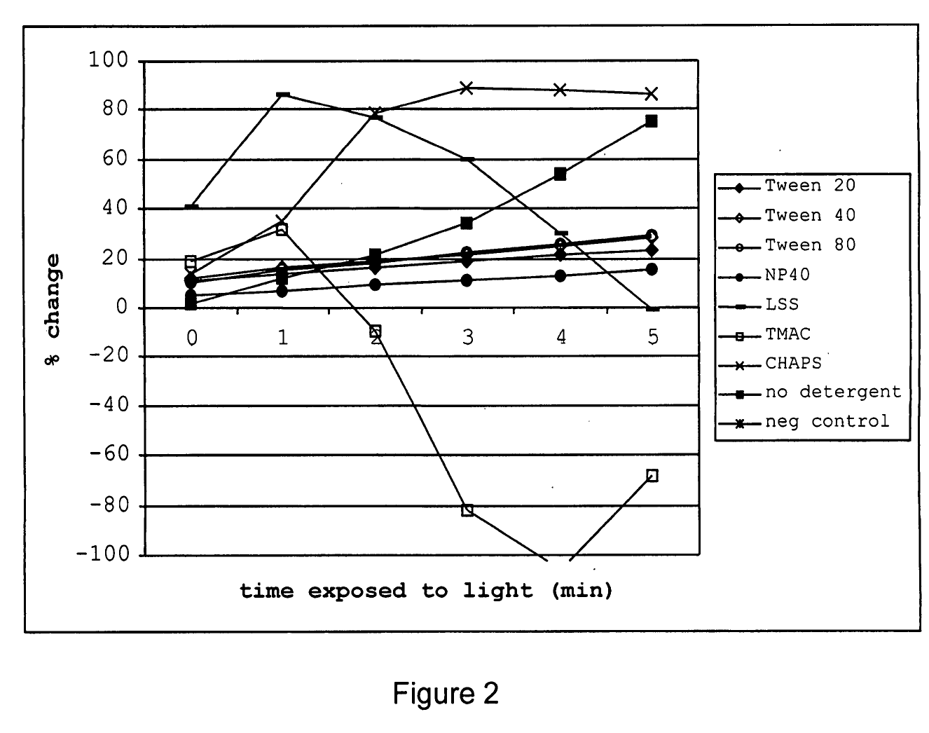

FIG. 1 graphically presents the data collected in the studies detailed here. The reactions were each conducted in the presence of a detergent having a concentration of 0.05% v/v. Detergents added to the reaction buffer included the nonionic detergents Tween®20 (♦), Tween® 40 (⋄), Tween® 80 (∘), and Tergitol® NP-40 (); the anionic detergent LSS (); the cationic detergent, TMAC (□); and the zwitterionic detergent CHAPS (×); where the symbols identified parenthetically after each of the named detergents are those used in the graphs of FIGS. 1 and 2. Improved sensitivity resulted upon the addition of 0.05% of any of the detergents when compared to the absence of detergent added. Decreased change in fluorescent emission of the dye in the absence of a nucleic acid analog/target polynucleotide hybrid compared to the dye alone was observed with the addition of Tween® 20, Tween® 40, Tween® 80, Tergitol® NP-40, LSS, TMAC and CHAPS. The rate of change was measured as the percent change in fluorescence of the test well as compared to the negative control in a concentration dependent manner.

Decreased change in fluorescent emission of the dye in the absence of a nucleic acid analog/target polynucleotide hybrid compared to the dye alone was not observed when the concentration of detergent was increased to 5.0%, however. FIG. 2 depicts the addition of detergents at a concentration of 5.0% to the reaction buffer. Detergents added to the reaction buffer included the nonionic detergents Tween® 20 (♦), Tween® 40 (⋄), Tween® 80 (∘), and Tergitol® NP-40 (); the anionic detergent LSS (); the cationic detergent TMAC (□); and the zwitterionic detergent CHAPS (×). Increasing the detergent to 5.0% in the reaction buffers resulted in a smaller percent change in the fluorescent rate for all reactions except those that included detergents LSS and CHAPS.

### Example 2

This example illustrates the usefulness of adding surfactants to the reaction buffer, which was sufficient for preparing samples for testing.

The addition of certain surfactants to the reaction buffer was found to permeabilize and/or lyse bacterial cells. Suprisingly, the assay for determining whether a particular target polynucleotide was present did not require further purification of the so-permeabilized/lysed cells.

Reaction buffer with different surfactants was used as permeabilization/hybridization buffer for bacteria to test whether separate nucleic acid isolation steps were necessary to perform the diagnostic method of the present invention. The addition of 0.5% Tween® 20, Tween® 40, Tergitol® NP-40, N-lauryl sarcosine sodium salt (LSS), or CHAPS to the phosphate reaction buffer effectively permeabilized and/or lysed bacteria from cultures grown overnight in tryptic soy broth, as demonstrated in the following experiment.

300 μl of overnight E. coli bacterial culture was centrifuged using standard procedures, resulting in a pellet of bacteria at the bottom of the tube. The supernatant was removed by aspiration. The pellet was then re-suspended in 390 μl of reaction buffer (5 mM phosphate) or lysis buffer (5 mM phosphate, pH 5.5, 0.05% surfactant) and incubated at room temperature for 10 minutes before an aliquot was used in the diagnostic reaction. 5 μl of the cells as resuspended in the two buffers were respectively and separately combined with 10 pmoles of 16S ncPNA (5′-ACT GCT GCC TCC CGT AG-3′ [SEQ ID NO:8] or 5′-TGC CTC CCG TAG-3′ [SEQ ID NO:9]) and 4 nmoles of 3,3′-diethylthiacarbocyanine iodide (DTCC dye) in a 50 μl total reaction volume. In all cases tested, the reaction buffer without surfactant showed a reaction indicating the presence of a target polypeptide, but it was slower and of a lesser extent of reaction than were those samples subjected to the lysis buffer. Accordingly, further testing focused on use of the lysis buffer.

The data generated in these studies were used to create the graph of FIG. 3. All reactions were conducted in reaction buffer with one of the surfactants at a concentration of 0.5%, which surfactants were: Tween® 20 (Δ), 0.5% Tween® 40 (⋄), 0.5% Tergitol® NP-40 (□), 0.05% lauryl sarcosine salt (∘) or 0.05% CHAPS (*) in phosphate buffer. The positive control (♦) used purified bacterial DNA in standard phosphate buffer; and the negative control (×) included all elements of the standard reaction apart from a target polynucleotide. A phosphate buffer control (▪) was included as well.

For each surfactant, the test well contained non-chiral PNA, target nucleic acid, 3,3′-diethylthiacarbocyanine iodide dye and lysis buffer (with surfactant). The test well was compared to the negative control that contained non-chiral PNA, 3,3′-diethylthiacarbocyanine iodide dye and lysis buffer (with surfactant). The phosphate buffer only test (▪) contained non-chiral PNA, target polynucleotide, 3,3′-diethylthiacarbocyanine iodide dye and reaction buffer (without surfactant). The phosphate buffer only well was compared to the negative control phosphate buffer that contained non-chiral PNA, 3,3′-diethylthiacarbocyanine iodide dye and reaction buffer (without surfactant).

An initial fluorescence reading was taken at time zero in the Tecan Genios microplate reader with the wavelengths set at 535 nm excitation and 590 nm emission. The mixtures were then exposed to a light stimulus using the Aurora 50/50 for 1 minute intervals with fluorescence reading being taken after each exposure for 10 minutes. For each reaction, the fluorescent emission values from the test well were compared to the negative control well and converted to the percent changed. The percent difference between the measured rate in samples containing the target nucleic acid and the rate in which the amount of target nucleic acid was zero indicated the presence of the target nucleic acid sequence. The difference corresponds to a relative decrease in fluorescence intensity of the test sample.

The presence of a target polynucleotide was detected in the presence of cell lysate, without requiring additional purification.

FIG. 3 shows the percent change in fluorescence compared to the lysis/hybridization buffer containing only phosphate buffer. The presence of the target nucleic acid sequence was determined for surfactants at 0.05% and 0.5% concentration. When light stimulus was applied, the rate of change in the fluorescence compared to the control corresponded to the presence of the target polynucleotide. The percent change in fluorescence was a decrease in the fluorescence intensity of the dye.

### Example 3

This example illustrates the effect of using different nucleic acid analogs in the diagnostic test of the present invention.

Different nucleic acid analogs were used to determine the presence or quantity of nucleic acid in a sample. Chiral PNA molecules, LNA molecules, and morpholino nucleic acid analogs were compared with non-chiral PNA molecules.

The non-chiral PNA and the chiral PNA had the sequence 5′ TGC CTC CCG TAG 3′ [SEQ ID NO:9], where the phosphodiester bonded sugar backbone of the native polynucleotide were replaced with a peptide bonded polypeptide backbone, as described further herein and well known within the art. Three LNA molecules designated LNA1, LNA2, and LNA3 were used, each having the same sequence of bases described here as SEQ ID NO:9, where one or more of the included nucleotides included a methylene bridge on their respective ribofuranose rings (forming a “locked” residue), as indicated: (1) LNA1 includes only locked residues; (2) LNA2 and (3) LNA3 include a subset of locked residues identified by the upper case letters at certain places on the sequence, as follows: 5′ TgC cTc CcG tAg 3′ for LNA2 and 5′ tGc cTc cCg tAg 3′ for LNA3. Morpholino nucleic acid analogs used here also had the base sequence of SEQ ID NO:9, formed from the analog nucleotides.

10 pmoles of nucleic acid analog, 10 pmoles of target polynucleotide having the sequence 5′ CTA CGG GAG GCA 3′ [SEQ ID NO: 12], and 4 nmoles of 3,3′-diethylthiacarbocyanine iodide dye were placed in a 50 μl total reaction volume to form a mixture. A negative control containing nucleic acid analog, 3,3′-diethylthiacarbocyanine iodide dye, reaction buffer, and a known (zero) amount of target polynucleotide was also tested. All the nucleic acid analogs and target polynucleotides were reconstituted in DNase- and RNase-free water to 100 μM stock and further diluted to 2 μM working stocks. The dye was dissolved in DMSO to generate an 8 mM stock. The 8 mM dye stock was further diluted to generate a 2 mM working stock in 5 mM phosphate buffer (pH 5.5). The reaction buffer was a 5 mM phosphate buffer (pH 5.5).

An initial fluorescence reading was taken at time zero in the Tecan Genios microplate reader with the wavelengths set at 535 nm for excitation and 590 nm for emission. The mixtures were then exposed to a 2000 foot-candle light stimulus using the Aurora 50/50 for 1 minute intervals and measuring the fluorescence at one minute intervals for 5 minutes. For each reaction, the fluorescent emission values from the test well (nucleic acid analog, target polynucleotide, dye, and reaction buffer) was compared to the negative control well (nucleic acid analog, dye and reaction buffer). The change in fluorescence was converted to the percent changed and normalized to the negative control containing only dye. The percent difference between the measured rate in samples containing the target nucleic acid and the rate in which the amount of target nucleic acid was zero indicated the presence of the target nucleic acid sequence.

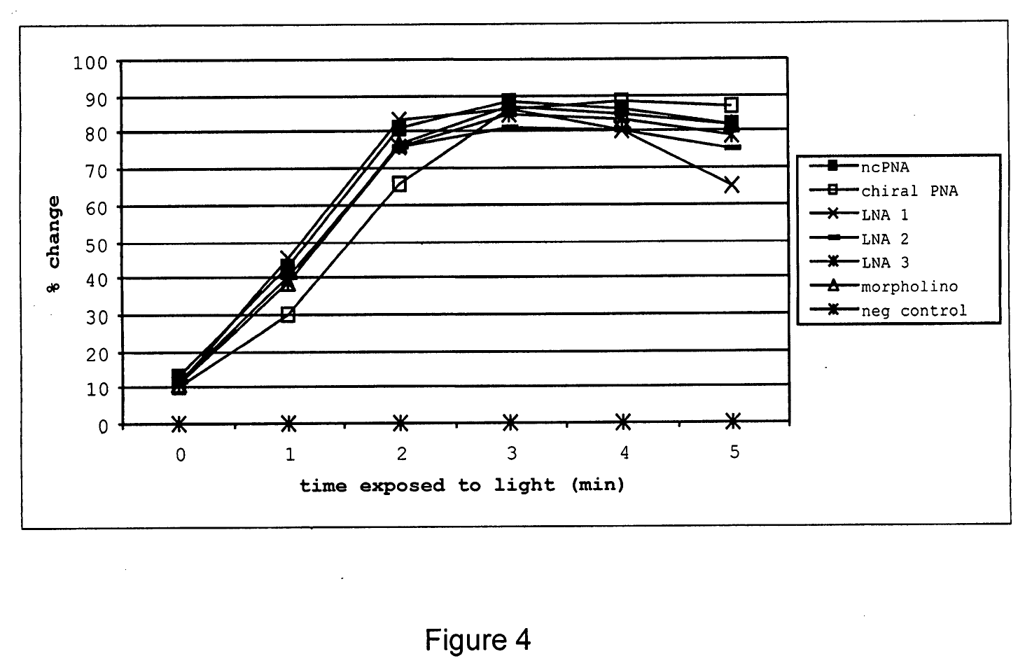

FIG. 4 displays a graph that is based on the data collected in the study of this example and manipulated as indicated above. As can be seen in the graph, the percent change in fluorescence intensity of each nucleic acid analog tested follows approximately the same profile of reaching in excess of 80% reduction in fluorescence emission by three minutes exposure to the light stimulus. Mixtures including non-chiral PNA (▪), chiral PNA (□), LNA 1, 2, and 3 (respectively represented by ×, , *), and morpholino (Δ) nucleic acid analogs showed a difference in the rate of change in an optical property of the dye compared to when no nucleic acid analog was present in the mixture (i.e., the negative control (the * indicating 0% change)). Of the various nucleic acid analogs tested, the chiral PNA may require more activation exposure in view of the percent change differences between mixtures including the chiral PNA and all others: after one and two minutes of light stimulus exposure, the cPNA mixture evidenced 30% and 65% changes in fluorescence, respectively; in contrast, the other reaction mixtures containing any of the other nucleic acid analogs evidenced at least 38% and 75% changes, respectively. Nonetheless, the data presented here supports the usefulness of all nucleic acid analogs tested for inclusion in the diagnostic method of the present invention.

### Example 4

This example illustrates different approaches for identifying dyes that are usefully employed in the context of the diagnostic method of the present invention.

In particular, this example presents data directed at determining if selected dyes exhibit a photo-induced change in fluorescence or absorbance based on results observed from light stimulus activation of the combinations of the respective dyes and: (1) a nucleic acid analog, namely a non-chiral PNA probe [SEQ ID NO: 1], or (2) a target oligonucleotide (“oligo”) that is exactly complementary to the PNA probe; or (3) the hybridized combination of the two. Each of these results were compared to a negative control mixture lacking ncPNA and oligo.

All dyes tested were from Sigma (St. Louis, Mo.) except the following: 3,3′-Diethylthiacyanine ethylsulfate (Organica), 3-Ethyl-9-methyl-3′-(3-sulfatobutyl)thiacarbocyanine betaine (Organica), 3-Carboxymethyl-3′,9-diethyl-5,5′-dimethylthiacarbocyanine betaine (Organica), 3,3′-Diallylthiacarbocyanine Bromide (Pfaltz and Bauer), 3,3′-Diethyl-2,2′-Oxathiacarbocyanine Iodide (Pfaltz and Bauer), [5-[2-(3-Ethyl-3H-benzothiazol-2-ylidene)-ethylidene]4-oxo-2-thioxo-thiazolidin-3-yl]-acetic acid (FEW), 1-Butyl-2-[3-(1-butyl-1H-benzo[cd]indol-2-ylidene)-propenyl]-benzo[cd]indolium tetrafluoroborate (FEW), 5,6-Dichloro-2-[3-(5,6-dichloro-1,3-diethyl-1,3-dihydro-benzimidazol-2-ylidene)-propenyl]-1,3-diethyl-3H-benzimidazolium iodide, and d) 1,3,3-Trimethyl-2-(2-[2-phenylsulfanyl-3-[2-(1,3,3-trimethyl-1,3-dihydro-indol-2-ylidene)-ethylidene]-cyclohex-1-enyl]-vinyl)-3H-indolium chloride (FEW).

Xenon Protocol: In one test protocol, a xenon light source was used to photoactivate reaction mixtures over a one minute period. Solutions containing concentrations of dye at 6 μM were prepared by diluting a stock solution of 5 mM dye (in methanol) in buffer (5 mM PO₄) plus surfactant (0.05% Tween® 80). The dyes used to prepare the 6 μM dye solutions are set forth in the summary of dyes studied that appears below in Table 2.

For the Xenon protocol, a 48 μL aliquot of each 6 μM dye solution was added to four wells of a 384-well microtiter plate (Costar, #3705). For each dye: a 1 μL aliquot of 5 μM oligo [SEQ ID NO:20] was added to the first and second well, a 1 μL aliquot of 5 μM ncPNA [SEQ ID NO:1] was added to the first and third well, a 1 μL aliquot of ddH₂O (Nanopure) was added to the second and third well, and a 2 μL aliquot of ddH₂O was added to the fourth well.

Fluorescent spectra at T₀ were obtained for each well using a Tecan Safire2 microplate reader. Parameters for the spectral scanning were: excitation range of 300 to 652 nm with a resolution (step size) of 11 nm, emission range of 375 to 723 nm with a resolution of 6 nm. The plate was then exposed to a 450W Xenon arc lamp (Ushio, #UXL-451-O) for 1 minute of photoactivation and fluorescent spectra were then read as before. Under the conditions used, dyes that showed a difference in an optical property of test reaction mixtures (containing ncPNA/target oligo) compared to the same optical property of dye-only reaction mixtures are indicated by a “yes” in Table 2. Distinctions between types of differences are not distinguished in table 2.

Aurora Protocol: In a second protocol, the reaction mixtures were exposed to the Aurora 50/50 light activation source and observed from 0 to 30 minutes. Solutions containing concentrations of dye at 25 μM were prepared by diluting stock solutions of 5 mM dye (in methanol) in molecular biology grade water (Hyclone, catalog #SH30538.03). The dyes used to prepare the 25 μM dye solutions are set forth in the summary of dyes studied that appears in Table 2.

For the Aurora Protocol, two different reaction mixtures were prepared for each dye. The first mixture consisted of 1 mL of 25 μM dye in water. The second mixture was identical to the first with the exception that 1 μL of a 50 μM ncPNA [SEQ ID NO:1]/target oligo [SEQ ID NO:20] mixture was added to the dye mixture. Spectral scans of the reaction mixtures were done using a 1 mL quartz cuvette (against water in a second reference cuvette) in a Shimadzu 160 UV Spectrophotometer. A spectrum from 200 nm to 800 nm for each reaction mixture was taken prior to exposure to a light stimulus (a 15 watt Aurora 50/50 fluorescent bulb, Fritz Industries). Parafilm was wrapped around the top of the cuvettes to prevent spillage of the solutions when the cuvettes were placed horizontally (length-wise) across the fluorescent light bulb. The solutions in the cuvettes were exposed to light for 5 minutes, followed by a spectral scan. This exposure-spectral scan cycle was repeated out to at least 10 minutes total light exposure. For each dye tested, the absorbance at lambda max (for time zero) of each spectrum was plotted as a function of time to determine the rate of absorbance decay for the ncPNA/target oligo mixture relative to the rate of absorbance decay for the dye only mixture. Under the conditions used, dyes that showed a difference in an optical property of test reaction mixtures (containing ncPNA/target oligo) compared to the same optical property of dye only reaction mixtures are indicated by a “yes” in Table 2. Distinctions between types of differences are not distinguished in table 2.

LED Protocol. In embodiment C, solutions containing various concentrations of dye were prepared by diluting stock solutions of 5 mM dye (in methanol or DMSO) in buffer (10 mM TE) with surfactant (0.1% Tween® 80) to a final concentration which provided an absorbance at lambda-max of 0.5-1.0 absorbance units (in a 50 μL volume in a 384-well white/clear microplate [NUNC, #242763]. The dyes used to prepare the dye solutions are set forth in the summary of dyes studied that appears in Table 2.

In this embodiment, four different reaction mixtures were prepared for each dye. The test reaction mixture was prepared by adding 30 μL dye solution, 10 μL of 500 nM ncPNA (in water) [SEQ ID NO:1] and 10 μL of 500 nM target oligo (in water) [SEQ ID NO:20] to a first well. The target oligo control reaction mixture was prepared by adding 30 μL dye solution, 10 μL molecular biology grade water (Hyclone) and 10 μL 500 nM target oligo [SEQ ID NO:20] to a second well. The ncPNA (probe) control reaction mixture was prepared by adding 30 μL dye solution, 10 μL of 500 nM ncPNA [SEQ ID NO:1] and 10 μL molecular biology grade water to a third well. The dye only control reaction mixture was prepared by adding 30 μL dye solution and 20 μL molecular biology grade water to a fourth well. Using a Safire2 microplate reader, absorbance measurements were taken at the lambda max of each dye tested. The plate was then removed from the reader and exposed to light from various banks of LEDs at various peak wavelengths. After 10 minutes photoactivation, the plate was removed from the light source and absorbance measurements were taken as previously. The plate was then removed from the reader, exposed to light from the photoactivator for an additional 50 minutes, and final absorbance measurements were taken. The absorbance at lambda max (for time zero, for each dye tested) was plotted against time to determine the rate of absorbance decay for the test reaction mixture, for the target oligo control reaction mixture, for the ncPNA control reaction mixture, and for the dye only control reaction mixture. Under the conditions used, dyes which showed a difference in an optical property of test reaction mixtures (containing ncPNA/target oligo) compared to the same optical property of dye only reaction mixtures are indicated by a “yes” in Table 2. Distinctions between types of differences are not distinguished in Table 2.

TABLE 2

| | | Xenon | Aurora | LED |
|---|---|---|---|---|
| # | Dye | Protocol | Protocol | Protocol |
| 1 | 3,3′-Dimethyloxacarbocyanine iodide | Yes | No | No |
| 2 | 3,3′-Diethylthiacyanine iodide | Yes | No | No |
| 3 | 3,3′-Diethylthiacyanine ethylsulfate | ND | No | No |
| 4 | 3,3′-Diethylthiacarbocyanine iodide | Yes | Yes | Yes |
| 5 | 3,3′-Diethyl-9-methylthiacarbocyanine | Yes | Yes | Yes |
| | iodide | | | |
| 6 | 3-Ethyl-9-methyl-3′-(3-sulfatobutyl) | ND | No | No |
| | thiacarbocyanine betaine | | | |
| 7 | 3-Carboxymethyl-3′,9-diethyl-5,5′- | ND | No | No |
| | dimethylthiacarbocyanine betaine | | | |
| 8 | 3,3′-Diethylthiadicarbocyanine iodide | Yes | No | Yes |
| 9 | 3,3′-Diethylthiatricarbocyanine iodide | Yes | No | No |
| 10 | 3,3′-Diethylthiatricarbocyanine | Yes | No | No |
| | perchlorate | | | |
| 11 | 3,3′-Diethyloxacarbocyanine iodide | Yes | No | No |
| 12 | 3,3′-Diethyloxadicarbocyanine iodide | Yes | Yes | No |
| 13 | 3,3′-Dipropylthiacarbocyanine iodide | Yes | Yes | Yes |
| 14 | 3,3′-Dipropylthiadicarbocyanine | No | No | No |
| | iodide | | | |
| 15 | 3,3′-Dipropyloxacarbocyanine iodide | No | No | No |
| 16 | 3,3′-Dibutylthiacarbocyanine iodide | Yes | Yes | Yes |
| 17 | 3,3′-Dipentylthiacarbocyanine iodide | Yes | Yes | Yes |
| 18 | 3,3′-Dihexyloxacarbocyanine iodide | No | No | No |
| 19 | 3,3′-Diallylthiacarbocyanine Bromide | Yes | Yes | Yes |
| 20 | 3,3′-Diethyl-2,2′-Oxathiacarbocyanine | Yes | No | No |
| | Iodide | | | |
| 21 | 1,1′-Diethyl-2,2′-cyanine iodide | Yes | No | No |
| 22 | 1-1′-Diethyl-2,2′-carbocyanine iodide | Yes | Yes | Yes |
| 23 | 1,1′-Diethyl-2,2′-carbocyanine | ND | Yes | Yes |
| | bromide | | | |
| 24 | 1,1′-Diethyl-4,4′-carbocyanine iodide | No | No | No |
| 25 | 1,1′-Diethyl-3,3,3′,3′- | No | No | No |
| | tetramethylindocarbocyanine iodide | | | |
| 26 | 1,1′-Dipropyl-3,3,3′,3′- | No | No | No |
| | tetramethylindocarbocyanine iodine | | | |
| 27 | [5-[2-(3-Ethyl-3H-benzothiazol-2- | ND | No | No |
| | ylidene)-ethylidene]-4-oxo-2-thioxo- | | | |
| | thiazolidin-3-yl]-acetic acid | | | |
| 28 | 1-Butyl-2-[3-(1-butyl-1H- | ND | No | No |
| | benzo[cd]indol-2-ylidene)-propenyl]- | | | |
| | benzo[cd]indolium tetrafluoroborate | | | |
| 29 | 5,6-Dichloro-2-[3-(5,6-dichloro-1,3- | ND | No | ND |
| | diethyl-1,3-dihydro-benzimidazol-2- | | | |
| | ylidene)-propenyl]-1,3-diethyl-3H- | | | |
| | benzimidazolium iodide | | | |
| 30 | 1,3,3-Trimethyl-2-(2-[2- | ND | No | No |
| | phenylsulfanyl-3-[2-(1,3,3-trimethyl- | | | |
| | 1,3-dihydro-indol-2-ylidene)- | | | |
| | ethylidene]-cyclohex-1-enyl]-vinyl)- | | | |
| | 3H-indolium chloride | | | |
| 31 | 4,5,4′,5′-Dibenzo-3,3′-diethyl-9- | ND | No | No |
| | methyl-thiacarbocyanine bromide | | | |

Of 31 dyes tested using the three different protocols set forth above, only six were shown to not change color; another eight have not demonstrated positive results, but were not tested with all three protocols as yet and therefore remain inconclusive. Over half of the total number of dyes tested displayed positive results. Accordingly, placing the eight that are as yet incompletely tested to the side, the results are currently indicating that 17 out of 23 fully tested dyes show positive results, i.e., a 74% rate of success.

### Example 5

This example illustrates a test designed to immobilize the nucleic acid analog on a solid substrate such that target polynucleotides from a cell lysate can be detected.

Transgenic and non-transgenic leaf tissue samples were collected. Two punches from each maize leaf were taken and placed in a 0.2 ml PCR tube. 100 μl of lysis/hybridization buffer (50 mM KCl, 10 mM Tris HCl, 0.1% Triton X) was added, and the samples were heated 5 minutes at 95° C. in a thermal cycler. The tubes were briefly centrifuged to pellet denatured materials; and the resultant supernatant was used on the crude clarified lysate. Tubes once centrifuged were put aside at room temperature until needed. Aliquots of 5 μl of crude-clarified lysate was added to each reaction, as noted below.

The wells in a streptavidin-coated microtiter plate were prepared by washing 3× with 200 μl of 1× PBST (phosphate buffered saline+0.05% Tween® 20).

Standard strip tubes (Perkin Elmer, Catalog #N801-0580) were used to mix reagents for the control and experimental reactions in accordance with the present diagnostic method, and marked accordingly, as follows: (1) immobilized test—transgenic lysate/ncPNA/dye; (2) immobilized test—wildtype lysate/ncPNA/dye; (3) positive control—oligo/ncPNA/dye; (4) negative control—ncPNA/dye; (5) negative control—transgenic lysate/dye; (6) negative control—oligo/dye; and (7) negative control—dye only. The 5 μl aliquots of crude-clarified maize lysate sample (either transgenic or wild-type) were added to the appropriate tubes that were intended to receive lysate sample material. A positive control sample included 5 μl of the 2 μM stock solution of the target polynucleotide sequence 5′ ACGCACAATCCCACTATC 3′ [SEQ ID NO:20]. Components were added to the various tubes as set forth in Table 4 below.

TABLE 4

| | (1) or (2) | | | | | |
|---|---|---|---|---|---|---|
| | Immobilized | | | | | |
| | samples with | | | (5) | | (7) |
| | cellular lysate, | | (4) | Negative | (6) | Negatie |
| | transgenic (1) | (3) | Negative | control: | Negative | control: |
| | or | Positive | control: | transgenic | control: | Dye |
| | wildtype (2) | control | ncPNA + dye | lysate + dye | Oligo + dye | only |
| ncPNA | 2.5 μl | 2.5 μl | 2.5 μl | 0 | 0 | 0 |
| lysate DNA | 5 μl | 0 | 0 | 5 μl | 0 | 0 |
| 5 mM PBST | 43.5 μl | 43.5 μl | 47.5 μl | 45 μl | 45 μl | 50 μl |
| Oligo control | 0 | 5 μl | 0 | 0 | 5 μl | 0 |

The nucleic acid analog used was an ncPNA that included a sequence that was specific for the 35S promoter found in many genetically-modified plants. The ncPNA has at least one biotin molecule attached at the ncPNA's 5′ end: 5′ Bio-ooooo-GATAGTGGGATTGTGCGT 3′ [SEQ ID NO:1]. The “ooooo” represents multiple residues of 8-amino-3,6-dioxaoctanoic acid which are hydrophilic flexible linker molecules that are inserted between the biotin and the ncPNA. Once the binding mixtures were completed in the strip tubes, 50 μl of each sample was transferred to its corresponding well on the microtiter plate. Samples were incubated covered for 30 minutes at room temperature, with gentle shaking. After the 30 minute incubations, the reaction mixes were respectively removed from the wells by . aspiration. Wells were washed three times with 200 μl of 1× PBST followed by three washes with 200 μl of 5 mM phosphate buffer with 0.05% NP-40 surfactant (“phosphate/NP-40 buffer”). A master dye mix was generated by combining 384 μl of phosphate/NP-40 buffer plus 16 μl of 2 mM dye (“buffer/dye”). A 50 [l aliquot of buffer/dye was added to each well. The plate was placed in the Tecan Genios microplate reader. The initial fluorescence was read without light exposure. Samples were exposed to the Aurora 50/50 fluorescent light and readings were taken after every 2 minutes of light exposure up to 26 minutes. Fluorescence measurements were converted to percent change (signal from test samples compared to signa from ncPNA/dye-only sample) and plotted as a function of time.

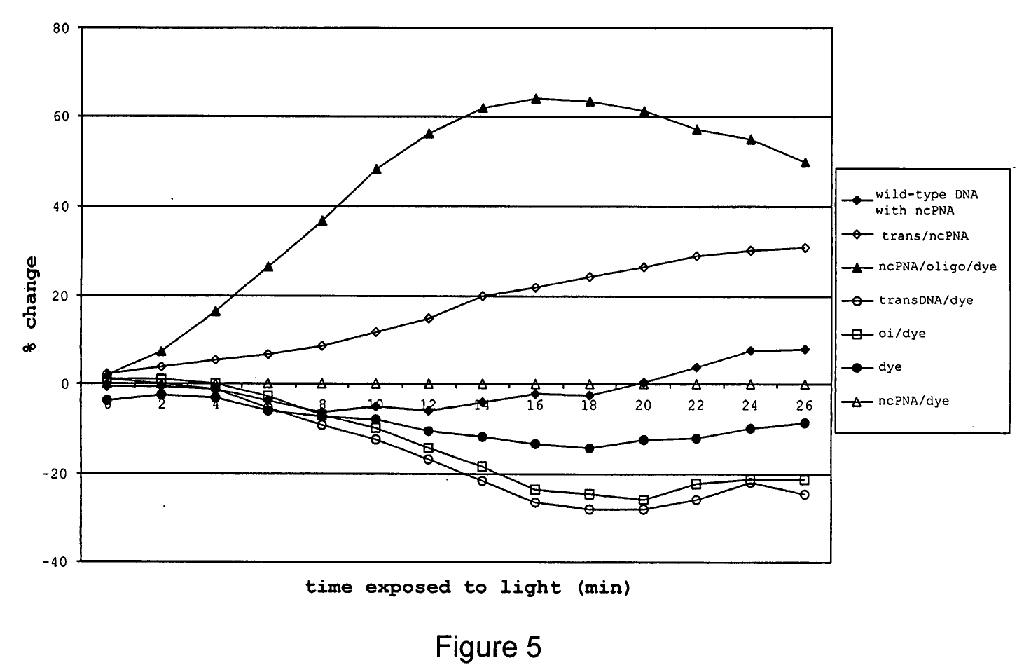

FIG. 5 shows the percent change in fluorescence signal over time for a reaction mixture where samples are immobilized on a streptavidin-coated plate. The positive control reaction occurs very rapidly (solid triangles). The transgenic leaf sample (open diamonds) shows a reduced fluorescence over time and a constant increase in percent change over time, while the wild type leaf sample (solid diamonds) shows a minor increase over time. Fluorescence signal for the reaction mixtures containing ncPNA/dye (open triangles), dye only (solid circles), transgenic DNA/dye without ncPNA (open circles) and target polynucleotide sequence/dye (open squares) samples showed no substantial change over time. Values in the negative indicate that the optical property of the reaction mixture changed at a slower rate than the ncPNA/dye reaction mixture.

Regarding the results of this Example 5, applicants indicate that repeating the study disclosed here was attempted, but without success. Unfortunately, time lapsed between the original studies and the attempt to repeat. In consequence, several of the materials used in the earlier work were not available to the later attempt. Among the material not available were the genomic DNA (new preps made using a different protocol), a different manufacturer of the PNA was used, different microplates and lot numbers were necessarily used as well, and nearly or all of the reagents were of different lots. Out of an abundance of caution to state facts surrounding our work causes us to include this information. We are to date of the filing of this application unaware of any reason why the original study could not be repeated, which is consistent with many, many other positive results reported here.

### Example 6

This example illustrates one embodiment of a gel-based assay using the diagnostic test of the present invention.

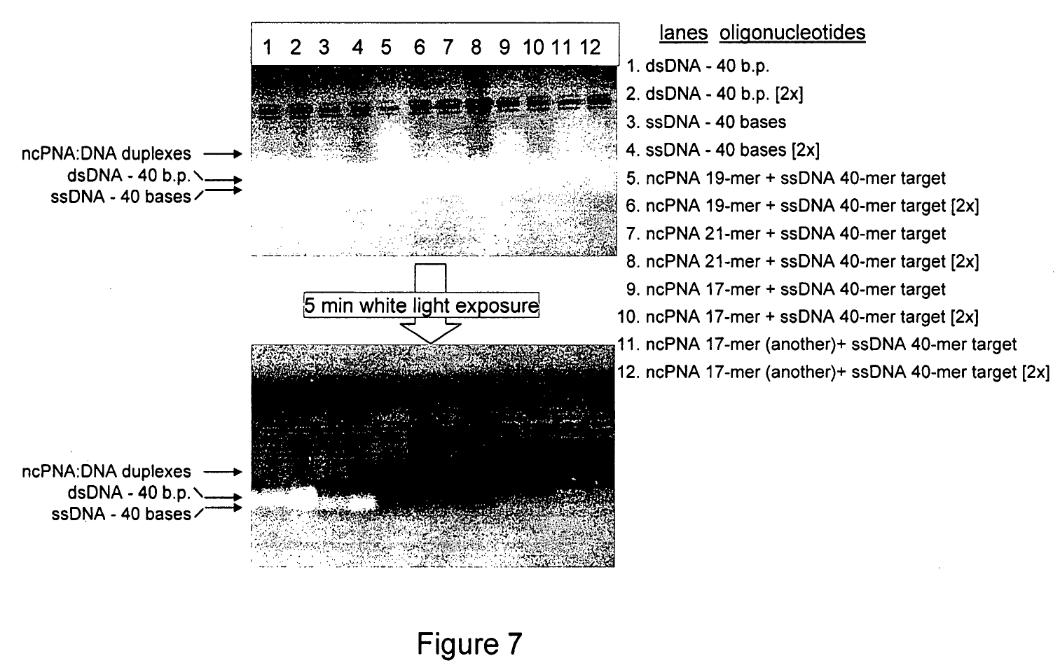

The methods presented herein can be used to identify the presence of a target polynucleotide in a gel-based assay. Complementary pairs of ncPNA and single-strand polynucleotides were mixed together for at least 30 minutes at room temperature and loaded onto a 3% agarose gel (HK—What buffer used in gel?). The agarose gel contained 2.5 uM of DTCC. As shown in FIG. 7, the first four lanes contained double- and single-stranded DNA size standards of 40 base pairs (or 40 bases) each, as follows:

[SEQ ID NO: 48]

| | |
|---|---|
| Lane 1 | 100 pmoles double-stranded DNA (5′- |
| | CCAGGACGACCGGGTCCTTTCTTGGATCAACCCGCTCAAT-3′, |
| | plus complementary strand |

[SEQ ID NO: 49])

| | |
|---|---|
| | 5′- |
| | ATTGAGCGGGTTGATCCAAGAAAGGACCCGGTCGTCCTGG-3′; |
| Lane 2 | 200 pmoles double-stranded DNA (same as |
| | above); |

[SEQ ID NO: 50]

| | |
|---|---|
| Lane 3 | 100 pmoles single-stranded DNA (5′- |
| | TGCTAGCCGAGTAGTGTTGGGTCGCGAAAGGCCTTGTGGT-3′; |
| and | |
| Lane 4 | 200 pmoles single-stranded DNA (same as |
| | above) |

The remaining lanes contained different combinations of ncPNA and its respective complementary single-stranded target polynucleotide sequences, as follows: Lane 5, 100 pmoles ncPNA 19-mer having the following sequence GTTGATCCAAGAAAGGACC-lysine [SEQ ID NO: 51] plus 100 pmoles of single-stranded DNA 40-mer target polynucleotide having the following sequence 5′-CCAGGACGACCGGGTCCTTTCTTGGATCAACCCGCTCAAT-3′ [SEQ ID NO: 48]; Lane 6, same as lane 5 except 200 pmoles of ncPNA 19-mer plus 200 pmoles of single-stranded DNA 40-mer target polynucleotide; Lane 7, 100 pmoles ncPNA 21-mer having the following sequence GTTGATCCAAGAAAGGACCCG-lysine [SEQ ID NO: 51] plus 100 pmoles single-stranded DNA 40-mer target polynucleotide having the following sequence 5′-CCAGGACGACCGGGTCCTTTCTTGGATCAACCCGCTCAAT-3′ [SEQ ID NO: 48]; Lane 8, same as lane 7 except 200 pmoles of ncPNA and 200 pmoles of single-stranded 40-mer DNA target; Lane 9, 100 pmoles ncPNA 17-mer having the following sequence TTTCGCGACCCAACACT-lysine [SEQ ID NO: 53] plus 100 pmoles of single-stranded DNA 40-mer target polynucleotide having the following sequence 5′-TGCTAGCCGAGTAGTGTTGGGTCGCGAAAGGCCTTGTGGT-3′ [SEQ ID NO: 50]; Lane 10, same as lane 9 except 200 pmoles ncPNA 17-mer and 200 pmoles single-stranded DNA 40-mer target polynucleotide; lane 11, 100 pmoles of another ncPNA 17-mer having the following sequence AGTGTTGGGTCGCGAAA-lysine [SEQ ID NO: 55] plus 100 pmoles of single-stranded DNA 40-mer target polynucleotide having the following sequence 5′-ACCACAAGGCCTTTCGCGACCCAACACTACTCGGCTAGCA-3′ [SEQ ID NO: 56].

Electrophoresis was conducted in the 3% agarose/2.5 uM DTCC/1× TBE gel at 200V for 20 minutes. After illuminating the gel with white light from the Aurora 50/50 for 5 minutes, the gel was observed. A 254 nm UV transilluminator and B&W Polaroid camera were used to photograph the gel before and after photoactivation (upper and lower panels, respectively). The gel generally had a pink color with a faint pink band in lanes 1-4 that contain ssDNA and dsDNA. The ncPNA/target polynucleotide hybrid was expected to migrate more slowly than either the single-stranded or double-stranded DNA 40 bp standards in lanes 1-4. Indeed, at the expected location was a region that lacked color after photoactivation. These “holes” show up as a darker area on the second panel of FIG. 7 identified the presence of the NAA/TP hybrid.

The method of identifying the target polynucleotides in gel-based systems according to the methods disclosed herein can be adapted to any gel-based method of identifying target polynucleotides, including Southern, Northern, and Northwestern blotting techniques. Additionally, other agarose gels containing the dye 3,3′-diallylthiacarbocyanine iodide dye demonstrated similar results. Other dyes are anticipated to work similarly

### Example 7

This example demonstrates detection of single nucleotide polymorphisms (SNPs) using two nucleic acid analog sequences.

An example of the strategy for detecting point, insertion, and deletion mutations can be found in FIGS. 6C and 6D. PNAs which only partially hybridize to target DNA (due to mutations) may form helical duplexes too short to participate in light-activated photobleaching of DiSC₂(3). The following experiment illustrates the affects of point mutations in the assay for the detection of SNPs.

The sequence of ncPNA1 was 5′ Bio-OOOOO-GATAGTGGGATTGTGCGT 3′ [SEQ ID NO:1]. The sequence of ncPNA2 was 5′ TCACATCAATCCACT-lys 3′ [SEQ ID NO:21]. The “O” represents the linker molecule 8-amino-3,6-dioxaoctanoic acid.

Table 5 lists the DNA oligonucleotide sequences with the mismatch bases in lower case. 100 μM single-stranded DNA stock solutions containing different single-stranded DNA 12-mers were prepared. Wild-type DNA oligonucleotide [considered to be the (−) strand] was fully complementary to six bases on ncPNA1 (3′ end) and to six bases on ncPNA2 (5′ end). Four other DNA oligonucleotides contained 1 or 2 or 4 point mutations (mu1, mu2, mu3, mu4) were mismatches with bases on either ncPNA1 or ncPNA2. Each of the 5 DNA oligonucleotides also had an exact complementary DNA oligonucleotide [considered to be the (+) strands].

To prepare the double-stranded complementary and mutated sequences, 1.2 μl of 100 uM of each (+) DNA strand was mixed with 1 μl of the 100 uM complementary (−) DNA strand and diluted to a final concentration of 2 μM. These solutions were heated to 95° C. for 5 minutes and allowed to cool to room temperature to promote annealing. The sequences of the complementary sequence and the mutated sequences (mu1-mu4) are set forth in Table 5.

TABLE 5

| | | |
|---|---|---|
| Name | + seq 5′ to 3′ | − seq 5′ to 3′ |

| | | |
|---|---|---|
| wild type | GTGCGTTCACAT | ATGTGAACGCAC |
| | [SEQ ID NO: 22] | [SEQ ID NO: 23] |
| mu1 | cTGCGTTCACAT | ATGTGAACGCAg |
| | [SEQ ID NO: 24] | [SEQ ID NO: 25] |
| mu2 | GTGCGTTCACAa | tTGTGAACGCAC |
| | [SEQ ID NO: 26] | [SEQ ID NO: 27] |
| mu3 | caGCGTTCACAT | ATGTGAACGCtg |
| | [SEQ ID NO: 28] | [SEQ ID NO: 29] |
| mu4 | caGCGTTCAGgt | acCTGAACGCtg |
| | [SEQ ID NO: 30] | [SEQ ID NO: 31] |

A ncPNA master mix was made by combining 105 μl of 2 μM ncPNA1, 105 μl of 2 μM ncPNA2, 693 μl of 5 mM phosphate buffer (pH 5.5) containing 0.05% Tween® 80, 14% methanol, and 42 μl of 0.75 mM 3,3′-diethylthiacarbocyanine iodide dye. This mixture contains final concentrations of 220 nM ncPNA1, 220 nM ncPNA2, and 33 uM 3,3′-diethylthiacarbocyanine iodide dye. For reactions that contained both ncPNAs and the dsDNA, 15 μl of each double-stranded polynucleotide and 135 μl of the ncPNA master mix was added to each tube. From each tube a 50 μl aliquot of each mix was transferred to three wells of the white microtiter plate (Greiner).

A control “DNA and dye” only tube that contained complementary DNA oligonucleotides and dye was prepared by combining 20 μl of annealed wild-type DNA, 8 μl of a 0.75 mM solution of 3,3′-diethylthiacarbocyanine iodide dye, and 172 μl of 5 mM phosphate buffer (pH 5.5) containing 0.05% Tween® 80 and 14% methanol. Three 50 μl aliquots of this mixture was dispensed into three wells. Two control “PNA and dye” tubes containing ncPNA1 or ncPNA2 were made by combining 20 μl of either ncPNA1 or ncPNA2, 8 μl of the 0.75 mM solution of 3,3′-diethylthiacarbocyanine iodide dye, and 172 μl of the 5 mM phosphate buffer (pH 5.5) containing 0.05% Tweeno 80 and 14% methanol. 50 μl of this mixture was aliquoted into three wells. The plate was placed in the Tecan Genios microplate reader and an initial fluorescence was read. Samples were exposed to the Aurora 50/50 fluorescent light and readings were taken after every one minute of light stimulus for five minutes.

As can be seen in the FIG. 8, fluorescence emission over a period of five minutes remained substantially stable at about 27000 to 32000 relative fluorescence units (RFUs) in sample mixtures lacking ncPNA1 or ncPNA2 or exactly complementary DNA relative to the nucleic acid analog sequence defined by both ncPNA1 and ncPNA2. After four minutes of light exposure, the fully complementary sequence can easily be differentiated (open diamonds) from the ncPNA only (1 or 2), DNA only and SNP DNA with both ncPNAs by the substantial reduction in fluorescence. Interestingly, the start of fluorescence emission reduction started in samples that included the SNP variances mu1 and mu2 at about four to five minutes of incubation. This observation is extended in the data illustrated in FIG. 9, when the fluorescent emission after light stimulation of the mixture containing a fully complementary sequence versus a mixture containing a mutated oligonucleotide with two or four mutations (i.e., one or two, 2 base-pair changes). Again, where one of the two nucleic acid analogs (ncPNA1 or ncPNA2) were absent, the fluorescence emission remained unwaveringly high at about 32000 relative fluorescence units. Lacking exactly complementary DNA with respect to the sequence of ncPNA1 and ncPNA2 provided the same result, essentially. Where both nucleic acid analog and exactly complementary DNA is included in the mixtures, fluorescent emissions dropped off dramatically at some point after the second minute of light exposure, and decidedly so between the third and fourth minute of exposure. The reactions using the same ncPNAs with variants having two or four mismatches, fluorescent emission could be seen to have started dropping off by the fourth minute of light exposure.

### Example 8

This example compares the two detergents, Tween®80 and Tergitol® NP-40, in a buffer containing 5 mM sodium phosphate (pH 5.5) in reactions with and without NAA/TP hybrids to demonstrate increased dye resistance to photoactivation.

In the following example, a identical mixtures were made in buffer containing either 0.05% Tergol® NP-40 (Sigma Catalog No. 127087) or 0.05% Tween® 80 (Sigma, Catalog No. P1754). The use of the white plate correlated with readings of an accelerated reaction rate

A 100 μM stock solution of ncPNA having the sequence 5′ Bio-OOOOO-GATAGTGGGATTGTGCGT 3′ [SEQ ID NO:1] was diluted to 2 μM in 5 mM sodium phosphate buffer (pH 5.5) with 0.05% Tween® 80 (“tw80 buffer”). An identical dilution was created using 5 mM sodium phosphate buffer (pH 5.5) with 0.05% NP-40 (NP40 buffer). In separate tubes, a 100 μM stock of complementary DNA with the sequence 5′ ACGCACAATCCCACTATC 3′ [SEQ ID NO:20] was diluted to a concentration of 2 μM using either the tw80 buffer or the NP40 buffer as above.

The mixture set forth in Table 6 was made.

TABLE 6

| Component | amount | multiplier | Total |
|---|---|---|---|
| ncPNA | 5 μl | ×13 | 65 μl |
| Oligo | 5 μl | ×13 | 65 μl |
| Buffer (tw80) or (NP40) | 38 μl | ×26 | 988 μl |
| Dye (0.75 mM) | 2 μl | ×26 | 56 μl |

The dye was first added to the buffer and mixed. For the NAA/TP reactions, 520 μl of buffer/dye mixture, 65 μl of ncPNA, and 65 μl of oligo were added and mixed. For the “Dye Only” control, 520 μl was transferred to a fresh tube and an additional 130 μl of buffer (either with Tw80 or NP40) was added. Aliquots of 50 μl were dispensed into 12 wells (for each detergent buffer and the “Dye Only” control) of a white Greiner 96-well microtiter plate (Catalog No. 655088).

The plate was then placed in a Tecan Genios microplate reader and an initial fluorescence was measured. The plate was exposed to the Aurora 50/50 photoactivator and readings were taken after every 1 minute of light exposure up to 7 minutes of total exposure.

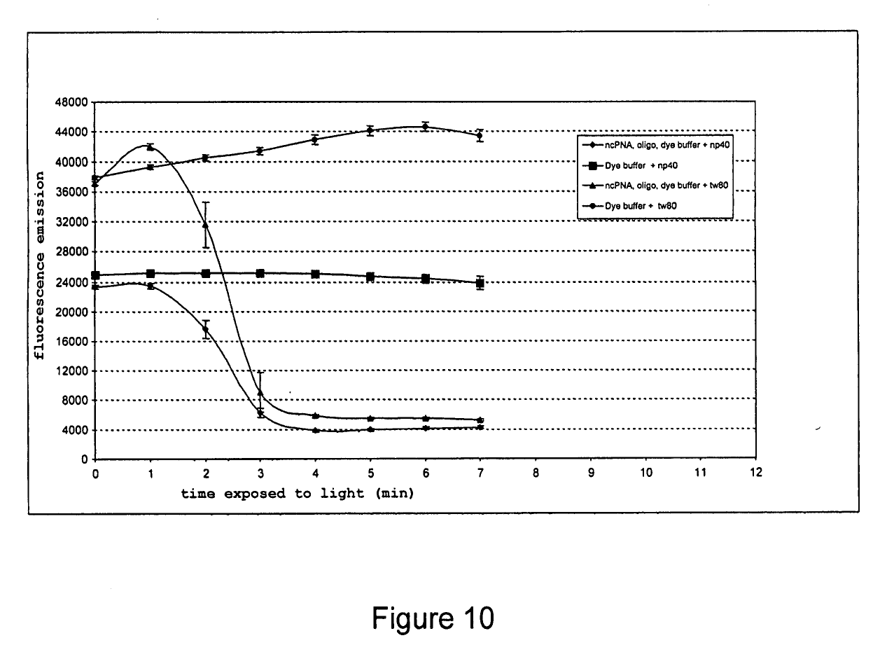

The fluorescence at various times (of total exposure to the Aurora 50/50 light) is presented graphically in FIG. 10. The lines on the graph represented data from the identified reactions: Solid circle, the control of dye only in tw80 buffer; solid triangles, the ncPNA/oligo reaction in tw80 buffer; the solid squares, the control of dye only in NP40 buffer; and solid diamonds, the ncPNA/oligo reaction in NP40 buffer. As clearly indicated on the graph, the two controls of dye only demonstrate that the fluorescent emissions remained constant over the observed seven minutes of light exposure. Additionally, dye in solution with Tween® 80 had and maintained fluorescent emission at approximately a 50% greater level than that of the dye in solution with NP-40. Both reactions demonstrated observable responses by the second minute after light exposure began, and dramatically so by the third minute after the start of light exposure.

Accordingly, this example demonstrates two aspects of the present invention: (1) use of surfactants NP-40 and Tween® 80, both non-ionic, correlated with increased dye fluorescence stability upon photoactivation, and (2) Tween® 80 correlates with about a 50% increased level of fluorescent emission as compared to reactions with NP-40.

### Example 9

This example illustrates the effect that different plates have regarding the profiles of the reactions of the amino acid analog, target polynucleotide, and dye, in accordance with the present invention.

The microliter plates listed in Table 7 were obtained and tested to determine whether different color schemes of such materials have an effect on the reaction of a nucleic acid analog, a complementary target polynucleotide, and a dye, as provided by the present invention.

TABLE 7

| | Plate Brands | Description | Color Scheme |
|---|---|---|---|
| 1. | Greiner 655073 | 96-well | white with white bottom |
| 2. | Greiner 655088 | 96-well, μclear | black with clear bottom |
| 3. | Greiner 355892 | 96-well, glass bottom | black with clear bottom |
| 4. | Greiner 655095 | 96-well, μclear | white with clear bottom |
| 5. | Greiner 655096 | 96-well, μclear | black with clear bottom |
| 6. | Greiner 781091 | 384-well | black with clear bottom |
| 7. | Greiner 788096 | 384-well, small volume | black with clear bottom |
| 8. | Greiner 788092 | 384-well, small volume | black with clear bottom |
| 9. | Greiner 781892 | 384-well, glass bottom | black with clear bottom |
| 10. | NUNC 436014 | 96-well, streptavidin | clear with clear bottom |
| 11. | NUNC 265302 | 96-well | white with white bottom |
| 12. | NUNC 237105 | 96-well | black with black bottom |
| 13. | NUNC 265301 | 96-well, optical bottom | black with clear bottom |
| 14. | NUNC 265301 | 96-well, optical bottom | white with clear bottom |
| 15. | Costar 3601 | 96-well, high binding | black with clear bottom |
| 16. | Corning 3651 | 96-well | black with clear bottom |
| 17. | Costar 3631 | 96-well | black with clear bottom |
| 18. | Costar 3615 | 96-well, special optics | black with clear bottom |
| 19. | Costar 3632 | 96-well | white with clear bottom |
| 20. | Costar 3693 | 96-well, ½ area | white with white bottom |
| 21. | BD | 96-well, streptavidin | black with black bottom |
| | Biosciences | | |
| | 353241 | | |
| 22. | BD | 96 well | white with white bottom |
| | Biosciences | | |
| | 354742 | | |
| 23. | Matriplate | 384-well, glass bottom | black with clear bottom |

Each plate listed in Table 7 was tested. In a 50 μL test reaction, an 18-mer ncPNA with the sequence 5′ GATAGTGGGATTGTGCGT 3′ [SEQ ID NO:1] (N-terminus to C-terminus) and a complementary DNA oligonucleotide target (final concentration 200 nM for both) were mixed with 3,3′-diethylthiacarbocyanine iodide dye (final concentration 300 μM) in a phosphate buffer with NP-40. A 50 μL control reaction mixture of phosphate buffer with NP-40 with 3,3′-diethylthiacarbocyanine iodide dye (final concentration) was run simultaneously. Light was applied to the plates, and the fluorescence intensity over time was measured.

In plates with clear bottoms, light was projected upwards through the well. The distance between the light source and the bottom of the well was approximately ¼ inch. In plates with black or white bottoms, light was projected downwards into the well at a distance of about 1 inch from the surface of the liquid. A fluorescent reading prior to light exposure was taken followed by subsequent readings every 60 seconds during exposure to light. Data were plotted as fluorescence vs time with each data point representing at least 12 identical reactions, plus and minus the standard deviation.

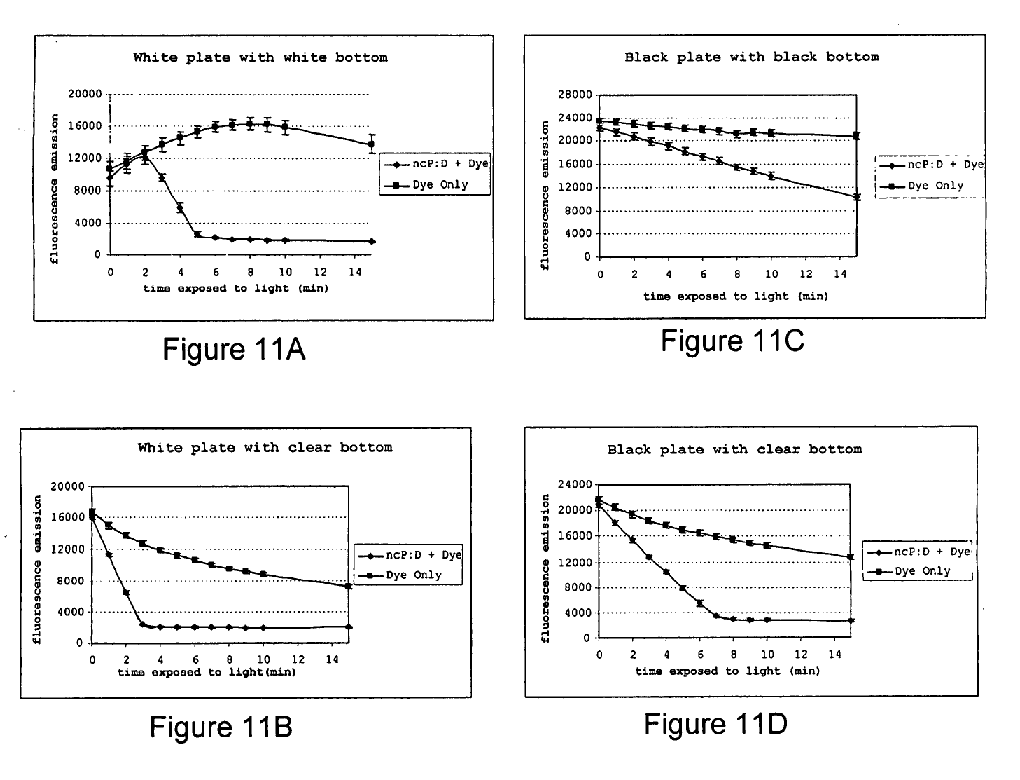

FIGS. 11A-D present the data graphically of replicate test reactions and controls reactions which were run in a white plate with white bottom (FIG. 11A); a white plate with clean bottom (FIG. 11B); a black bottom (FIG. 11C); and a black plate with clear bottom (FIG. 11D). This experiment demonstrates a considerable effect of the reflective (i.e., white) or absorptive (i.e., black) nature of the plate on the rate of fluorescence decay of 3,3′-diethylthiacarbocyanine iodide dye.

When the light source projected downward into the reaction (FIGS. 11A and 11B), there was an accelerated rate of fluorescence decay of the test reaction while the control reaction demonstrated minimal fluorescence decay. When a white plate with white bottom was used, the fluorescence of the “Dye Only” reaction increases from time zero to 15 minutes (FIG. 11B). In a black plate with a black bottom the rate of fluorescence decay of the test reaction was significantly slower than the same reaction in a white plate with the white bottom.

In plates containing clear bottoms (where the light was projected upwards through the wells), the white plates demonstrated a faster rate of fluorescence decay of the test reactions compared to the black plates (FIGS. 11C and 11D), reaching the minimum observed value in about three minutes versus about seven minutes. The control reactions also demonstrated a slightly faster rate of fluorescence decay in the white plates compared to the black plates.

In general, the results were not significantly different using different brands (i.e., NUNC, Greiner, or Costar) of microplates. It was observed, however, that the black plates correlated with nearly a 50% increase in fluorescence emission relative to the white plates (see the 0 time fluorescence level for each set of experiments, for example). It was also observed that the white plates correlated with an test reaction that was faster in reaching the minimum fluorescence level.

### Example 10

This example describes an experiment and results thereof that tested the use of a modified reaction buffer that included surfactant and alcohol.

A modified phosphate buffer was identified that provides greatly enhanced stabilization of dye and reduced background signal. The modified buffer consisted of the 5 mM phosphate buffer described above, plus 0.05% Tween 80 and 14% methanol. Stabilization of the dye was also enhanced by using less light. The reactions described in this example were conducted in a white 96 well flat white bottom microtiter plate (Greiner).

Reactions were set up in triplicate and loaded on the microtiter plate. Reaction mixes were made from 7.5 mM 3,3′-diethylthiacarbocyanine iodide dye stock, 100 μM ncPNA stock, Bio 18 (5′ Bio-ooooo-GATAGTGGGATTGTGCGT 3′ [SEQ ID NO:1]), 100 μM complementary DNA set (5′ GATAGTGGGATTGTGCGT 3′ [SEQ ID NO:1]; 5′ ACGCACAATCCCACTATC 3′ [SEQ ID NO:20]), 1 μl of each homologous DNA pair was mixed equally in annealing buffer (Sambrook, et al) at a concentration of 2 μM and heated to 95° C. for five minutes, then allowed to cool to room temperature. The ncPNA were diluted to 2 μM working stock.

In a PCR strip tube, 20 μl of 2 μM ncPNA and 20 μl of complementary DNA was mixed, making a test reaction mixture. 20 μl of ncPNA and 20 of buffer was added to a well making a ncPNA/dye control reaction mixture. 20 μl of DNA and 20 μl of buffer were added to a well making a DNA/dye control reaction mixture, and 40 μl of buffer was added to the dye only control reaction mixture. A master dye buffer mix was made by adding 36 μl of 0.75 mM dye to 684 μl of 5 mM PO₄ buffer plus 0.05% Tween® 80 with 14% methanol. The solution was mixed and 160 μl was dispensed to each of the 4 wells of the PCR strip tube. The solutions were mixed and 50 μl of each reaction mix was dispensed into 3 wells (triplicate) of the white plate. The plate was placed in the Tecan Genios microplate reader and an initial fluorescence measurement was taken (before photoactivation). Samples were exposed to the Aurora 50/50 fluorescent light at half its light intensity and fluorescence measurements were taken after every 1 minute of light exposure for 10 minutes.

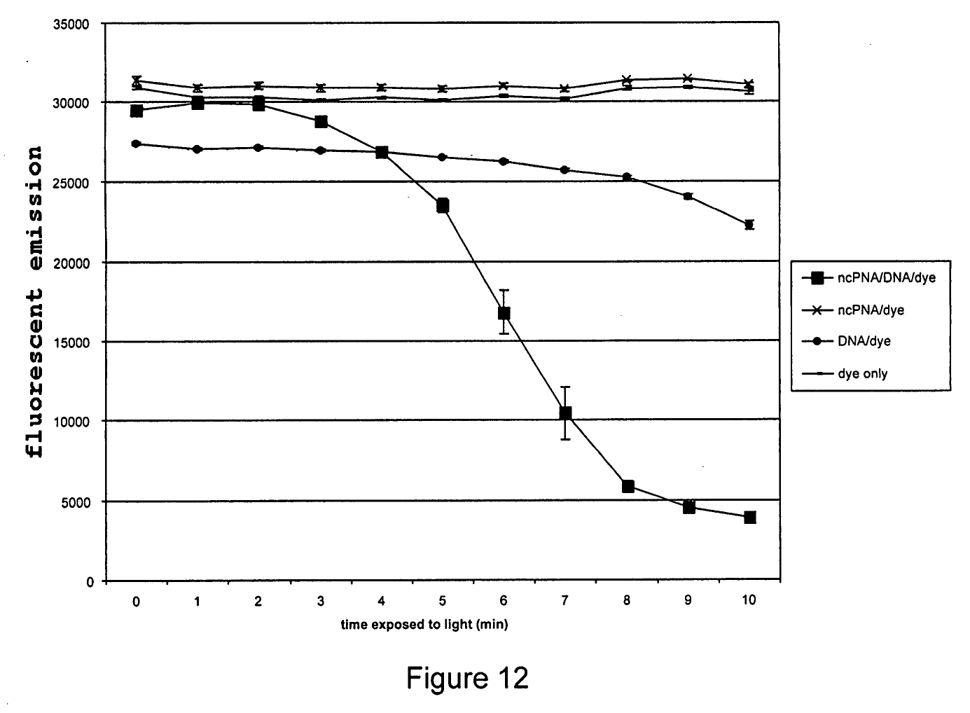

The results are graphically displayed in FIG. 12. The dye only control reaction mixture () and ncPNA/dye control reaction mixture (×) exhibited greatly enhanced stabilization of the zero time fluorescent emission (referred to here as the background signal) by showing virtually no reduction in fluorescence. The DNA/dye control reaction mixture showed a small reduction in fluorescence, however the reduction was negligible through at least seven minutes of light exposure. The test reaction mixture had a fast rate of decrease in fluorescent emission, starting within the third minute,.The addition of the alcohol in tandem with surfactant correlates with the enhanced dye stabilization; as illustrated by negative controls in FIGS. 11, for example.

### Example 11

This example illustrates a fast method for preparing a cellular sample for testing whether a particular target polynucleotide is present. Reaction buffer (0.5 mM phosphate) containing 0.05% NP-40 was used to permeabilize and/or lyse fresh overnight cultures of E. coli and B. cereus grown in tryptic soy broth. Tests from the diluted samples were prepared by using 300 μl from each culture sample. Samples were spun down to a pellet and supernatant removed. Pellets were resuspended in 390 μl of reaction buffer and incubated at room temperature for 10 minutes before use in the test reaction. Each test sample contained 10 pmoles of 16S nucleic acid analog (ncPNA) probe (5′-ACT GCT GCC TCC CGT AG-3′ [SEQ ID NO:8]) or 10 pmoles of HCV-specific biotinylated nucleic acid analog (ncPNA) probe (5′ Bio-(o)₁₀-CGCAGACCACTA 3′ [SEQ ID NO:35]), 5 μl of each lysate dilution test and 4 nmoles of 3,3′-diethylthiacarbocyanine iodide (DTCC dye). The so-constituted test samples were diluted to a 50 μl total reaction volume with 5 mM tw80 buffer. Tests were done either with 16S ncPNA as probe, which should detect the E. coli and B. cereus bacteria, and a non-specific HCV probe that should not recognize any bacterial DNA in this system. The positive control well contained 16S ncPNA probe, 10 ng of isolated E. coli DNA, dye and buffer. The probe only well contained either the HCV ncPNA probe or 16S ncPNA probe, and dye and buffer. The dye only well contained dye and buffer.

ncPNAs were reconstituted in DNase-, RNase-free water to 100 μM stock and further diluted to 2 μM working stocks. The DTCC dye was dissolved in DMSO to 8 mM stocks. This was further diluted to 2 mM working stock in 5 mM phosphate buffer (pH 5.5)+NP-40 0.05%. An initial fluorescence reading was taken at time zero in the Tecan Genios microplate reader with the wavelengths set at 535 nm for excitation and 590 nm for emission. The samples were then exposed to a light stimulus using the Aurora 50/50 for 1 minute intervals with fluorescence reading being taken after each exposure for 10 minutes.

The data were used to calculate the percent change in the test wells containing bacterial lysate and either the HCV ncPNA probe or the 16S ncPNA probe compared to the reaction well containing dye and the analogous HCV ncPNA probe or 16S ncPNA probe. By so comparing the percent change in fluorescence readings after differing numbers of minutes of light exposure, the rate of change of fluorescence emission was determined (which is directly related to the presence of target molecules).

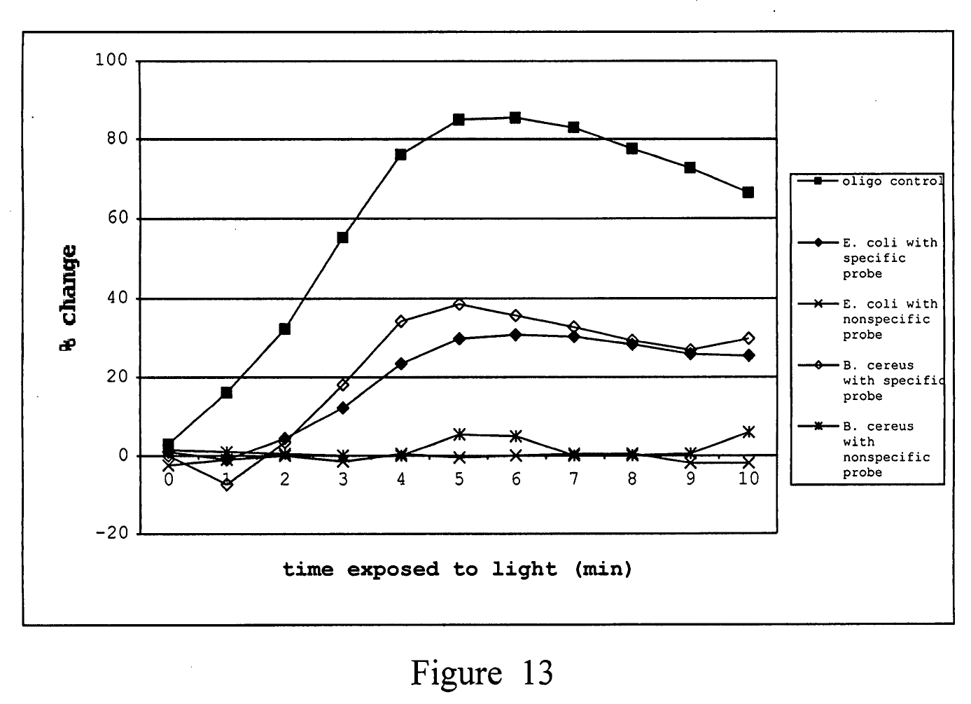

The results are graphically presented in FIG. 13. A strong signal can be seen from the positive control reaction containing bacterial DNA. Test samples containing the 16S ncPNA probe with either E. coli or B. cereus lysates produced robust signals as well, which demonstrates that the method of the present invention is well-suited for bacterial identification using suitable sequences of nucleic acid analogs. Test samples with bacterial lysate and the non-specific HCV ncPNA probe remained at background levels, indicating that one should not expect false-positive results from the herein described method despite the use of crude bacterial lysates for the samples that were tested. Accordingly, the permibilization/lysis buffer producing a crude lysate is sufficient for the detection method disclosed herein.

### Example 12

This example further explores using a crude bacterial lysate as the source of a sample for testing whether a target polynucleotide is present.

Reaction buffer (0.5 mM PO₄) containing 0.05% NP 40 was used to permeabilize and/or lyse diluted aliquots of fresh overnight cultures of E. coli cells that were grown in tryptic soy broth. A sample from the culture was serial diluted and plated on LB agar plates for later colony counts to determine the cell concentration. Analogous serial dilutions of E. coli cells (10 fold dilutions) were made in tryptic soy broth. A “zero” sample contained broth only. Samples were prepared by taking 250 μl of each μldiluted sample, centrifuging to pellet the cells, and removing supernatant. Pellets were resuspended in 390 μl of buffer and incubated at room temperature for 10 minutes before an aliquot thereof was used in the test reaction. Each test sample contained 10 pmoles of 16S ncPNA probe (5′ ACT GCT GCC TCC CGT AG 3′ [SEQ ID NO:8]), 5 μl of each lysate dilution, and 4 nmoles of 3,3′-diethylthiacarbocyanine iodide (DTCC dye); placed in a 50 μl total reaction volume using 5 mM PO₄/NP-40. The positive control well contained 16S ncPNA probe, complementary oligo, dye and buffer. The probe only well contained probe, dye and buffer. The dye only well contained dye and buffer.

ncPNAs were reconstituted in DNase- and RNase-free water to 100 μM stock and further diluted to 2 μM working stocks. The DTCC dye was dissolved in DMSO to 8 mM stocks. This was further diluted to 2 mM working stock in 5 mM phosphate buffer (pH 5.5) +0.05% NP 40. An initial fluorescence reading was taken at time zero in the Tecan Genios microplate reader with the wavelengths set at 535 nm for excitation and 590 nm for emission. The samples were then exposed to a light stimulus using the Aurora 50/50 for 2 minute intervals with fluorescence reading being taken after each exposure for 20 minutes.

The data were used to calculate the percent change in the reaction test well containing the ncPNA probe and the diluted bacterial lysate compared to the reaction well containing only the ncPNA probe/dye.

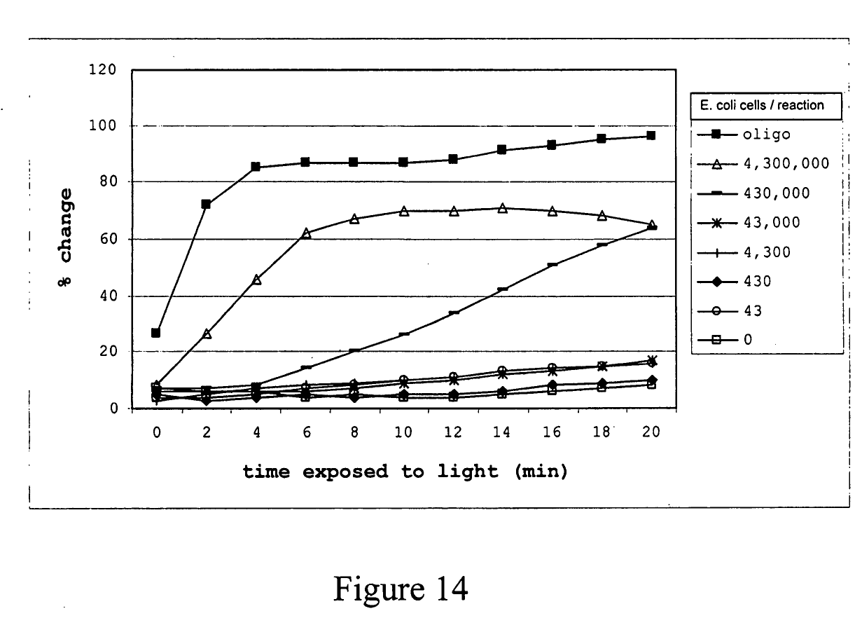

FIG. 14 graphically displays the results. The samples of bacteria that were tested contained the following numbers of bacterial cells: 4.3 million (Δ); 430,000 (); 43,000 (*); 4,300 (|); 430 (♦); 43 (∘); and 0 (□). Additionally a positive control (▪) was included, which had a very strong signal. Similarly, the signal from a reaction mixture having 4,300,000 cells was also very clear, and the signal from a reaction mixture using 430,000 cells, while still clear, departed from the robustness of the signal from a positive control reaction mixture. Counts of 43,000 cells and below resulted in data that was virtually indistinguishable from background levels. These results demonstrate the ability of the method to quantitate in a system of crude cellular lysates.

### Example 13

This example illustrates further the combined effect of Tween 80 and methanol in the reaction buffer, and compares the effect to that of methanol alone.

The effect of methanol in conjunction with Tween 80 at greatly increasing the signal-to-noise ratio in a reaction was described above. (See Example 10). The combined effects with Tween 80 are much greater than when the same concentration of methanol is used alone. Two reactions were made, one with Tween 80 and another without. The modified buffers were made having the following content: (1) 5 mM PO₄ buffer plus 0.05% Tween 80 with 14% methanol; and (2) 5 mM PO₄ buffer with just 14% methanol. Reactions were run using a white 96-well flat-bottom microtiter plate (Greiner) and the Aurora 50/50 fluorescent light at half its intensity.

Reactions were set up in triplicate and loaded in the wells of the 96-well microtiter plate. Reaction mixes were made from the following: 7.5 mM Dye stock, 100 μM ncPNA stock, (5′ Bio-ooooo-GATAGTGGGATTGTGCGT 3′ [SEQ ID NO:1]), 100 μM complementary oligo set (5′ GATAGTGGGATTGTGCGT 3′ [SEQ ID NO:1], 5′ ACGCACAATCCCACTATC 3′ [SEQ ID NO:20]); 1 μl of each exactly complementary oligo pair was mixed equally in annealing buffer (Sambrook, et al) at a concentration of 2 μM and heated to 95° C. for 5 minutes, then cooled to room temperature. The ncPNA were diluted to 2 μM working stock.

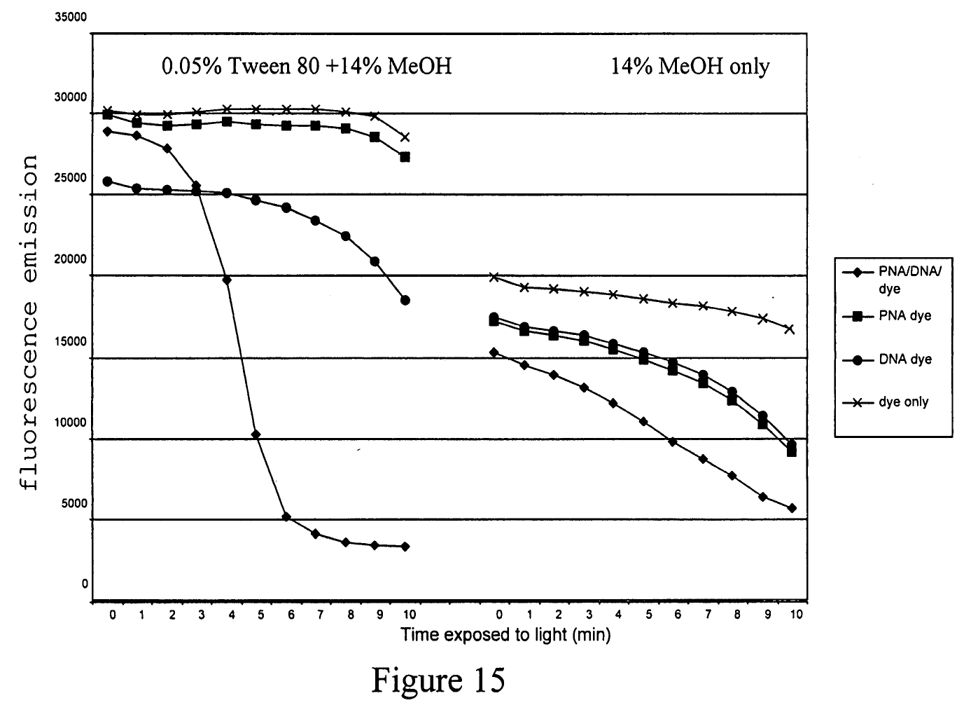

A master dye buffer mix was made by adding 36 μl of 0.75 mM dye to 684 μl of 5 mM PO₄ buffer plus 0.05% Tween® 80 with 14% methanol. The solution was mixed and 160 μl was dispensed to each of 4 wells of a strip tube (Perkin Elmer, Catalog #N801-0580). In the first tube, 20 μl of 2 μM ncPNA and 20 μl of DNA were mixed, making a test reaction mixture. Additionally, these different negative controls were tested in parallel: in a second tube 20 μl of ncPNA and 20 μl of buffer was added making a ncPNA/dye only reaction mixture, in a third tube 20 μL of oligo and 20 μl of buffer was added making the oligo/dye only reaction mixture, and to a forth tube 40 μl of buffer only was added making the dye only reaction mixture. An identical set of reaction mixtures was made as above, but 5 mM PO₄ buffer plus 14% methanol (without Tween® 80] was used. The solution was mixed and 50 μl of each reaction mix was dispensed into 4 wells of the white plate. The plate was placed in the Tecan Genios microplate reader and an initial fluorescence was read without light exposure. Samples were exposed to the Aurora 50/50 fluorescent light at half its light intensity, as above, and readings were taken after every minute of light exposure for 10 minutes. The data were recorded as presented graphically in FIG. 15.

Fluorescent emission at T₀ was greater for reaction mixtures containing the surfactant/alcohol combination in the reaction buffer as compared to reaction mixtures not containing the surfactant/alcohol combination in the reaction buffer. Moreover, all negative control reaction mixtures (PNA+dye, ▪; DNA+dye, ; and dye only, ×) showed increased stability of the fluorescence signal over time, which translates to reduced background noise compared to target polynucleotide, when methanol is used in conjunction with Tween® 80 (left graph set). The dye only and ncPNA/dye reaction mixtures exhibited very little change in fluorescense signal over time. The DNA/dye reaction mixture exhibited minor change in fluorescence signal over time while the test reaction mixture exhibited a very rapid reduction in fluorescence signal over time.

When 14% methanol is used alone there is a much lower relative change in fluorescence signal of the test reaction mixture as compared to the change in fluorescence signal of all control reaction mixtures.

### Example 14

This example illustrates one embodiment of the present invention for determining the presence of a pathogen in a sample.

Samples containing or not containing genomic nucleic acid (NA) isolated from Mycobacterium tuberculosis (MTB CDC 1551) were prepared and tested using a protocol of the present invention. Accordingly, the samples were used in generating reaction mixtures that further included a dye and a nucleic acid analog that specifically hybridizes to MTB DNA. Optical properties of the reaction mixture before and after exposure to a light source were observed.

The concentration of isolated MTB NA (in water for freezer storage) was initially quantified by converting the absorbance of the NA solution at 260 nm (measured with a Hewlett-Packard Model No. HP8452A diode-array spectrophotometer) using standard methods. For the reaction mixtures, the NA was diluted down to a concentration of 0.08 ng/μL in molecular biology grade water (Hyclone, catalog #SH30538.03).

At this point, a 50 μM PNA probe mix was prepared from freezer stocks. Sequences used were Sequence ID NO. 42, 43, 44, 45, 46 and 47. Freezer stocks (at ˜200 μM in H₂O) were put in a hotblock at 65° C. for five minutes, and a 50 μM solution of each ncPNA was prepared by diluting the freezer stock ncPNA in molecular biology grade H₂O (Hyclone, catalog #SH30538.03). Equal volumes of each 50 μM ncPNA solution were added to create a 50 μM total PNA concentration, i.e., 8.33 μM per ncPNA. This 50 μM PNA probe mix was put in the hotblock at 65° C. for five minutes, and was then further diluted in phosphate buffer (10 mM) with surfactant to a final concentration of 320 nM. The 320 nM PNA probe mix was placed in a water bath at 65° C. for five minutes, removed, and given 30 minutes to cool to room temperature.

Dye solution was prepared by diluting 7.5 mM 3,3′-diethylthiacarbocyanine (“DiSC₂(3)”) (solubilized in DMSO) into phosphate buffer with surfactant to a working concentration of 36 μM.

Serial 1:2 dilutions of the 0.08 ng/μL MTB NA (in water) were prepared down to a low concentration of 0.0025 ng/μL; referred to herein as “DNA standards”. Aliquots of 25 μL of each of the DNA standards were dispensed into a 384 well white/clear plate (NUNC, catalog #242763) to set up a concentration curve with six replicates in individual columns on the plate. The columns were set up as follows: Rows 1-6 were assay wells with 2 ng, 1 ng, 0.5 ng, 0.25 ng, 0.125 ng, and 0.0625 ng MTB DNA per well, Row 7 was a control well with 0 ng DNA and Row 8 was a control well with 2 ng MTB DNA.

Because of the sensitivity of the optical measurements, care was taken to ensure that the surfaces of the solutions in the microtiter plate were uniform. The solutions were dispensed using a reverse-pipetting technique described by B. Brando et al. (CYTOMETRY 42:327 (2000)). Briefly, the technique involves pushing the plunger on a mechanical pipettor past the first stop for the initial reagent draw, and pushing the plunger only to the first stop for dispensing, ensuring that a small volume of liquid remains in the pipettor after dispensing. The assay microplate was briefly centrifuged at 500 RPM (34×g) in a Sorvall benchtop centrifuge (Model RT6000D) after the addition of each reagent type.

After the DNA standards were dispensed into the microplate, and the microplate was centrifuged, two dye mixtures were then prepared. The first control dye solution was prepared by mixing equal volumes of 36 μM DiSC₂(3) with phosphate buffer (with surfactant.) This 18 μM DiSC₂(3) control solution was mixed by inversion in a 15 mL conical tube, and poured into a reagent reservoir. A 25 μL aliquot of the control dye solution was then dispensed to each well along Row 8 [H] of the microplate. The second, dye+PNA probe mix was prepared by mixing equal volumes of 36 FM DiSC₂(3) with the 320 nM PNA probe mix. This 18 μM DiSC₂(3) +160 nM PNA probe mix was mixed by inversion in a 15 mL conical tube, and poured into a reagent reservoir. A 25 μL aliquot of the dye+PNA probe mix was then dispensed to each well along Rows 1-7 [A-G] of the microplate. The microplate was then briefly centrifuged at 500 RPM.

The microplate was inserted into the Tecan Safire² monochromator-based microplate reader. The microplate was subjected to a 10 second medium-intensity orbital shake, followed by a 600 second settle time, followed by an absorbance measurement at 556 nm. The absorbance measurement was performed with a bandwidth of 20 nm, with 12 reads per well. After the initial optical measurement, the plate was removed from the spectrofluorimeter, and subjected to photoactivation for a two minute interval. The light for photoactivation was provided by a solid-state activator providing illumination with a peak wavelength of 470 nm and a power density of 2 mW/cm², as measured with a laser-based power meter having the tradename LaserCheck™ (Coherent, Inc., Santa Clara, Calif.; catalog #0217-271-00). The microplate was reinserted into the reader and each well was measured as indicated above, however with a shortened orbital shake and settle time of one second each. This two minute photoactivation followed by absorbance detection cycle was performed over a period of 10 to 44 minutes total exposure to light.

### Example 15

This example sets forth tests conducted to assess the effects of different detergents on diagnostic reactions of the present invention.

In a 96-well white with clear bottom NUNC microtiter plate, 100 μL of a 5 mM phosphate buffer (pH 5.5) was mixed with one of a collection of detergents (also referred to as surfactants), which are listed in Table 8.

TABLE 8

| Trade or Common Name of | | Type of |
|---|---|---|
| Detergent Studied* | Synonyms; Molecular Formula | Detergent |
| CHAPS (100 mg) | 3-[(3- | zwitterionic |
| | cholamidopropyl)dimethylammonio]- | |
| | 1-propanesulfonate hydrate | |
| octyl-β-glucoside (100 mg) | octylglucopyranoside | nonionic |
| octyl-β-D-thioglucoside (100 mg) | octylthioglucopyranoside; OTG | nonionic |
| Surfact-Amps ™ X-100 (a 10% | octylphenol (ethoxylate)ₙ where n is | nonionic |
| aqueous solution of Triton ® X- | 9 or 10 on average | |
| 100) | | |
| Surfact-Amps ™ X-114 (a 10% | octylphenol (ethoxylate)ₙ where n is | nonionic |
| aqueous solution of Triton ® X- | 7 or 8 on average | |
| 114) | | |
| Surfact-Amps ™ NP-40 (a 10% | [octylphenoxy]polyethoxyethanol | nonionic |
| aqueous solution of Nonidet P- | | |
| 40) | | |
| Surfact-Amps ™ 20 (a 10% | polyoxyethylene sorbitan | nonionic |
| aqueous solution of Tween ® 20) | monolaurate; polysorbate 20; | |
| | C₅₈H₁₁₄O₂₆ | |
| Surfact-Amps ™ 80 (a 10% | polyoxyethylenesorbitan monooleate; | nonionic |
| aqueous solution of Tween ® 80) | polysorbate 80 | |
| Surfact-Amps ™ 35 (a 10% | polyoxyethylene monolauryl ether; n | nonionic |
| aqueous solution of Brij ® 35) | ca. 23 | |
| Surfact-Amps ™ 58 (a 10% | polyethylene oxide hexadecyl ether | nonionic |
| aqueous solution of Brij ® 58) | | |

*The full collection of detergents studied was purchased from Pierce Biotechnology, Inc., Rockford, IL; catalog #28340.

Each of the “Surfact-Amps” detergents was further diluted with 5 mM phosphate buffer (pH 5.5) to a final concentration of 0.05%. The 100 mg quantities of CHAPS, octyl-β-D-glucoside, and octyl-β-D-thioglucoside were respectively dissolved in 5 mM phosphate buffer (pH 5.5) to a final concentration of 0.05 mg/mL (w/v). Also included with the phosphate/detergent solution was the dye dipropylthiacarbocyanine iodide (DiSC₃(3)), which was included at a final concentration of 18 μM. A comparison was made between those phosphate/detergent solutions with 100 nM (final concentration) of a pre-annealed PNA:DNA hybrid to those same phosphate/detergent solutions without the PNA:DNA hybrid. The PNA:DNA hybrid used was formed by the combination of PNA sequence biotin-(oo)-GATAGTGGGATTGTGCGT [SEQ ID NO:1] and its complementary DNA oligonucleotide sequence 5′ ACGCACAATCCCACTATC 3′ [SEQ ID NO:20]. Also included in the study were phosphate solutions with no detergent, which were with or without the PNA:DNA hybrid. The Aurora 50/50 light was used for photoactivation, with fluorescence readings (excitation 540 nm, emission 585 nm) taken at T₀ and 2 minute intervals out to 20 minutes using a dual-monochromator, multi-detection microplate reader known as Spectramax M5 (sold by Molecular Devices Corporation, Sunnyvale, Calif.). The Spectramax M5 microplate reader generated data points in relative fluorescence units (RFUs), which were recorded and presented in the tables below.

Table 9 presents the fluorescence data from light-exposed reaction mixtures that included 18 μM DiSC₃(3) with 0.1 μM PNA:DNA in 5 mM phosphate buffer with different detergents (0.05%).

TABLE 9
Data generated from reaction mixtures containing NAA/TP and the
identified detergents or water.

| Time | Relative Fluorescence Units (RFUs) at Stated Minutes of Light Exposure | | | | | | | | | | |
|---|---|---|---|---|---|---|---|---|---|---|---|
| exposed to | | octyl-B- | | | | | | | | | |
| light (min) | CHAPS | glucoside | 1-s-B-thioglucopyranoside | Tween20 | Tween80 | NP40 | TritonX100 | TritonX114 | Brij 35 | Brij 58 | H₂O |
| 0 | 3171 | 2949 | 3106 | 8623 | 9147 | 12095 | 10841 | 7876 | 8564 | 9364 | 3036 |
| 2 | 2153 | 2106 | 2237 | 7283 | 7737 | 10242 | 9407 | 6706 | 7394 | 8154 | 2112 |
| 4 | 1275 | 1384 | 1593 | 5583 | 5923 | 8324 | 7785 | 5147 | 6023 | 6621 | 1585 |
| 6 | 396 | 746 | 829 | 3438 | 3846 | 6221 | 6065 | 3323 | 4382 | 4879 | 1109 |
| 8 | 116 | 246 | 337 | 1376 | 1643 | 4178 | 4357 | 1548 | 2300 | 3077 | 732 |
| 10 | 102 | 106 | 124 | 544 | 690 | 2190 | 2830 | 1097 | 1206 | 1368 | 452 |
| 12 | 93 | 87 | 103 | 385 | 460 | 1447 | 1487 | 928 | 649 | 751 | 308 |
| 14 | 85 | 77 | 91 | 328 | 389 | 1120 | 1011 | 852 | 483 | 548 | 197 |
| 16 | 82 | 66 | 82 | 301 | 352 | 972 | 818 | 789 | 418 | 477 | 135 |
| 18 | 78 | 62 | 76 | 279 | 322 | 885 | 731 | 731 | 381 | 427 | 94 |
| 20 | 75 | 59 | 73 | 263 | 306 | 832 | 670 | 699 | 357 | 403 | 79 |

Each detergent gave a different initial T₀ emission reading, but all behaved similarly in the rates of change in fluorescence upon photoactivation when the PNA:DNA hybrid was present. Based on the data set forth above, from T₀ to T₈, i.e., the first eight minutes of light exposure, the rates of change in fluorescence in the reaction mixture were each between nearly 800 and about 1000 RFU per minute. In contrast, without a detergent included in the reaction mixture, the rate of change over the same eight minutes was about 285 RFU per minute, i.e., about one-third the rate of the detergent-containing reaction mixtures. Because of the approximately three-fold increased rate of change exhibited by the detergent-containing protocol, one can perceive a difference in optical property with the naked eye in a 50 μl to 100 μl reaction mixture within a minute or two, and certainly within five minutes, of the start of photoactivation at ambient temperature.

Table 10 illustrates the percent change between the “ncPNA:DNA” reaction (data in Table 9) and the “dye only” reaction (data in Table 11), relative to the “dye only” reaction at each time point. A remarkable similarity of effect is seen for all tested detergents. The presence of and type of detergent in the reaction affects the time at which maximal percent change between reactions containing dye only and reactions containing dye with ncPNA:DNA occurs. All reactions with detergent (at 0.05%) gave a greater percent change than reactions without detergent relative to dye only reactions.

TABLE 10
Comparative analysis between the results of reaction mixtures after
given time of light exposure and having the identified detergents or water in the
presence of the NAA/TP or not, showing the percentage difference in accordance with
the following formula: [[(RFU_{Table C})) − (RFU_{Table A})] ÷ RFU_{Table C}] × 100.

| | Percent Change Relative to “Dye Only” | | | | | | | | | | |
|---|---|---|---|---|---|---|---|---|---|---|---|
| Time | Relative Fluorescence Units (RFUs) at Stated Minutes of Light Exposure | | | | | | | | | | |
| exposed to | | octyl-B- | 1-s-B- | | | | | | | Brij | |
| light (min) | CHAPS | glucoside | thioglucopyranoside | Tween20 | Tween80 | NP40 | TritonX100 | TritonX114 | Brij 35 | 58 | H₂O |
| 0 | 3.8 | 5.2 | 1.9 | 0.6 | 1.0 | 0.7 | 0.4 | 1.8 | 0.7 | 2.5 | 5.8 |
| 2 | 27.3 | 23.3 | 22.4 | 9.7 | 9.0 | 7.4 | 6.3 | 5.6 | 8.6 | 8.7 | 4.8 |
| 4 | 52.1 | 29.8 | 24.1 | 26.6 | 26.1 | 20.4 | 18.0 | 19.6 | 20.9 | 20.9 | 10.3 |
| 6 | 81.0 | 53.9 | 52.0 | 50.8 | 49.5 | 37.6 | 33.1 | 43.7 | 39.2 | 38.1 | 18.7 |
| 8 | 93.9 | 81.6 | 77.7 | 79.5 | 77.7 | 56.6 | 50.3 | 71.8 | 66.4 | 59.2 | 27.1 |
| 10 | 93.8 | 89.5 | 89.6 | 91.6 | 90.4 | 76.7 | 66.6 | 78.5 | 82.1 | 81.6 | 36.1 |
| 12 | 93.9 | 89.7 | 89.8 | 93.9 | 93.4 | 84.3 | 82.1 | 80.6 | 90.1 | 89.7 | 37.6 |
| 14 | 93.7 | 88.7 | 88.9 | 94.7 | 94.3 | 87.6 | 87.5 | 80.8 | 92.4 | 92.2 | 42.8 |
| 16 | 93.2 | 87.1 | 87.2 | 94.9 | 94.7 | 89.1 | 89.7 | 80.8 | 93.2 | 93.1 | 39.5 |
| 18 | 92.8 | 84.7 | 85.5 | 95.2 | 95.0 | 89.8 | 90.4 | 80.7 | 93.7 | 93.7 | 35.6 |
| 20 | 92.1 | 81.4 | 83.0 | 95.3 | 95.2 | 90.3 | 91.2 | 80.7 | 93.9 | 93.9 | 19.9 |

Identical experiments containing the same detergents but excluding the PNA:DNA hybrid demonstrated the stabilizing effect that certain detergents have on the dye, and thus on the optical property of the reaction mixture. The data was generated using 540 nm exposure of the reaction mixture for excitation of the dye and reading of emitted fluorescence at 585 nm. The data are set forth in Table 11, presented below.

TABLE 11
Data generated from reaction mixtures containing the identified detergents
or water without inclusion of a NAA/TP.

| Time | Relative Fluorescence Units (RFUs) at Stated Minutes of Light Exposure | | | | | | | | | | |
|---|---|---|---|---|---|---|---|---|---|---|---|
| exposed to | | octyl-B- | | | | | | | | | |
| light (min) | CHAPS | glucoside | 1-s-B-thioglucopyranoside | Tween20 | Tween80 | NP40 | TritonX100 | TritonX114 | Brij 35 | Brij 58 | H₂O |
| 0 | 3297 | 3110 | 3164 | 8674 | 9244 | 12010 | 10880 | 7737 | 8622 | 9600 | 3222 |
| 2 | 2959 | 2744 | 2883 | 8061 | 8500 | 11056 | 10041 | 7102 | 8086 | 8931 | 2219 |
| 4 | 2662 | 1971 | 2098 | 7606 | 8012 | 10462 | 9495 | 6404 | 7617 | 8372 | 1767 |
| 6 | 2086 | 1618 | 1727 | 6995 | 7617 | 9966 | 9067 | 5904 | 7203 | 7878 | 1365 |
| 8 | 1895 | 1333 | 1515 | 6714 | 7371 | 9627 | 8774 | 5493 | 6851 | 7535 | 1004 |
| 10 | 1650 | 1008 | 1185 | 6488 | 7163 | 9385 | 8483 | 5096 | 6731 | 7430 | 708 |
| 12 | 1518 | 846 | 1002 | 6285 | 6992 | 9200 | 8322 | 4789 | 6531 | 7283 | 493 |
| 14 | 1350 | 678 | 817 | 6133 | 6853 | 9065 | 8090 | 4432 | 6311 | 7058 | 344 |
| 16 | 1203 | 513 | 646 | 5950 | 6592 | 8890 | 7918 | 4120 | 6190 | 6921 | 223 |
| 18 | 1071 | 409 | 523 | 5776 | 6483 | 8687 | 7650 | 3796 | 6021 | 6750 | 146 |
| 20 | 960 | 317 | 431 | 5641 | 6386 | 8546 | 7582 | 3616 | 5853 | 6626 | 98 |

While a decrease in fluorescence was seen in all of the reactions using the various detergents, or no detergent, the fastest rate of change of the fluorescence was no greater than about a third of the rate of change noted above for reactions where the PNA:DNA hybrid was present. Reactions with the two glucoside derivatized detergents, the only zwitterionic detergent (i.e., CHAPS), or water behaved similarly: These reaction mixtures had T₀ readings that were substantially less than those of the other reactions that included Triton® X, Tween3, and NP-40 non-ionictype detergents. Furthermore, the low level of fluorescence that was present in the glucosides or CHAPS degraded further to near zero over the 20 minute exposure to light. Other dyes were tested, including DiSC₄(3) and DiSC₅(3), and exhibited similar results,

### Example 16

This example illustrates an embodiment of the present invention where an increasing fluorescent signal correlates to a decreasing absorbance of the reaction mixture, thereby providing a simple method for assessing a given reaction.

The top of a 96-well microtiter plate (Nunc #265302) was sealed and the optical bottom was coated with fluorescent yellow paint (Rust-Oleum®, catalog #1942). It is believed that any other optically-identified coating or, in the alternative, an optically-identified inclusion in the material of the reaction vessel or in the reaction mixture itself would work equally well. One simple alternative approach to painting of the reaction vessel, for example, includes, for example, affixing tape or plastic to a surface of the reaction vessel.

ncPNA probe and oligonucleotide complement (“oiDNA”) solutions were prepared by diluting 100 μM stock solutions 1:50 in ddH₂O (Nanopure), resulting in a 2 μM solution of biotinylated ncPNA [SEQ ID NO:1] and a 2 μM solution of the oiDNA that is complementary to the ncPNA [SEQ ID NO:20].

Two master mixes were then prepared: One negative control reaction mixture without target nucleic acid or ncPNA and one test reaction mixture containing the ncPNA and target nucleic acid. The negative control reaction mixture was prepared by adding 24 μL DiSC₂(3) (at 0.75 mM in 10% methanol) and 84 μL of 10% methanol to 492 μL reaction buffer (10 mM PO₄+0.05% Tween® 80). The test reaction mixture was prepared by adding 24 μL DiSC₂(3), 60 μL ncPNA, 60 μL oiDNA and 84 μL 10% methanol to 372 μL of buffer. Each mixture was then dispensed to 12 wells of the painted plate, 50 μL per well.

The fluorescence (excitation at 485 nm, emission at 535 nm, corresponding to the fluorescence of the yellow paint on the plate) of each well at zero time (T₀; no light exposure prior thereto) was then read using a Tecan GENios microplate reader. Average fluorescent signal was determined for each control or experimental reaction, standard deviations were calculated, and the data were recorded.

Reactions were then activated with the Aurora 50/50 illuminator for 1 minute. Fluorescence was measured with the same parameters as the T₀ measurement. This cycle of photoactivation followed by an immediate fluorescence measurement was continued out to 10 minutes of exposure to light. The data are provided in the following table:

TABLE 12
Fluorescence data from test reaction mixtures and negative control reaction
mixtures, where the fluorescence measured relates to fluorescent material painted on
the outside of the reaction vessels.

| | | | | RFU* after stated time of light exposure (min) | | | | | | | | | | |
|---|---|---|---|---|---|---|---|---|---|---|---|---|---|---|
| | DNA | PNA | DYE | 0 | 1 | 2 | 3 | 4 | 5 | 6 | 7 | 8 | 9 | 10 |
| Test | + | + | + | 7821 | 11526 | 14876 | 19213 | 25229 | 33175 | 35938 | 36793 | 37252 | 37617 | 37545 |
| Reaction | | | | | | | | | | | | | | |
| Negative | − | − | + | 7748 | 8397 | 8928 | 9513 | 10070 | 10615 | 11220 | 11743 | 12271 | 12844 | 13306 |
| Control | | | | | | | | | | | | | | |

*The acronym RFU refers to units of relative fluorescence.

The increasing fluorescence seen in the experimental reaction correlates to a decreasing opacity of the reaction mixture itself, which is perceivable with the naked eye. The decreasing opacity of the test reaction mixture was evident in the reaction vessels as a progressive clearing of color from the reaction mixture, which was noticed as of about one minute of light exposure. In contrast, in the negative control, the substantially constant relative fluorescence units seen in the experiment correlate to the substantially constant opacity of the reaction mixture. Indeed, the negative control reaction mixture appeared to substantially maintain the same intensity of color through the entire time course of the experiment.

The fluorescence noted in the experimental reaction emanates from the painted bottom of the microtiter wells, which wells were identical to those in which the negative controls were run. Accordingly, the characteristic that is actively changing in the reaction is the absorbance, which, as it decreases in the test reaction mixture over time, reveals more of the fluorescent paint coated onto the well. A further conclusion arising from this example is that one does not have to measure an optical property that derives from the chemical state of a dye; instead, one can more simply measure, or, truly, merely notice an uncovering of a second optical property of the reaction mixture and/or reaction vessel that contains the reaction mixture. The optical property of the dye included in the reaction mixture obscures a well-chosen second optical property of the reaction mixture and/or reaction vessel. To the extent that the chemical state of the dye alters in the reaction mixture such that a decreasing concentration of the original dye remains over time of the assay, the optical property contributed by the dye to the reaction mixture diminishes, thereby revealing the presence of the second optical property, which can be used to trigger realization of the presence or quantity of a target polynucleotide.

### Example 17

This example sets forth an investigation of molecular weight changes in a dye over the course of a diagnostic reaction according to the present invention.

A mass spectroscopic analysis of a dye exposed to light in the presence or absence of a ncPNA:DNA hybrid was accomplished, as follows:

Using a 96-well clear, streptavidin-coated microtiter plate (Nalge Nunc International, Rochester, N.Y.; NUNC Immobilizer™ Streptavidin plates), a biotinylated ncPNA (i.e., biotin-(oo)-TGCCTCCCGTAG [SEQ ID NO:9]) was used to capture isolated E. coli DNA. The PNA used in this experiment is specific for a ubiquitous bacterial 16S sequence. To prepare the microplate used in the experiment, the following steps were undertaken: (1) each well was washed three times with 300 μL phosphate-buffered saline solution with 0.05% Tween® 20 (“PBST”); (2) 50 μL aliquots of a solution containing 2.5 μL of 2 μM biotinylated ncPNA (5 pmoles), 5 μL of 10 ng/μL E. coli genomic DNA (50 ng), and 42.5 μL of 5 mM phosphate buffer (pH 5.5) were introduced into wells of the microplate; (3) the microplate was sealed and placed on a rotamixer for 60 minutes (at room temperature) to allow biotin-streptavidin interactions to occur; and (4) the wells were aspirated and washed five times with 200 μL PBST to remove unbound DNA and ncPNA.

A DiSC₂(3) solution was made by diluting an 8 mM (in DMSO) stock solution to 2 mM with 5 mM phosphate buffer (pH 5.5). The dye solution was further diluted to 80 μM with a 5 mM phosphate/0.05% Tween® 20 solution; aliquots of the diluted dye solution were introduced into each well. The plate was then placed on top of an Aurora 50/50 light for 30 minutes before the solutions were pooled into a 15 mL conical container with an aluminum foil shroud, thereby keeping ambient light from the contained solution. In order to have enough reaction product for liquid chromatography-mass spectrometry (“LC/MS”) experiments, 12 identical wells were prepared for each reaction and pooled. Also included were control wells with ncPNA only and with DNA only. Only those wells that were exposed to liquid and a ncPNA:DNA hybrid displayed the expected reduction in fluorescence associated with the diagnostic method of the present invention.

The pooled products were then further analyzed by LC/MS using standard methods and instrumentation via the services of a commercial analytical chemistry laboratory. No ncPNA:DNA-specific dye product could be found, which was determined by comparison to LC analysis of products of the “Dye Only” control wells. However, an LC sample fraction collected at 9.8 minutes included a new product of apparent molecular weight 427.1 mass units (“mu”). The original parent dye compound, DiSC₂(3), can be represented by C₂₁H₂₁N₂S₂, which is 365.5 mass units not including the iodide counterion. The new product of 427.1 mu corresponds to an oxygen addition to the sulfur group (+15.56 mu) of the parent dye compound, which subsequently forms an adduct with formic acid (+46.01 mu) during the liquid chromatography run. The new product, DiSC₂(3)ₒₓ, has the chemical name 1-oxo-3,3′-diethylthiacarbocyanine iodide and the chemical formula C₂₁H₂₁N₂OS₂. DiSC₂(3)ₒₓ has two resonance structures as shown in the structures presented in FIG. 16.

A conclusion of this study is that a sulfur atom of DiSC₂(3) is oxidized in the reaction of the present invention where the optical property of the reaction mixture diminishes. Whereas the optical property of interest exhibited at T₀ by a reaction mixture constituted in accordance with the present invention correlates to the dye, the oxidation of the sulfur atom of the dye that is disclosed here correlates to the absence or reduction in the optical property of the reaction mixture post-photoactivation in the presence of the NAA/TP.

### Example 18

This example is illustrative of a smartDNA reaction in a gel matrix, bound to a protein (or large macromolecule). Agarose super-shift assays were performed. A biotinylated ncPNA (biotin-OO-TGCCTCCCGTAG [SEQ ID #9]) was hybridized to a complementary DNA oligonucleotide (5′-CTACGGGAGGCA-3′ [SEQ ID #57]) at a final concentration of 25 μM. Goat anti-biotin antibody (Immunology Consultants Laboratory, ICL) at 1 mg/mL was used undiluted. In a microfuge tube, 1 μL of ncPNA:DNA duplex was mixed with 1 uL antibody and allowed to sit at room temperature for thirty minutes to permit biotin-streptavidin interactions. For comparison, a second and third tube contained 1 μL ncPNA:DNA duplex only or antibody only, respectively. Each of these solutions was mixed with 5 μL of a 50% glycerol solution and loaded into the wells of a 1% agarose (1× TBE) gel containing 2.5 uM DiSC₂(3) (added while the agarose was molten). Electrophoresis proceeded at 50V for 60 minutes in 1× TBE running buffer. An initial time zero photograph was taken of the gel illuminated with a UV transilluminator. The gel was then exposed to a light stimulus Aurora 50/50 for 5 minutes before a second photograph was taken.

Lanes 1 and 2 show “holes” (loss of fluorescence) for a fast migrating species corresponding to a ncPNA:DNA duplex (unbound to antibody) breaking down the dye after 5 minutes of exposure to light. Lane 1 shows a (super-shift) slower migrating “hole” suggesting that the ncPNA:DNA duplex is bound to the antibody through a biotin-streptavidin interaction and that this interaction does not interfere with the photobleaching. Lane 3 (antibody only) shows no photobleaching thereby confirming that the super-shift “hole” in Lane 1 is not due to the antibody.

Future experiments include chemically coupling the ncPNA:DNA duplex to an antibody which is specific for a given antigen.

### Example 19

This example illustrates an embodiment of the present invention with varying lengths of ncPNA.

Six different 17-mer and 12-mer ncPNA probes targeting similar nucleic acid sequences within isolated Mycobacteria tuberculosis (MTB) genomic DNA were tested.

Working solutions of 2 μM ‘12-mer nccocktail' were generated from equal volumes of 2 μM solutions of each individual 12-mer ncPNA. Working solutions of 2 μM ‘17-mer nccocktail’ were generated from equal volumes of 2 μM solutions of each individual 17-mer ncPNA. The PNAs used are set forth in Table 13.

TABLE 13

| | | SEQ ID |
|---|---|---|
| Code Name | PNA Sequence | NO: |
| TB01 | biotin-(OO)-GTCGTCAGACCCAAAAC | 36 |
| TB02 | biotin-(OO)-CGAGAGGGGACGGAAAC | 37 |
| TB03 | biotin-(OO)-TGAACCGCCCCGGCATG | 38 |
| TB04 | biotin-(OO)-ACCAAGTAGACGGGCGA | 39 |
| TB05 | biotin-(OO)-CATCCAACCGTCGGTCG | 40 |
| TB06 | biotin(OO)-ACAAGACATGCATCCCG | 41 |
| TB07 | lysine-CAGACCCAAAAC | 42 |
| TB08 | lysine-CGAGAGGGGACG | 43 |
| TB09 | lysine-TGAACCGCCCCG | 44 |
| TB10 | lysine-ACCAAGTAGACG | 45 |
| TB11 | lysine-CATCCAACCGTC | 46 |
| TB12 | lysine-ACAAGACATGCA | 47 |

Introduced into a 384-well white with white bottom microtiter plate (purchased from Costar) were: 2 μL of a 1 ng/μL solution of MTB genomic DNA (obtained from MRL, Department of Microbiology, Ft. Collins, Colo.), 2 μL of the 2 μM ‘cocktail’ of 12-mer ncPNAs or 2 μL of the 2 μM ‘cocktail’ of 17-mer PNAs, and 16 μL H₂O (Molecular Biology Grade). This solution was vigorously mixed for 5 seconds and allowed to incubate for 10 minutes at room temperature. Thirty microliters of a solution containing 5 mM phosphate (pH 5.5), 0.083% Tween® 80, and 15 μM diethylthiacarbocyanine iodide (DiSC₂(3)) was then added to each well (final concentration of 3 mM phosphate, 0.05% Tween® 80, 9 ρM DiSC₂(3)) and mixed.

An initial To reading (excitation 535 nm, emission 590 nm) was obtained using a Tecan Genios fluorescence microtiter plate reader. Next, the reactive mixtures in the microtiter wells were exposed to light using an Aurora 50/50 light. Fluorescence readings were taken every two minutes out to 30 minutes total light exposure time. To assess non-specific binding of ncPNAs, an identical experiment containing human genomic DNA (isolated from a B cell line (GM14686; Coriell Cell Repositories, Camden, N.J.)) instead of MTB DNA was run in parallel. Control wells containing ncPNA only (no DNA) and DNA only (no ncPNA) were also included. The average fluorescence of four identical reactions was plotted along with standard error bars as a function of light exposure time.

The data indicate that ncPNAs having 12 or 17 nucleotides are usefully employed with the present invention. Whereas the rate of change in fluorescence was indeed faster with the larger ncPNA targeting MTB DNA (see FIG. 18A), a greater level of non-specific activity was shown in data generated using the larger PNA (see FIG. 18B).

The data are consistent with the view that a ‘cocktail’ of 12-mer ncPNAs can drive a diagnostic reaction after incubation with isolated MTB genomic DNA at room temperature. Although a ‘cocktail’ of 12-mer ncPNA correlates to a slower rate than a ‘cocktail’ of 17-mer PNAs, fewer non-specific reactions were detected when the 12-mer PNAs were combined with the unrelated DNA as compared to when the 17-mer PNAs were combined with the unrelated DNA.

### Example 20

This example illustrates the use individual ncPNAs (instead of a “cocktail” of ncPNAs) to detect MTB DNA.

In particular, ncPNA TB10 [SEQ ID NO. 45] and ncPNA TB12 [SEQ ID NO. 47] were used in the protocol set forth in Example 14 to detect isolated genomic MTB DNA at 2, 1, 0.5, 0.25, 0.0625, and 0 ng/50 uL reactions (with the exception that the “DNA Standards” were diluted in 10 mM Tris-Cl (pH 7.2), 1 mM EDTA, 0.05% Tween-80 buffer).

Briefly, 25 uL of the “DNA Standards” (0.08 ng/uL, 0.04 ng/uL, 0.02 ng/uL, 0.01 ng/uL, 0.005 ng/uL, 0.0025 ng/uL, and 0 ng/uL) were dispensed into a 384-well white with clear bottom microtiter plate (NUNC, catalog #242763). From an 8.3 uM stock (prepared in H₂O), ncPNA TB10 (or ncPNA TB 12) was diluted to a working concentration of 53.3 nM in Tris-Cl (pH 7.2), 1 mM EDTA, 0.05% Tween® 80 buffer. This solution was mixed with equal volumes of a solution containing 36 uM DiSC₂(3) in Tris-Cl (pH 7.2), 1 mM EDTA, 0.05% Tweeno 80 buffer. Twenty-five microliters of this mixture were added to the “DNA standard” in the microtiter, followed by centrifugation of the plate, shaking, and incubation at room temperature as per the protocol in Example 14.

Absorbance measurements at 556 nm were recorded at T₀ and every 2 minutes thereafter, up to 44 minutes of photoactivaton. Photoactivation was done using a solid-state activator providing illumination at 470 nm with a power density of 2 mW/cm². Data was compiled and assessed for the time at which absorbance had reached 50% of initial starting absorbance (defined as the T₅₀% ) as depicted in the table below.

TABLE
Time to reach 50% absorbance, (T_{50%}) in minutes

| amount of DNA per well | | ncPNA TB10 | ncPNA TB12 |
|---|---|---|---|
| 2 | ng | 11.3 | 10 |
| 1 | ng | 14.6 | 12.5 |
| 0.5 | ng | 16.7 | 16.5 |
| 0.25 | ng | 23.2 | 23.7 |
| 0.125 | ng | 28.3 | 21.7 |
| 0.0625 | ng | 32.1 | 28.2 |
| ncPNA Only (No DNA) | | 34.2 | 33.1 |
| 2 | ng DNA Only (No | 38.6 | 33.9 |
| | ncPNA) | | |

The reactions containing ncPNA TB12 had lower T_{50%} times as compared to ncPNA TB10, suggesting faster detection of MTB DNA. This difference in performance between the two ncPNAs could be attributed to either sequence-specificity difference (of the particular ncPNA:DNA duplexes formed), or a targeting ability (strand-invasion into a genomic region), or a target copy number difference of each ncPNA. While ncPNA TB10 has the potential to bind upwards of 20 sequences within the MTB genome (IS6110 transposon sequence of variable copy number), ncPNA TB12 only has a single genomic target (rDNA), but is also specific for MTB rRNA that may be present (residual) in the genomic DNA preparations.

All publications, patents, and patent applications cited herein are hereby incorporated by reference in their entireties, respectively, for all purposes and to the same extent as if each individual publication, patent, or patent application were specifically and individually indicated to be so incorporated by reference. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of the disclosure.

We claim:

1. A method of detecting a target polynucleotide in a sample, comprising the steps of:
(a) producing a reaction mixture comprising the sample, a first nucleic acid or a first nucleic acid analog, and a dye,
wherein the first nucleic acid or the first nucleic acid analog is at least partially complementary to a segment of the target polynucleotide;
(b) exposing the reaction mixture to a light; and
(c) observing the optical property of the reaction mixture at least once after exposure to the light;
wherein the reaction mixture has an optical property that changes in response to the light exposure if the first nucleic acid or first nucleic acid analog and the target polynucleotide are present therein, and
wherein the dye is the compound of formula (I), or a salt or ester thereof: 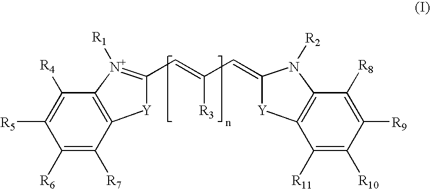
wherein, independently at each occurrence:
R₁ and R₂ are each independently selected from hydrogen, alkyl, alkenyl, alkynyl, heteroalkyl, heteroalkenyl, heteroalkynyl, aryl, acyl, heteroacyl, heteroaryl, hydroxyl, alkoxy, carbonyl, sulfinyl, sulfonyl, and amino groups;
R₃ is selected from the group consisting of hydrogen, alkyl, alkenyl, alkynyl, aryl, heteroalkyl, heteroalkenyl, heteroalkynyl, aryl, acyl, heteroacyl, heteroaryl, aryl, alkyl, heteroarylalkyl, hydroxyl, alkoxy, halo, carbonyl, sulfinyl, sulfonyl, and amino groups;
R₄, R₅, R₆, R₇, R₈, R₉, R₁₀, R₁₁, R₁₂, and R₁₃ are each independently selected from the group consisting of hydrogen, halogen, alkyl, alkenyl, alkynyl, heteroalkyl, heteroalkenyl, heteroalkynyl, aryl, acyl, heteroacyl, heteroaryl, aryl, alkyl, heteroarylalkyl, hydroxyl, alkoxy, carbonyl, sulfinyl, sulfonyl groups;
n is 0 1, 2, 3, 4, or 5; and
Y is —CR₁₂═CR₁₃—, sulfur, or oxygen;

2. The method of claim 1, further comprising the step of:
(d) correlating the detecting of the target polynucleotide with the resultant change in the optical property of the reaction mixture.

3. The method of claim 2, wherein the change in the optical property correlates with a chemical change in the dye.

4. The method of claim 1, wherein the optical property of the reaction mixture is observed at least twice.

5. The method of claim 1, wherein the optical property comprises a first optical property that diminishes after exposure to the light and a second optical property that increases after exposure to the light.

6. The method of claim 5, wherein the reaction mixture is contained in a vessel and further comprises a substance,
wherein the vessel or the substance includes the second optical property,
wherein the substance is not the dye.

7. The method of claim 1, wherein the reaction mixture includes a detergent.

8. The method of claim 7, wherein the sample comprises intact cells prior to being in contact with the detergent.

9. The method of claim 1, wherein the reaction mixture includes an achiral peptide nucleic acid.

10. The method of claim 1, wherein the reaction mixture includes a chiral peptide nucleic acid.

11. The method of claim 1, wherein the length of the target polynucleotide is greater than about 50 bases.

12. The method of claim 1, wherein the reaction mixture further comprises a second nucleic acid, and wherein at least a portion of the second nucleic acid is complementary to a portion of the first nucleic acid that is not complementary to the target polynucleotide.

13. The method of claim 12, wherein the reaction mixture further comprises a third nucleic acid, and wherein one portion of the third nucleic acid is complementary to a portion of the target polynucleotide that is not complementary to the first nucleic acid and wherein another portion of the third nucleic acid is complementary to a portion of the second nucleic acid that is not complementary to the first nucleic acid.

14. The method of claim 13, wherein the first part and the second part of the nucleic acid analog do not overlap.

15. The method of claim 13, wherein the first part and the second part of the target nucleic acid sequence of the target polynucleotide do not overlap

16. The method of claim 1, wherein the first nucleic acid analog is greater than about 4 bases in length and less than about 24 bases in length.

17. The method of claim 1, wherein the second nucleic acid analog is greater than about 4 bases in length and less than about 24 bases in length.

18. The method of claim 16, wherein the first nucleic acid analog is about 12 nucleic acid bases in length.

19. The method of claim 16, wherein the second nucleic acid analog is about 12 nucleic acid bases in length.

20. The method of claim 1, further comprising immobilizing the target polynucleotide and the first nucleic acid analog on a solid substrate.

21. The method of claim 20, wherein the first nucleic acid analog or the second nucleic acid analog is attached to a solid substrate.

22. The method of claim 12, further comprising immobilizing the target polynucleotide and the second nucleic acid analog on a solid substrate.

23. The method of claim 22, wherein the second nucleic acid analog or the second nucleic acid analog is attached to a solid substrate.

24. The method of claim 1, wherein the dye is not selected from the following list: 3,3′-Diethylthiacyanine; 3-Ethyl-9-methyl-3′-(3-sulfatobutyl)thiacarbocyanine; 3,3′-Dimethyloxacarbocyanine; 3-Carboxymethyl-3′,9-diethyl-5,5′-dimethylthiacarbocyanine; 3,3′-Diethylthiadicarbocyanine; 3,3′-Diethylthiatricarbocyanine; 3,3′-Diethyloxacarbocyanine; 3,3′-Diethyloxadicarbocyanine; 3,3′-Dipropylthiadicarbocyanine; 3,3′-Dipropyloxacarbocyanine; 3,3′-Dihexyloxacarbocyanine; 3,3′-Diethyl-2,2′-oxathiacarbocyanine; 1,1′-Diethyl-2,2′-cyanine; 1,1′-Diethyl-2,4′-cyanine; 1,1′-Diethyl-4,4′-carbocyanine; 1,1′-Diethyl-3,3,3′,3′-tetramethylindocarbocyanine; 1,1′-Dipropyl-3,3,3′,3′-tetramethylindocarbocyanine; [5-[2-(3-Ethyl-3H-benzothiazol-2-ylidene)-ethylidene]-4-oxo-2-thioxo-thiazolidin-3-yl]-acetic acid; 1-Butyl-2-[3-(1-butyl-1H-benzo[cd]indol-2-ylidene)-propenyl]-benzo[cd]indolium; 5,6-Dichloro-2-[3-(5,6-dichloro-1,3-diethyl-1,3-dihydro-benzimidazol-2-ylidene)-propenyl]-1,3-diethyl-3H-benzimidazolium; 1,3,3-Trimethyl-2-(2-[2-phenylsulfanyl-3-[2-(1,3,3-trimethyl-1,3-dihydro-indol-2-ylidene)-ethylidene]-cyclohex-1-enyl]-vinyl)-3H-indolium; 4,5,4′,5′-Dibenzo-3,3′-diethyl-9-methyl-thiacarbocyanine; and Thiazole orange.

25. The method of claim 12, wherein R₁, R₂, and R₃ are hydrogen or hydrophobic alkyls, R₄ through R₁₃ are hydrogen, and Y is sulfur.

26. The method of claim 1, wherein the target polynucleotide is at least 50 nucleotides in length.

27. The method of claim 12, wherein the target polynucleotide is at least 50 nucleotides in length.

28. The method of claim 1, wherein the dye is of formula (I), wherein n is 1.

29. The method of claim 28, wherein the dye is of formula (I), wherein n is 1; and wherein Y is sulfur or —CR₁₂═CR₁₃—.

30. The method of claim 28, wherein the dye is of formula (I), wherein n is 1; wherein Y is sulfur or —CR₁₂═CR₁₃—; and wherein R₁ and R₂ are each independently selected from the group consisting of alkyl and alkenyl.

31. The method of claim 12, wherein the dye is of formula (I), wherein n is 1.

32. The method of claim 31, wherein the dye is of formula (I), wherein n is 1; and wherein Y is sulfur or —CR₁₂═CR₁₃—.

33. The method of claim 31, wherein the dye is of formula (I), wherein n is 1; wherein Y is sulfur or —CR₁₂═CR₁₃—; and wherein R₁ and R₂ are each independently selected from the group consisting of alkyl and alkenyl.

34. A method of detecting a target polynucleotide in a sample, comprising the steps of:
(a) producing a reaction mixture comprising the sample, a nucleic acid analog that is complementary to a target nucleic acid sequence of the target polynucleotide, and a dye;
(b) exposing the reaction mixture to a light; and
(c) observing the absorbance of the reaction mixture at least once;
wherein the reaction mixture has an absorbance that changes if the target polynucleotide and the nucleic acid analog form a hybrid therein;
wherein the dye is the compound of formula (I), or a salt thereof: 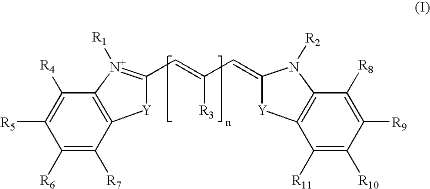
wherein, independently at each occurrence:
R₁ and R₂ are each independently selected from hydrogen, alkyl, alkenyl, alkynyl, heteroalkyl, heteroalkenyl, heteroalkynyl, aryl, acyl, heteroacyl, heteroaryl, hydroxyl, alkoxy, carbonyl, sulfinyl, sulfonyl, and amino groups;
R₃ is selected from the group consisting of hydrogen, alkyl, alkenyl, alkynyl, aryl, heteroalkyl, heteroalkenyl, heteroalkynyl, aryl, acyl, heteroacyl, heteroaryl, aryl, alkyl, heteroarylalkyl, hydroxyl, alkoxy, halo, carbonyl, sulfinyl, sulfonyl, and amino groups;
R₄, R₅, R₆, R₇, R₈, R₉, R₁₀, R₁₁, R₁₂, and R₁₃ are each independently selected from the group consisting of hydrogen, halogen, alkyl, alkenyl, alkynyl, heteroalkyl, heteroalkenyl, heteroalkynyl, aryl, acyl, heteroacyl, heteroaryl, aryl, alkyl, heteroarylalkyl, hydroxyl, alkoxy, carbonyl, sulfinyl, sulfonyl groups;
n is 0 1, 2, 3, 4, or 5; and
Y is —CR₁₂═CR₁₃—, sulfur, or oxygen.

35. The method of claim 34, further comprising the step of:
(d) correlating the detecting of the target polynucleotide with the resultant change in the optical property of the reaction mixture.

36. The method of claim 35, wherein the change in the optical property correlates with a chemical change in the dye.

37. The method of claim 33, wherein the absorbance of the reaction mixture is observed at least twice.

38. The method of claim 33, wherein the optical property comprises a first optical property that diminishes after exposure to light and a second optical property that increases after exposure to light.

39. The method of claim 38, wherein the reaction mixture is contained in a vessel and further comprises a substance,
wherein the vessel or the substance includes the second optical property,
wherein the substance is not the dye.

40. The method of claim 34, wherein the nucleic acid analog is an achiral peptide nucleic acid.

41. The method of claim 34, wherein the nucleic acid analog is a chiral peptide nucleic acid.

42. The method of claim 34, wherein the reaction mixture includes a detergent.

43. The method of claim 42, wherein the sample includes intact cells prior to being in contact with the detergent.

44. The method of claim 34, wherein the nucleic acid analog is greater than about 4 nucleic acid bases in length and less than about 24 nucleic acid bases in length.

45. The method of claim 34, wherein the nucleic acid analog or the target polynucleotide is immobilized on a solid substrate.

46. The method of claim 45, wherein the nucleic acid analog is attached to a solid substrate.

47. The method of claim 34, wherein the nucleic acid analog is about 12 bases in length.

48. The method of claim 34, wherein the target polynucleotide is greater than about 50 bases in length.

49. The method of claim 34, wherein R₁, R₂, and R₃ are hydrogen or hydrophobic alkyls, R₄ through R₁₃ are hydrogen, and Y is sulfur.

50. A composition comprising a surfactant and a dye according to formula (I), or a salt thereof: 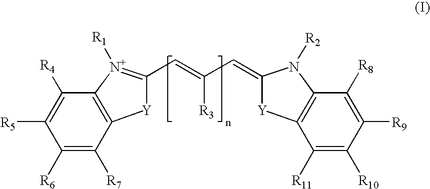
wherein, independently at each occurrence:
R₁ and R₂ are each independently selected from hydrogen, alkyl, alkenyl, alkynyl, heteroalkyl, heteroalkenyl, heteroalkynyl, aryl, acyl, heteroacyl, heteroaryl, hydroxyl, alkoxy, carbonyl, sulfinyl, sulfonyl, and amino groups;
R₃ is selected from the group consisting of hydrogen, alkyl, alkenyl, alkynyl, aryl, heteroalkyl, heteroalkenyl, heteroalkynyl, aryl, acyl, heteroacyl, heteroaryl, aryl, alkyl, heteroarylalkyl, hydroxyl, alkoxy, halo, carbonyl, sulfinyl, sulfonyl, and amino groups;
R₄, R₅, R₆, R₇, R₈, R₉, R₁₀, R₁₁, R₁₂, and R₁₃ are each independently selected from the group consisting of hydrogen, halogen, alkyl, alkenyl, alkynyl, heteroalkyl, heteroalkenyl, heteroalkynyl, aryl, acyl, heteroacyl, heteroaryl, aryl, alkyl, heteroarylalkyl, hydroxyl, alkoxy, carbonyl, sulfinyl, sulfonyl groups;
n is 0 1, 2, 3, 4, or 5; and
Y is —CR₁₂═CR₁₃—, sulfur, or oxygen.

51. The composition of claim 50, wherein the composition comprises a dye according to the structure of formula (II), or a salt or ester thereof, 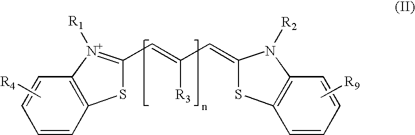
wherein, independently at each occurrence:
R₁ and R₂ are each independently selected from C₁-C₆ alkyl, C₂-C₆ alkenyl, and C₂-C₆ alkynyl;
R₃ is selected from the group consisting of H, C₁-C₆ alkyl, C₂-C₆ alkenyl, and C₂-C₆ alkynyl, C₆-C₁₀ aryl, hydroxyl, alkoxy, halo, carbonyl, sulfinyl, sulfonyl, and amino groups;
n is 1 or 2; and
R₄ and R₉ are each independently selected from the group consisting of H, C₁-C₆ alkyl, C₂-C₆ alkenyl, and C₂-C₆ alkynyl, C₁-C₆ aryl, hydroxyl, alkoxy, halo, carbonyl, sulfinyl, sulfonyl, and amino groups.

52. The composition of claim 50, wherein the dye has the structure according to formula (III): 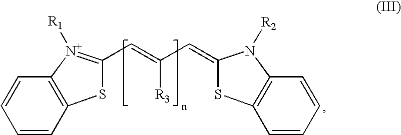
wherein, independently at each occurrence:
R₁ and R₂ are each independently selected from C₁-C₆ alkyl and C₂-C₆ alkenyl;
R₃ is selected from the group consisting of H and methyl; and
n is 1 or 2.

53. The composition of claim 50, further comprising a nucleic acid analog.

54. The composition of claim 50, further comprising a target polynucleotide.

55. A kit for detecting a target polynucleotide, said kit comprising:
(a) one or more nucleic acid analogs at least partially complementary to a target nucleic acid sequence of said target polynucleotide,
(b) one or more dyes;
(c) one or more surfactants; and
(d) instructions that relate to the method of claim 1.

56. A reporter complex comprising a first polynucleotide, a second polynucleotide, and a dye, wherein the first polynucleotide and the second polynucleotide form a hybrid and the reporter complex has an optical property that changes in response to exposure to a light stimulus.

57. The reporter complex of claim 35, wherein the optical property is absorbance.

58. The reporter complex of claim 56, wherein the hybrid is attached to a target binding component.

59. The reporter complex of claim 58, wherein the target binding component is selected from the group consisting of an antibody or fragment thereof, a lectin, or a receptor.

60. The reporter complex of claim 56, wherein one of the polypeptide and the nucleic acid analog is immobilized on a solid substate.

61. The reporter complex of claim 56, wherein the second polynucleotide is a nucleic acid analog.

62. A method for detecting a target molecule in a sample, in which the target molecule and a target binding component bind one another with substantial specificicity comprising:
(a) combining the sample, the target binding component, a first polynucleotide, and a second polynucleotide in a reaction mixture in a vessel,
wherein the first polynucleotide and second polynucleotide form a hybrid and are in contact with the target binding component,
wherein at least one of the target binding component, first polynucleotide, and second polynucleotide, or the target molecule are attached to a solid surface;
(b) washing the reaction mixture;
(c) combining the reaction mixture components that are immobilized on the solid substrate with dye, whereupon the reaction mixture has an optical property that changes if the sample includes the target molecule and it and the target binding component bind one another;
(d) exposing the reaction mixture to light; and
(e) observing the optical property of the reaction mixture at least once;
wherein the dye is a compound of formula (I), or a salt thereof: 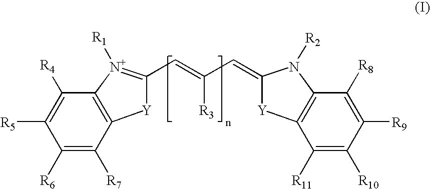
wherein, independently at each occurrence:
R₁ and R₂ are each independently selected from hydrogen, alkyl, alkenyl, alkynyl, heteroalkyl, heteroalkenyl, heteroalkynyl, aryl, acyl, heteroacyl, heteroaryl, hydroxyl, alkoxy, carbonyl, sulfinyl, sulfonyl, and amino groups;
R₃ is selected from the group consisting of hydrogen, alkyl, alkenyl, alkynyl, aryl, heteroalkyl, heteroalkenyl, heteroalkynyl, aryl, acyl, heteroacyl, heteroaryl, aryl, alkyl, heteroarylalkyl, hydroxyl, alkoxy, halo, carbonyl, sulfinyl, sulfonyl, and amino groups;
R₄, R₅, R₆, R₇, R₈, R₉, R₁₀, R₁₁, R₁₂, and R₁₃ are each independently selected from the group consisting of hydrogen, halogen, alkyl, alkenyl, alkynyl, heteroalkyl, heteroalkenyl, heteroalkynyl, aryl, acyl, heteroacyl, heteroaryl, aryl, alkyl, heteroarylalkyl, hydroxyl, alkoxy, carbonyl, sulfinyl, sulfonyl groups;
n is 0 1, 2, 3, 4, or 5; and
Y is —CR₁₂═CR₁₃—, sulfur, or oxygen.

63. A catalytic hybrid comprising a polynucleotide and a nucleic acid analog that together form the hybrid, wherein the hybrid catalyses a chemical reaction of a dye upon exposure to a light stimulus.

64. The catalytic hybrid of claim 41, wherein the dye is a compound of formula (I), or a salt thereof: 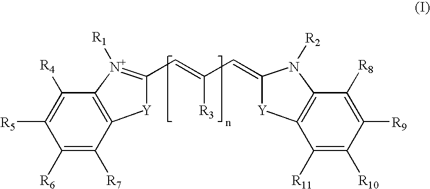
wherein, independently at each occurrence:
R₁ and R₂ are each independently selected from hydrogen, alkyl, alkenyl, alkynyl, heteroalkyl, heteroalkenyl, heteroalkynyl, aryl, acyl, heteroacyl, heteroaryl, hydroxyl, alkoxy, carbonyl, sulfinyl, sulfonyl, and amino groups;
R₃ is selected from the group consisting of hydrogen, alkyl, alkenyl, alkynyl, aryl, heteroalkyl, heteroalkenyl, heteroalkynyl, aryl, acyl, heteroacyl, heteroaryl, aryl, alkyl, heteroarylalkyl, hydroxyl, alkoxy, halo, carbonyl, sulfinyl, sulfonyl, and amino groups;
R₄, R₅, R₆, R₇, R₈, R₉, R₁₀, R₁₁, R₁₂, and R₁₃ are each independently selected from the group consisting of hydrogen, halogen, alkyl, alkenyl, alkynyl, heteroalkyl, heteroalkenyl, heteroalkynyl, aryl, acyl, heteroacyl, heteroaryl, aryl, alkyl, heteroarylalkyl, hydroxyl, alkoxy, carbonyl, sulfinyl, sulfonyl groups;
n is 0 1, 2, 3, 4, or 5; and
Y is —CR₁₂═CR₁₃—, sulfur, or oxygen.

65. The catalytic hybrid of claim 64, wherein the dye is not selected from the following list: 3,3′-Diethylthiacyanine; 3-Ethyl-9-methyl-3′-(3-sulfatobutyl)thiacarbocyanine; 3,3′-Dimethyloxacarbocyanine; 3-Carboxymethyl-3′,9-diethyl-5,5′-dimethylthiacarbocyanine; 3,3′-Diethylthiadicarbocyanine; 3,3′-Diethylthiatricarbocyanine; 3,3′-Diethyloxacarbocyanine; 3,3′-Diethyloxadicarbocyanine; 3,3′-Dipropylthiadicarbocyanine; 3,3′-Dipropyloxacarbocyanine; 3,3′-Dihexyloxacarbocyanine; 3,3′-Diethyl-2,2′-oxathiacarbocyanine; 1,1′-Diethyl-2,2′-cyanine; 1,1′-Diethyl-2,4′-cyanine; 1,1′-Diethyl-4,4′-carbocyanine; 1,1′-Diethyl-3,3,3′,3′-tetramethylindocarbocyanine; 1,1′-Dipropyl-3,3,3′,3′-tetramethylindocarbocyanine; [5-[2-(3-Ethyl-3H-benzothiazol-2-ylidene)-ethylidene]-4-oxo-2-thioxo-thiazolidin-3-yl]-acetic acid; 1-Butyl-2-[3-(1-butyl-1H-benzo[cd]indol-2-ylidene)-propenyl]-benzo[cd]indolium; 5,6-Dichloro-2-[3-(5,6-dichloro-1,3-diethyl-1,3-dihydro-benzimidazol-2-ylidene)-propenyl]-1,3-diethyl-3H-benzimidazolium; 1,3,3-Trimethyl-2-(2-[2-phenylsulfanyl-3-[2-(1,3,3-trimethyl-1,3-dihydro-indol-2-ylidene)-ethylidene]-cyclohex-1-enyl]-vinyl)-3H-indolium; 4,5,4′,5′-Dibenzo-3,3′-diethyl-9-methyl-thiacarbocyanine; and Thiazole orange.

66. The method of claim 1, wherein the quantity of the target polynucleotide is determined by comparing the observed optical is compared to a reference.

67. The method of claim 34, wherein the quantity of the target polynucleotide is determined by comparing the observed optical is compared to a reference.

68. The method of claim 62, wherein the quantity of the target polynucleotide is determined by comparing the observed optical is compared to a reference.
